(19) United States
(12) Patent Application Publication
(10) Pub. No.: US 2005/0118719 A1
(43) Pub. Date: Jun. 2, 2005

(54) Nucleic acid delivery and expression

(76) Inventors: Michael G. Schmidt, Charleston, SC (US); David A. Schofield, Mt. Pleasant, SC (US); Caroline Westwater, Mt. Pleasant, SC (US); Joseph W. Dolan, Mt. Pleasant, SC (US); Brian D. Hoel, James Island, SC (US); Philip A. Werner, Island of Palms, SC (US); James S. Norris, Mt. Pleasant, SC (US); Laura M. Kasman, Mt. Pleasant, SC (US)

Correspondence Address: FISH & RICHARDSON PC, 225 FRANKLIN ST, BOSTON, MA, 02110, US

(21) Appl. No.: 10/494,880

(22) PCT Filed: Nov. 7, 2002
(86) PCT No.: PCT/US02/35891

Related U.S. Application Data
(60) Provisional application No. 60/347,839, filed on Nov. 7, 2001.
(60) Provisional application No. 60/410,398, filed on Sep. 13, 2002.

(51) Int. Cl. C12N 15/74; C07H 21/04; C12N 1/21

(52) U.S. Cl. 435/471; 435/252.3; 435/320.1; 536/23.7

(57) ABSTRACT

The invention provides methods and materials involved in delivering nucleic acid to cells and regulating expression of nucleic acid in cells.

### BACKGROUND

1. Technical Field

The invention relates to methods and materials involved in nucleic acid delivery and nucleic acid expression. For example, the invention relates to methods and materials involved in bacteriophage mediated transformation of bacteria. In addition, the invention relates to regulated promoters such as highly stringent and dually regulated promoter systems functional in bacteria (e.g., Shigella flexneri).

2. Background Information

Nucleic acid vectors such as phagemids are useful constructs for transforming prokaryotic and eukaryotic cells. Phagemids can be modified to contain one or more nucleic acid sequences of interest under the control of suitable regulatory sequences. Currently, few useful vectors exist that are capable of (1) transforming a wide range of host cells and (2) providing a means for regulating the expression of desired nucleic acid in a wide range of host cells.

### SUMMARY

The invention provides methods and materials involved in nucleic acid delivery and nucleic acid expression. For example, the invention provides methods and materials for (1) transforming a wide range of host cells and (2) regulating the expression of one or more desired nucleic acid sequences in a wide range of host cells. The invention also relates to regulated promoters such as highly stringent and dually regulated promoter systems functional in bacteria (e.g., Shigella flexneri). In addition, the invention provides isolated nucleic acid, cells, phage, methods for inducing nucleic acid expression, and methods for repressing nucleic acid expression.

The nucleic acids and phage provided herein can be used to transform a wide range of host cells such as Gram-negative and Gram-positive bacteria. In addition, the provided nucleic acids can be used to regulate expression of one or more desired nucleic acid sequences in a wide range of host cells. The host cells provided herein can be used to produce various types of phage. For example, the provided host cells can be used to produce phage containing a transfer plasmid and not wild-type P1 genomic nucleic acid. Such phage can be used to deliver the transfer plasmid to a cell without allowing the cell to produce progeny phage.

In general, the invention features an isolated nucleic acid containing a C1-regulated promoter sequence operably linked to a nucleic acid sequence, and a promoter sequence operably linked to a second nucleic acid sequence, where the C1-regulated promoter sequence and the nucleic acid sequence are heterologous, and where the promoter sequence and the second nucleic acid sequence are heterologous. A cell containing the isolated nucleic acid can express at least about 10 times less of the nucleic acid sequence when the cell expresses a C1 polypeptide than when the cell does not express the C1 polypeptide. The cell can be a gram-negative bacterial cell (e.g., a cell that is a member of a family selected from the group consisting of Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, and Chromobacterium). The cell can be a gram-positive bacterial cell (e.g., a cell that is a member of a family or genus selected from the group consisting of Bacillaceae, Sporolactobacillus, Sporocarcina, Filibacter, Caryophanum, Peptococcus, Peptostreptococcus, Ruminococcus, Sarcina, Coprococcus, Mycobacteriaceae, Actinomyces, Bifidobacerium, Eubacterium, Propionibacerium, Staphylococci, Streptococci, Lactococcus, Lactobacillus, Corynebacterium, Erysipelothrix, and Listeria). A cell containing the isolated nucleic acid can express at least about 100 times less of the nucleic acid sequence when the cell expresses a C1 polypeptide than when the cell does not express the C1 polypeptide. A cell containing the isolated nucleic acid can express at least about 1000 times less of the nucleic acid sequence when the cell expresses a C1 polypeptide than when the cell does not express the C1 polypeptide. The C1-regulated promoter sequence can contain a sequence at least about 60 percent identical to the sequence set forth in SEQ ID NO:2, SEQ ID NO:3, SEQ ID NO:4, SEQ ID NO:5, SEQ ID NO:18, or SEQ ID NO:19. The C1-regulated promoter sequence can contain a sequence at least about 75 percent identical to the sequence set forth in SEQ ID NO:2, SEQ ID NO:3, SEQ ID NO:4, SEQ ID NO:5, SEQ ID NO:18, or SEQ ID NO:19. The C1-regulated promoter sequence can contain a sequence at least about 85 percent identical to the sequence set forth in SEQ ID NO:2, SEQ ID NO:3, SEQ ID NO:4, SEQ ID NO:5, SEQ ID NO:18, or SEQ ID NO:19. The C1-regulated promoter sequence can contain a sequence at least about 95 percent identical to the sequence set forth in SEQ ID NO:2, SEQ ID NO:3, SEQ ID NO:4, SEQ ID NO:5, SEQ ID NO:18, or SEQ ID NO:19. The nucleic acid sequence can encode a polypeptide (e.g., a bacterial polypeptide). Expression of the polypeptide in a bacterial cell can kill the bacterial cell. The polypeptide can be a Doc polypeptide. The nucleic acid sequence can encode an antisense nucleic acid or a ribozyme. The promoter sequence can be an inducible promoter sequence. The inducible promoter sequence can be an AraBAD promoter sequence, a T7 promoter sequence, a LacR10 promoter sequence, a TetR/O promoter sequence, or an AraC/IL-12 promoter sequence. The inducible promoter sequence can be a LacI-regulated promoter sequence. The LacI-regulated promoter sequence can contain a sequence at least about 60 percent identical to the E. coli LacI promoter. The LacI-regulated promoter sequence can contain a sequence at least about 75 percent identical to the E. coli LacI promoter. The LacI-regulated promoter sequence can contain a sequence at least about 85 percent identical to the E. coli LacI promoter. The LacI-regulated promoter sequence can contain a sequence at least about 95 percent identical to the E. coli LacI promoter. The second nucleic acid sequence can encode a polypeptide. The polypeptide can be a C1 polypeptide (e.g., a temperature sensitive C1 polypeptide). Binding of the temperature sensitive C1 polypeptide to the C1-regulated promoter sequence can be inhibited when the temperature is greater than 37° C. as compared to the binding that occurs at 31° C. Binding of the temperature sensitive C1 polypeptide to the C1-regulated promoter sequence can be inhibited when the temperature is greater than 40° C. as compared to the binding that occurs at 31° C. The promoter sequence can be a LacI-regulated promoter sequence. A cell containing the isolated nucleic acid can express at least about 10 times more of the nucleic acid sequence when the cell is exposed to 42° C. and 0 mM IPTG as compared to when the cell is exposed to 31° C. and 10 mM IPTG. The cell can be a gram-negative bacterial cell or a gram-positive bacterial cell). A cell containing the isolated nucleic acid can express at least about 100 times more of the nucleic acid sequence when the cell is exposed to 42° C. and 0 mM IPTG as compared to when the cell is exposed to 31° C. and 10 mM IPTG. A cell containing the isolated nucleic acid can express at least about 1000 times more of the nucleic acid sequence when the cell is exposed to 42° C. and 0 mM IPTG as compared to when the cell is exposed to 31° C. and 10 mM IPTG. The isolated nucleic acid can contain a sequence encoding a LacI polypeptide (e.g., a temperature sensitive LacI polypeptide). Binding of the temperature sensitive LacI polypeptide to the LacI-regulated promoter sequence can be inhibited when the temperature is greater than 37° C. as compared to the binding that occurs at 31° C. Binding of the temperature sensitive LacI polypeptide to the LacI-regulated promoter sequence can be inhibited when the temperature is greater than 40° C. as compared to the binding that occurs at 31° C. The nucleic acid sequence can encode a second polypeptide. A cell containing the isolated nucleic acid can express at least about 10 times more of the second polypeptide when the cell is exposed to 42° C. as compared to when the cell is exposed to 31° C. A cell containing the isolated nucleic acid can express at least about 100 times more of the second polypeptide when the cell is exposed to 42° C. as compared to when the cell is exposed to 31° C. A cell containing the isolated nucleic acid can express at least about 1000 times more of the second polypeptide when the cell is exposed to 42° C. as compared to when the cell is exposed to 31° C. The isolated nucleic acid can contain a sequence encoding a Bof modulator polypeptide. The Bof modulator polypeptide can contain an amino acid sequence at least about 60 percent identical to the sequence set forth in SEQ ID NO:7. The isolated nucleic acid can contain a sequence encoding a Coi polypeptide. The Coi polypeptide can contain an amino acid sequence at least about 60 percent identical to the sequence set forth in SEQ ID NO:8. The isolated nucleic acid can contain a pac site. The pac site can contain a nucleic acid sequence at least about 60 percent identical to the sequence set forth in SEQ ID NO:9, SEQ ID NO:10, or SEQ ID NO:11. The isolated nucleic acid can contain a transcription terminator sequence. The transcription terminator sequence can contain a nucleic acid sequence at least about 60 percent identical to the sequence set forth in SEQ ID NO:12 or SEQ ID NO:13.

In another aspect, the invention features an isolated cell containing nucleic acid, where the nucleic acid contains a C1-regulated promoter sequence operably linked to a nucleic acid sequence, and a promoter sequence operably linked to a second nucleic acid sequence, where the C1-regulated promoter sequence and the nucleic acid sequence are heterologous, and where the promoter sequence and the second nucleic acid sequence are heterologous. The cell can be a gram-negative bacterial cell (e.g., a cell that is a member of a family selected from the group consisting of Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, and Chromobacterium). The cell can be a gram-positive bacterial cell (e.g., a cell that is a member of a family or genus selected from the group consisting of Bacillaceae, Sporolactobacillus, Sporocarcina, Filibacter, Caryophanum, Peptococcus, Peptostreptococcus, Ruminococcus, Sarcina, Coprococcus, Mycobacteriaceae, Actinomyces, Bifidobacerium, Eubacterium, Propionibacerium, Staphylococci, Streptococci, Lactococcus, Lactobacillus, Corynebacterium, Erysipelothrix, and Listeria). The nucleic acid sequence can encode a polypeptide. The promoter sequence can be a LacI-regulated promoter sequence. The second nucleic acid sequence can encode a temperature sensitive C1 polypeptide. The C1-regulated promoter sequence, the nucleic acid sequence, the promoter sequence, and the second nucleic acid sequence can be located on the same nucleic acid molecule within the cell. The C1-regulated promoter sequence and the nucleic acid sequence can be located on chromosomal nucleic acid within the cell, and where the promoter sequence and the second nucleic acid sequence can be located on episomal nucleic acid within the cell. The nucleic acid can encode a temperature sensitive LacI polypeptide, a Bof modulator polypeptide, or a Coi polypeptide. The nucleic acid can contain a pac site or a transcription terminator sequence.

In aspect of the invention features an isolated P1 phage capsid containing nucleic acid, where the nucleic acid contains a pac site, a C1-regulated promoter sequence, and a nucleic acid sequence, where the C1-regulated promoter sequence is operably linked to the nucleic acid sequence, and where the C1-regulated promoter sequence and the nucleic acid sequence are heterologous. The nucleic acid sequence can encode a polypeptide. The nucleic acid can contain a promoter sequence operably linked to a second nucleic acid sequence. The promoter sequence can be a LacI-regulated promoter sequence. The second nucleic acid sequence can encode a temperature sensitive C1 polypeptide. The nucleic acid can encode a temperature sensitive LacI polypeptide, a Bof modulator polypeptide, or a Coi polypeptide. The nucleic acid can contain a transcription terminator sequence. Cells infected with the P1 phage capsid can produce progeny P1 phage capsids. The progeny P1 phage capsids can contain the nucleic acid. Cells infected with one or more of the progeny P1 phage capsids may not produce progeny P1 phage capsids.

In aspect of the invention features a method for inducing expression of a nucleic acid sequence within a cell, where the cell contains a nucleic acid containing (a) a C1-regulated promoter sequence operably linked to the nucleic acid sequence, and (b) a promoter sequence operably linked to a second nucleic acid sequence, where the second nucleic acid sequence encodes a temperature sensitive C1 polypeptide, the method including exposing the cell to a temperature greater than 36° C., thereby inducing expression of the nucleic acid sequence. The cell can be a gram-negative bacterial cell or a gram-positive bacterial cell. The temperature can be between about 37° C. and about 45° C.

In another embodiment, the invention features a method for repressing expression of a nucleic acid sequence within a cell, where the cell contains a nucleic acid containing: (a) a C1-regulated promoter sequence operably linked to the nucleic acid sequence, and (b) a promoter sequence operably linked to a second nucleic acid sequence, where the second nucleic acid sequence encodes a temperature sensitive C1 polypeptide, the method containing exposing the cell to a temperature less than 36° C., thereby repressing expression of the nucleic acid sequence. The temperature can be between about 25° C. and about 35° C.

In another embodiment, the invention features a method for repressing expression of a nucleic acid sequence within a cell, where the cell contains a nucleic acid containing: (a) a C1-regulated promoter sequence operably linked to the nucleic acid sequence, and (b) a LacI-regulated promoter sequence operably linked to a second nucleic acid sequence, where the second nucleic acid sequence encodes a temperature sensitive C1 polypeptide, the method containing exposing the cell to a temperature less than 36° C. and to IPTG, thereby repressing expression of the nucleic acid sequence. The temperature can be between about 25° C. and about 35° C.

In another embodiment, the invention features expression systems regulated by a bacteriophage P1 temperature sensitive C1 repressor polypeptide. The expression systems can function such that the induction/repression ratio is up to 1500-fold. The expression systems can exhibit extremely tight repression and can be modulated over a range of temperatures.

In another embodiment, the invention features a two component expression system that controls the amount of C1 polypeptide expressed at the mRNA level via a LacI-regulated promoter sequence. The expression system can result in an elevated level of induction (e.g., a greater than 10, 25, 50, 60, 70, 80, 90, 100, 125, 150, 175, 200, 500, or 1000-fold induction) in gene expression under inducing conditions in Gram-negative bacteria or in Gram-positive bacteria.

In another embodiment, the invention features expression constructs functional in a wide range of bacteria such as Shigella flexneri and Klebsiella pneumoniae. The expression constructs can contain C1 operator sites driving expression of a nucleic acid sequence (e.g., lacZ nucleic acid). The expression constructs can exhibit induction/repression ratios up to 240-fold in S. flexneri (e.g., at least about a 10, 25, 50, 60, 70, 80, 90, 100, 125, 150, 175, or 200-fold induction) and up to 50-fold in K. pneumoniae (e.g., at least about a 10, 20, 30, or 40-fold induction). The expression construct can exhibit low basal expression, can be modulated by temperature, and can exhibit rapid induction. The expression construct can control gene expression in enteric Gram-negative bacteria.

In another embodiment, the invention features delivery systems for transforming bacteria such as clinically important bacteria. The delivery systems can use the broad host range transducing bacteriophage P1.

In another embodiment, the invention features phagemids. The phagemids can contain a P1 pac initiation site to package the vector, a P1 lytic replicon to generate concatemeric DNA, a broad host range origin of replication, and an antibiotic-resistance determinant to select bacterial clones containing the recircularized phagemid. The phagemid DNA can be successfully introduced into cells by infection and stably maintained. The cells can be a member of a species from any family including Enterobacteriaceae (e.g., an Escherichia coli, Shigella flexneri, Shigella dysenteriae, Klebsiella pneumoniae, or Citrobacter freundii cell) and Pseudomonadaceae (e.g., an Pseudomonas aeruginosa cell). The cells can be cells from a laboratory strain or a strain isolated from a patient.

In another embodiment, the invention features methods for delivering nucleic acid for use in antimicrobial therapies and DNA vaccine development.

In another embodiment, the invention features recombinant nucleic acid vectors for regulated expression of genes. The gene can encode a polypeptide or a regulatory nucleic acid such as a catalytic nucleic acid (e.g., a ribozyme or DNAzyme) or antisense molecule. The vectors can contain a C1-regulated promoter sequence (e.g., an Op72 sequence), a sequence that encodes a temperature sensitive C1 repressor polypeptide, and a sequence that encodes a Bof modulator polypeptide. The vectors can containing a nucleic acid sequence encoding a C1 inactivator polypeptide (e.g., a Coi polypeptide). The vectors can contain a nucleic acid sequence encoding a LacI repressor polypeptide. The vectors can contain one or more transcriptional terminator sequences (e.g., a TL₁₇, rrnBT1, rrnBT2, or rrnBT1T2). The vectors can contain nucleic acid from pBBR122.

In another embodiment, the invention features transformation systems for transforming bacteria (e.g., Gram-positive bacteria or Gram-negative bacteria) containing modified bacteriophage having a phagemid. The phagemid can contain a pac initiation site, a lytic replicon, an origin of replication, and an antibiotic resistance determinant. The lytic replicon and the pac initiation site can be isolated from P1Cm clts100. The bacteriophage can be P1, P1kc, or P1Cm clts100. The phagemid can be P1pSK, P1pBBR122, P1p BBR122-T, or P1p BBR122-bla.

In another embodiment, the invention features phagemid vectors for delivering DNA to a wide range of bacterial species. The phagemid can contain a pac initiation site, a lytic replicon, an origin of replication, and an antibiotic resistance determinant. The lytic replicon and the pac initiation site can be isolated from P1Cm clts100. The phagemid can be P1pSK, P1p BBR122, P1p BBR122-T, or P1p BBR122-bla.

In another embodiment, the invention features transformation systems containing a modified bacteriophage having a phagemid. The phagemid can contain a bacteriophage initiation site, a lytic replicon to generate concatemeric DNA, an origin of replication, and an antibiotic resistance determinant.

In another embodiment, the invention features a highly stringent and dually regulated promoter system for Shigella flexneri. Dual regulation was provided by utilizing a promoter susceptible to control by the bacteriophage P1 temperature sensitive C1 repressor polypeptide that in turn was under the transcriptional control of a LacI polypeptide. The level of induction/repression ratios observed was up to 3700-fold in S. flexneri. The general utility of this promoter system was evaluated by demonstrating that the bacteriophage P1 post-segregational killer polypeptide Doc mediates a bactericidal effect in S. flexneri. This represents the first report of Doc-mediated killing in this Gram-negative species.

In another embodiment, the invention features a highly stringent and dually regulated promoter system for regulating the expression of one or more nucleic acids of interest (e.g., a nucleic acid that encodes a polypeptide of interest) in bacteria transformed with a construct containing the promoter system, wherein the one or more nucleic acids of interest encode(s) a bacterial toxin, a toxin derived from bacteriophage, a bactericidal polypeptide, a polypeptide derived from an animal, a polypeptide derived from a plant, a polypeptide derived from a bacterial species, or a polypeptide derived from bacteriophage; and wherein the transformed bacteria is selected from the group consisting of Gram-negative bacteria (e.g., Shigella flexneri or Escherichia coli) and Gram-positive bacteria.

In another embodiment, the invention features a vector containing a highly stringent and dually regulated promoter system for regulating the expression of one or more nucleic acids of interest (e.g., a nucleic acid that encodes a polypeptide of interest) in bacteria transformed with the vector, wherein the one or more nucleic acids of interest encode a bacterial toxin, a toxin derived from bacteriophage, a bactericidal polypeptide, a polypeptide derived from an animal, a polypeptide derived from a plant, a polypeptide derived from a bacterial species, or a polypeptide derived from bacteriophage; and wherein the transformed bacteria is selected from the group consisting of Gram-negative bacteria (e.g., Shigella flexneri or Escherichia coli) and Gram-positive bacteria.

In another embodiment, the invention features a host cell containing a vector. The vector contains a highly stringent and dually regulated promoter system for regulating the expression of one or more nucleic acids of interest (e.g., a nucleic acid that encodes a polypeptide of interest) in bacteria transformed with the vector, wherein the one or more nucleic acids of interest encode a bacterial toxin, a toxin derived from bacteriophage, a bactericidal polypeptide, a polypeptide derived from an animal, a polypeptide derived from a plant, a polypeptide derived from a bacterial species, or a polypeptide derived from bacteriophage; and wherein the transformed bacteria is selected from the group consisting of Gram-negative bacteria (e.g., Shigella flexneri or Escherichia coli) and Gram-positive bacteria.

In another embodiment, the invention features a method of transforming a host cell. The method includes introducing a vector into a host cell. The vector contains a highly stringent and dually regulated promoter system for regulating the expression of one or more nucleic acids of interest (e.g., a nucleic acid that encodes a polypeptide of interest) in bacteria transformed with the vector, wherein the one or more nucleic acids of interest encode a bacterial toxin, a toxin derived from bacteriophage, a bactericidal polypeptide, a polypeptide derived from an animal, a polypeptide derived from a plant, a polypeptide derived from a bacterial species, or a polypeptide derived from bacteriophage; and wherein the transformed bacteria is selected from the group consisting of Gram-negative bacteria (e.g., Shigella flexneri or Escherichia coli) and Gram-positive bacteria.

In another embodiment, the invention features a method of killing bacteria. The method includes expressing a polypeptide under the control of a regulated promoter system provided herein.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagram of a nucleic acid molecule containing a C1-regulated promoter driving expression of a sequence of interest (e.g., lacZ). The lacZ reporter sequence is expressed from a C1-regulated promoter (designated Pₐ) such as Op72 or AP, while the sequence encoding the thermally unstable C1 repressor polypeptide (designated c1) is expressed from a separate promoter (designated P_{b}) such as a LacI-regulated promoter. At the permissive temperature (31° C.), temperature sensitive C1 polypeptide binds to the Pₐ promoter and prevents transcription of the lacZ gene. At the non-permissive temperature (42° C.), the thermal instability of the temperature sensitive C1 polypeptide results in lacZ expression. In E. coli, when P_{b} is a LacI-regulated promoter, the chromosomal encoded LacI polypeptide binds to the P_{b} promoter thereby reducing C1 polypeptide expression. The addition of IPTG, which binds to LacI, can induce C1 polypeptide expression.

FIG. 2 is a diagram of a nucleic acid molecule containing a C1-regulated promoter driving expression of a sequence of interest (e.g., lacZ). Nucleic acid encoding a C1 inactivator polypeptide, Coi, is transcriptionally coupled to the lacZ reporter gene such that both are expressed from a C1-regulated promoter (designated Pₐ) such as Op72 or AP. The Coi polypeptide exerts its C1-inactivating function by forming a complex with the C1 repressor polypeptide, thereby inhibiting the binding of the C1 polypeptide to the operator sites of Pₐ. At the permissive temperature (31° C.), C1 polypeptide is stable, and an equilibrium exists between the levels of C1 polypeptide bound operators and C1.Coi polypeptide complex. Binding of C1 polypeptide to the operator site located in promoter Pₐ prevents expression of lacZ and coi. At the non-permissive temperature (42° C.), C1 polypeptide instability results in LacZ and Coi expression.

FIG. 3 is a diagram of a nucleic acid molecule containing a C1-regulated promoter driving expression of a sequence of interest (e.g., lacZ). Nucleic acid encoding a LacI polypeptide is transcriptionally coupled to the lacZ reporter gene, while nucleic acid encoding the C1 polypeptide is expressed from P_{b}, which in this case is a LacI-regulated promoter. At the permissive temperature and in the absence of IPTG, an equilibrium exists between LacI switching off (I) C1 polypeptide expression, and (2) C1 polypeptide repression of lacZ and lacI expression. Exposure to IPTG induces C1 polypeptide expression by titrating out the LacI repressor polypeptide. At the non-permissive temperature and in the absence of IPTG, C1 polypeptide instability results in LacZ and LacI expression which in turn switches off the promoter driving C1 polypeptide expression.

FIG. 4 is a diagram of transfer plasmids P1pBBR122, P1pSK, and P1pBBR122-T. The nucleic acid sequences encoding the mobilization (mob), replication (rep), and kanamycin resistance marker (kan) are derived from the broad host range cloning vector pBBR122. The nucleic acid sequences encoding the ampicillin resistance marker (bla) is derived from pBluescript IISK+. Sequences originating from the P1 bacteriophage include the packaging site (pac) and lytic replicon.

FIG. 5 is a listing of the nucleic acid sequence for four promoters. The Op72 and artificial promoter (AP) promoters are C1-regulated promoters. The Op72 promoter consists of two partially overlapping C1 operators (top and bottom strand as indicated by the underlined sequences). The top C1 operator site matches the 17 bp consensus, while the bottom operator deviates from the consensus by two nucleotides (circled bases). The proposed −10 and −35 promoter elements are shown in bold. The AP promoter contains a consensus C1-operator site flanked by consensus −10 and −35 hexamers. Pro3 and Pro4 drive can be used to drive C1 polypeptide expression. Pro3 contains of consensus hexamers, while Pro4 contains two mismatches from consensus.

FIG. 6 is a diagram of the Op721acZC1pBBR122 vector with various features identified. The lacZ reporter gene vectors were constructed in the broad host range Gram-negative plasmid pBBR122 (MoBiTec). The vector was modified to contain two antibiotic resistant markers to facilitate selection. The expression cassette is flanked by terminators at the 5′ and 3′ ends.

FIG. 7 is a graph plotting the amount of β-Gal activity (Miller Units) exhibited in S. flexneri (closed circles) and K. pneumoniae (open circles) carrying an Op72C1* reporter construct for the indicated temperatures. * indicates that the Pro4 promoter is driving c1.

FIG. 8 is a graph plotting the amount of β-Gal activity (Miller Units) exhibited in S. flexneri (closed circles) and K. pneumoniae (open circles) carrying an Op72C1* reporter construct for the indicated times at 42° C. * indicates that the Pro4 promoter is driving c1.

FIG. 9 is a graph plotting the amount of β-Gal activity (Miller Units) exhibited in E. coli DH5a (closed circles), TB1 (open triangles), and ER1793 (open circles) carrying an Op721acZ construct and incubated 2 hours at the indicated temperatures. Values reported (±standard deviation) are averages of duplicate cultures assayed in triplicate.

FIG. 10 depicts results demonstrating transduction of P1pBBR122-T into E. coli isolates. (a) The antibiotic-resistant phenotype conferred by phage infection and delivery of the phagemid is shown in the panels. The ability of bacteriophage P1 to infect and transduce laboratory and clinical isolates of E. coli was determined by infecting cells at an moi of 10⁻², 10⁻³, or 10⁻⁴. Ten-fold serial dilutions of cultures infected with phage were spotted vertically on media containing 50 μg kanamycin mL⁻¹. (b) Restriction digest analysis of E. coli transductants. Plasmid DNA isolated from the parent strain and two representative kanamycin resistant colonies from each infection were digested with HindIII and analyzed by agarose gel electrophoresis. Lane M, 1 kb DNA ladder; lanes 1-2, C600; lanes 3-4, JM101; lanes 5-6, DH5a; lane 7, control DNA from parent strain; lanes 8-9, JM101 P1 lysogen; lanes 10-11, JM109; lanes 12-13, EC-1 and lanes 14-15, EC-2. Predicted DNA fragments generated following HindIII digestion are 3332 and 3951 bp. Positions of molecular size standards are indicated on the left.

FIG. 11 depicts results demonstrating transduction of P1pBBR122-T carrying the b/a gene into P. aeruginosa. (a) The ability of bacteriophage P1 to infect and transduce laboratory and clinical isolates of P. aeruginosa was determined by infecting cells at an moi of 10⁻¹, 10⁻², or 10⁻³. Ten-fold serial dilutions of cultures infected with phage were spotted vertically on media containing carbenicillin at 500 μg mL⁻¹. Successful delivery and replication of the phagemid can be visualized by acquisition of the antibiotic marker bla. (b) Restriction digest analysis of P. aeruginosa transductants. Plasmid DNA isolated from the parent strain and two representative carbenicillin resistant colonies from each infection were digested with BamHI and analyzed by agarose gel electrophoresis. Lane M, 1 kb DNA ladder; lanes 1-2, PAO1; lane 3, control DNA from parent strain and lanes 4-5, PA-1. Predicted DNA fragments generated following BamHI digestion are 7920 and 42 bp. Positions of molecular size markers are indicated on the left.

FIG. 12 depicts results demonstrating transduction of P1pBBR122-T into K. pneumoniae, C. freundii, S. flexneri, and S. dysenteriae. (a) Bacterial species were infected by P1 at an moi of 10⁻², 10⁻³, and 10⁻⁴ and ten-fold serial dilutions of cultures infected with phage were spotted vertically on media. Presumptive transductants harboring the phagemid P1pBBR122-T were selected by virtue of their resistance to kanamycin at 50 μg mL⁻¹. (b) Restriction digest analysis of K. pneumoniae and C. freundii transductants. Plasmid DNA isolated from the parent strain and two representative kanamycin resistant colonies from each infection were digested with HindIII and analyzed by agarose gel electrophoresis. Lane M, 1 kb DNA ladder; lane 1, control DNA from parent strain; lanes 2-3, DNA isolated from kanamycin resistant transductants. Predicted DNA fragments generated following HindIII digestion are 3332 and 3951 bp. Positions of molecular size standards are indicated on the right. (c) Restriction digest analysis of S. flexneri and S. dysenteriae transductants. Control DNA or plasmid DNA isolated from kanamycin resistant colonies were digested with HindIII and analyzed by agarose gel electrophoresis. Lane M, 1 kb ladder; lane 1, control DNA isolated from parent strain; lane 2, S. flexneri and S. dysenteriae strains harboring an endogenous plasmid; lanes 3-4, transductants. Predicted DNA fragments generated following HindIII digestion are 3332 and 3951 bp. Positions of molecular size markers are indicated on the right.

FIG. 13 is a diagram of the stages of a Lethal Agent Delivery System, LADS™, which utilizes a bacteriophage based in vivo packaging system to create a targeted phage head, which acts as a molecule syringe, capable of delivering naturally occurring molecules with bacteriocidal activity to drug resistant bacteria.

FIG. 14 is a diagram of Op721acZpAM401 and lacIpBBR122. For Op72lacZpAM401, the lacZ gene was placed under the control of the Op72 promoter. To control gene expression, the temperature-sensitive C1 polyp peptide (sequence designated c1) was placed under the transcriptional control of a LacI-regulated promoter. Where indicated, lacZ was excised and doc was cloned into the respective sites. The expression cassette is flanked by terminators at the 5′ (labeled rrnBT1T2) and 3′ (labeled TL₁₇) ends. For lacIpBBR122, the lacI gene was cloned into the chloramphenicol resistance gene of the broad-host-range plasmid pBBR122. Transcriptional expression of lacI therefore relied on either cryptic promoters in the plasmid and/or the promoter driving the chloramphenicol resistance gene.

FIG. 15 depicts a Northern blot analysis of lacZ expression in E. coli and S. flexneri. Overnight cultures were diluted 1:100 and grown to an OD₆₀₀ of about 0.15 in LB containing 1 mM IPTG (S. flexneri, lanes 1-4) or 60 μM IPTG (E. coli, lanes 5-8) at 31° C. Cells were collected at 2, 500×g for 10 minutes at room temperature and resuspended in fresh LB. Cultures were then divided equally and incubated at 31° C. with additional IPTG (repressed, lanes 1, 3, 5, and 7) or at 42° C. without IPTG (induced, lanes 2, 4, 6, and 8) for 90 minutes. Control cultures (lanes 1, 2, 5, and 6) carried a promoterless lacZ construct, while the test cultures (lanes 3, 4, 7, and 8) carried the lacZ/lacI expression plasmids. RNA was prepared (Qiagen Rneasy), and Northern blot analysis was performed using a lacZ fragment random primed labeled with [α³²P]dCTP. The blot was reprobed with a ³⁵S-tailed oligonucleotide (5′-ACTTTATGAGGTCCGCTTGCTCTCGC, SEQ ID NO:1) complementary to both E. coli and S. flexneri 16s rRNA.

FIG. 16 contains two graphs. One graph plots the effect of Doc expression on the growth of S. flexneri. Overnight cultures, grown under repressed conditions (31° C., 1 mM IPTG), were diluted 1:100 and grown for 130 minutes under identical conditions. Cells were collected at 2, 500×g for 10 minutes at room temperature and resuspended in fresh LB. Cultures harboring the doc/lacI expression plasmids were then divided equally and incubated at 31° C. with additional IPTG (closed circles) or at 42° C. without IPTG (open circles). Control cultures harboring the lacZ/lacI plasmids were also grown under both repressed (closed squares) and induced conditions (open squares). The arrows denote time points at which samples were taken to determine viable counts. The other graphs the ability of S. flexneri to recover from Doc expression. Samples from cultures harboring the doc/lacI expression plasmids (open bars) were taken at 0 and 80 minutes induction (arrows on first graph) and plated in triplicate onto selective medium and grown under repressed conditions (31° C., 1 mM IPTG). As a control, the number of colony forming units were also measured for cultures harboring the lacZ/lacI plasmids (closed bars) incubated under the same conditions.

FIG. 17 is a listing of the indicated promoters. The conserved Gram-positive nucleotides based upon compilation analysis from Gram-positive promoters are shown in bold. The Ban promoter sequence (SEQ ID NO:2) is similar to the sequence of Op72. The synthetic promoters (P101, SEQ ID NO:3; P102, SEQ ID NO:4; and P103, SEQ ID NO:5) contain two partially overlapping C1 operators (top and bottom strand as indicated by the underlined sequences). P101 carries two C1 operator sites that match the 17 bp consensus, while P102 and P103 deviate from the consensus by one and five nucleotides, respectively (large font). P102 differs from P101 by a single nucleotide in the −10 hexamer (G to the consensus T). P103 differs from P102 by two nucleotide changes in the spacer region (AT to the consensus TG). P201 and P202, which were used to drive C1 polypeptide expression, differ in the nucleotide spacer sequence between the −35/−10 hexamers.

FIG. 18 is a diagram of the reporter plasmid and its relevant features. The lacZ reporter gene was placed under the transcriptional control of a C1-regulated promoter (either P101, P102, or P103; arrow denotes direction). To control gene expression and to aid binding of the repressor to its operator site, nucleic acid encoding the temperature sensitive C1 repressor polypeptide and the Bof modulator polypeptide were cloned 3′ of lacZ and placed under the transcriptional control of either P201 or P202. To stop read-through from cryptic promoters and to prevent runaway transcription, transcriptional terminators TL₁₇ were cloned 5′ and 3′ of the expression cassette. The reporter construct contains the p15A origin of replication, the origin of replication derived from pGB354, and the chloramphenicol (Cm) resistance markers from pACYC184 and pGB354.

FIG. 19 is a graph the levels of β-Gal activity from temperature sensitive C1-regulated promoters in S. aureus (closed circles), E. faecium (open circles), and E. faecalis (closed triangles) at the indicated temperatures. Overnight cultures carrying the reporter construct were diluted 1:100 and grown at 31° C. The culture was then divided equally and incubated for 75 minutes (S. aureus), 120 minutes (E. faecium), or 95 minutes (E. faecalis) at the designated temperatures prior to assaying for β-Gal activity (OD₆₀₀ at time of harvesting about 0.6). Values (±standard deviation) are averages of triplicate cultures assayed in triplicate. The reporter constructs used for each species is denoted in Table 12.

FIG. 20 is a graph plotting the time course of temperature induction of lacZ expression. Overnight cultures carrying the reporter constructs were diluted 1:100 and grown at 31° C. to early-log phase. Aliquots of the culture were then incubated at 42° C. for the indicated times in a staggered fashion so that the OD₆₀₀ at the time of harvesting for β-Gal assays was about 0.6. Values reported (±standard deviation) are averages of duplicate cultures assayed in triplicate. The reporter constructs used for each species is shown in Table 12.

FIG. 21 is a diagram outlining the generation of a P1 pac site knockout. The disruption cassette contains a nutritional or antibiotic marker flanked by sequences homologous to the P1 prophage. The linear fragment is protected from exonuclease attack by the incorporation of phosphorothioate groups. A double crossover event between the in vitro-altered sequence and the P1 prophage results in deletion of the pac site and acquisition of the selectable marker.

FIG. 22 is a diagram of a transfer plasmid. (A) The transfer plasmid containing the essential signals for packaging (a pac site and a lytic replicon under the control of the P1 P53 promoter), a selectable marker for detection (bla, ampicillin), and ColE1 origin for replication in E. coli. (B) The lytic replicon contains a C1-regulated promoter (e.g., the C1-regulated P53 promoter designated P53), the promoter P53 antisense, and genes kilA and repL. The kilA gene contains an in frame deletion that truncates the coding sequence such that only about half of the polypeptide is produced. P53 antisense can play a role in the stability of the P1 replicon.

FIG. 23 is a diagram depicting the delivery efficiency of the transfer plasmid by the P1 system to E. coli. The E. coli P1Cm c1ts100 lysogen carrying the transfer plasmid was induced by thermal induction to produce phage particles. Phage lysates were treated with DNase and RNase, and precipitated particles were resuspended in 50 mM Tris-Cl pH 7.5, 10 mM MgSO₄, 5 mM CaCl₂, 0.01% gelatin. E. coli C600 and E. coli P1 C600 target cells (105 CFU/mL, treated with 10 mM MgSO₄, 5 mM CaCl₂) were infected with each of the phage lysates. Following 30 minutes incubation at 30° C., infections were plated onto selection plates and antibiotic resistant colonies were scored. Values indicate number of antibiotic resistant colonies±standard error, n=6.

FIG. 24 depicts results from the identification of the P1 pac site knockout by PCR screening. The top panel shows the physical map of the P1 prophage and predicted P1 knockout following integration of the disruption cassette at the pac site. Arrows indicate location of the PCR primers used to verify the replacement of the P1 pac site with the S. cerevisiae TRP1 gene. The gels show the products of the PCRs using P1 specific primers (1, 3, 5, and 6) and disruption cassette specific primers (2 and 4) to detect either the wild-type P1 prophage or the P1 knockout. Primers 1 and 3 do not bind within the P1 sequences in the disruption cassette therefore PCR with primers 1+2 and 3+4 only detects a specific integration event which results in replacement of the pac site with the S. cerevisiae TRP1 gene.

FIG. 25 is a diagram of apacABC complementing plasmid. P1 pacABC are expressed from an early promoter Pr94. Two phage encoded polypeptides, C1 repressor and Bof modulator, are used to regulate expression from the Pr94 promoter.

FIG. 26 contains results from the recombination between the P1 pac mutant and pacABC complementing plasmid. P1 pac mutant lysogens harboring the transfer plasmid and pacABC complementing plasmid were grown at 32° C. and diluted 1:100 into fresh medium every 16 hours. DNA was extracted on day 1, 2, 3, 4, and 5, digested with HindIII, and probed with a ScTRP1 EcoRI-BamHI fragment under high stringency conditions.

FIG. 27 is a listing of the 162 bp pac site sufficient to promote pac cleavage and P1 packaging. The positions of the hexanucleotide elements within the Hex4 and Hex3 domains are shown by open boxes. The IHF binding site, consensus sequence 5′-AATCAANNANTTA (SEQ ID NO:6), is indicated underneath. Regulation of pac cleavage involves adenine methylation at 5′-GATC sites (within each open box). Silent mutations introduced into the pac site are indicated by lower case letters.

### DETAILED DESCRIPTION

The invention provides methods and materials involved in nucleic acid delivery and nucleic acid expression. For example, the invention provides methods and materials for (1) transforming a wide range of host cells and (2) regulating the expression of one or more desired nucleic acid sequences in a wide range of host cells. Such methods and materials include isolated nucleic acid, cells, phage, methods for inducing nucleic acid expression, and methods for repressing nucleic acid expression.

1. Nucleic Acid Molecules

The invention provides isolated nucleic acids that can be used to control expression of one or more nucleic acid sequences. The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a bacteriophage, retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a bacteriophage, retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

Typically, the isolated nucleic acids of the invention contain one or more C1-regulated promoter sequences. A C1-regulated promoter sequence is any nucleic acid sequence that directs transcription of another nucleic acid sequence in a manner regulated by either (1) the C1 polypeptide set forth at GenBank® accession number X16005 or (2) the temperature sensitive C1 polypeptide described by Heinrich et al. (temperature sensitive mutant P1c1.100; Heinrich et al., Nucleic Acids Res., 17(19):7681-92 (1989)). The amino acid sequence of the temperature sensitive C1 polypeptide described by Heinrich et al. is encoded by the nucleic acid sequence set forth at GenBank® accession number X16005 with the following two changes: a Gly to Cys change at the codon with nucleotide number 779 and a Leu to Pro change at the codon with nucleotide number 787.

While not being limited to any specific mode of action, a promoter sequence provides sequence-specific binding sites for nucleic acid binding polypeptides including, but not limited to, transcription factors, modulators, and repressors, and it is presumably the binding of a nucleic acid binding polypeptide to a promoter sequence that regulates the transcription of another nucleic acid sequence. The promoter and the nucleic acid sequence regulated by the promoter must be located on the same nucleic acid molecule for regulated expression to occur. The distance, however, between the promoter and the regulated sequence can be any distance, provided regulation occurs. For example, a promoter sequence, such as Op72, can be a few bases upstream of a sequence to be regulated. Alternatively, a promoter sequence can function like an enhancer in that it can be a few hundred kilobases upstream or downstream of a sequence to be regulated. In both cases, the promoter sequence and the regulated sequence are considered operably linked. The term “operably linked” as used herein with respect to a promoter sequence means that the functional relationship between the promoter sequence and the nucleic acid sequence to be regulated is intact such that transcription of the regulated nucleic acid sequence can occur. Further, promoter sequences can be in any orientation with respect to the nearby nucleic acid sequence. For example, a promoter sequence can be 5′-XXY-3′ or inverted to read 5′-YXX-3′. In addition, nucleic acid binding polypeptides can function in conjunction with other nucleic acid binding polypeptides such that the binding to a particular promoter sequence is influenced.

Common molecular biology techniques can be used to operably link a promoter sequence to a nucleic acid sequence to be regulated such that the promoter sequence drives transcription of the to be regulated nucleic acid sequence.

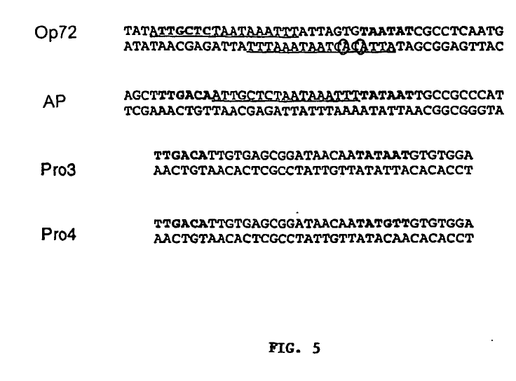

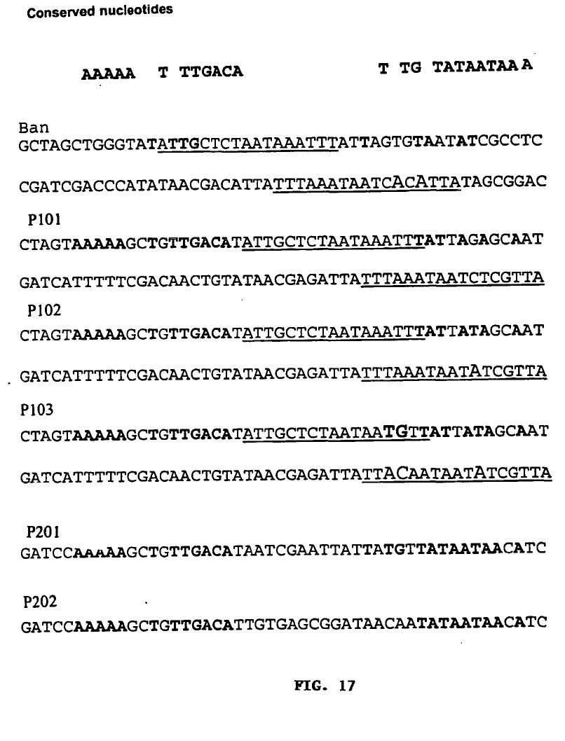

Any C1-regulated promoter sequence can be used such as Op72, AP, Ban, P101, P102, and P103 (FIGS. 5 and 17). In addition, C1-regulated promoter sequences can be designed as described herein. For example, a nucleic acid sequence can be designed to contain a sequence having a mutated C1 polypeptide binding site. Such sequences can be tested for promoter activity using standard assays involving a reporter sequence such as a lacZ.

A C1-regulated promoter sequence can contain a sequence at least about 60 percent (e.g., at least about 65, 70, 75, 80, 85, 90, 95, or 99 percent) identical to the sequence of Op72, AP, Ban, P101, P102, or P103 (FIGS. 5 and 17).

The percent identity between two nucleic acid sequences or two amino acid sequences is determined as follows. First, two nucleic acid sequences or amino acid sequences are compared using the BLAST 2 Sequences (B12seq) program from the stand-alone version of BLASTZ containing BLASTN version 2.0.14 and BLASTP version 2.0.14. This stand-alone version of BLASTZ can be obtained from the State University of New York—Old Westbury campus library as well as at Fish & Richardson P.C.'s web site (World Wide Web at fr.com/blast/) or the U.S. government's National Center for Biotechnology Information web site (World Wide Web at ncbi.nlm.nih.gov). Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q −1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences.

The percent identity is determined by dividing the number of matches by the length of the sequence set forth in an identified sequence (e.g., SEQ ID NO:1) followed by multiplying the resulting value by 100. For example, if a sequence is compared to a sequence set forth in a sequence identifier with a length of 1000 and the number of matches is 900, then the sequence has a percent identity of 90 (i.e., 900-1000*100=90) to the sequence set forth in that sequence identifier.

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

A C1-regulated promoter sequence can contain a sequence that is at least about 10 bases in length (e.g., at least about 12, 14, 15, 16, 17, 18, 19, 20, 25, 30, 40, 50, 60, or 100 bases in length) and hybridizes, under hybridization conditions, to the sense or antisense strand of a nucleic acid having the sequence of Op72, AP, Ban, P101, P102, or P 103 (FIGS. 5 and 17). The hybridization conditions can be moderately or highly stringent hybridization conditions.

For the purpose of this invention, moderately stringent hybridization conditions mean the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate.

Highly stringent hybridization conditions mean the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

Typically, the isolated nucleic acids of the invention contain one or more nucleic acid sequences operably liked to a C1-regulated promoter sequences. Such nucleic acid sequences can encode a polypeptide or a catalytic nucleic acid (e.g., rybozyme or DNAzyme). For example, any of the nucleic acids described in PCT publication number WO 00/61804, WO 99/67400, or WO 01/79524 can be used. Other examples include nucleic acids that encode bacterial toxins, toxins derived from bacteriophage, bactericidal polypeptides, polypeptides derived from an animal, polypeptides derived from a plant, polypeptides derived from a bacterial species, and polypeptides derived from bacteriophage.

The nucleic acid sequence operably liked to the C1-regulated promoter sequence can be heterologous with respect to that C1-regulated promoter sequence. The term “heterologous” as used herein with reference to two nucleic acid sequences within a single nucleic acid molecule in nature.

The isolated nucleic acids of the invention can contain other promoter sequences such as constitutive promoter or inducible promoters. Examples of other promoter sequences include, without limitation, AraBAD promoter sequences, T7 promoter sequences, LacR/O promoter sequences, TetR/O promoter sequences, and AraC/IL-12 promoter sequences (Backman and Ptashne, 1978. Cell 13(1):65-71; Ben-Samoun, K., G. Leblon, and O. Reyes. 1999. FEMS Microbiol Lett 174(1):125-30; Brunschwig, E., and A. Darzins. 1992. Gene 111(1):35-41; Guzman, L. M., D. Belin, M. J. Carson, and J. Beckwith. 1995. Journal of Bacteriology 177(14):4121-30; Lutz, R., and H. Bujard. 1997. Nucleic Acids Research 25(6):1203-10; Newman, J. R., and C. Fuqua. 1999. Gene 227(2): 197-203; Sukchawalit, R., P. Vattanaviboon, R. Sallabhan, and S. Mongkolsuk. 1999. FEMS Microbiology Letters 181(2):217-223; Tabor, S., and C. C. Richardson. 1985. Proc Natl Acad Sci USA 82(4):1074-8). In addition, LacI-regulated promoter sequences can be used such a those described herein. LacI-regulated promoter sequences can be regulated by LacI polypeptides or temperature sensitive LacI polypeptides such as those described by Andrews et al. (Gene, 182:101-9 (1996)).

The promoter sequences can be operably linked to any nucleic acid sequence such as those described above. In some embodiments, the isolated nucleic acids of the invention are constructed to contain (1) a C1-regulated promoter sequence operably linked to a nucleic acid sequence of interest and (2) a LacI-regulated promoter (or any other promoter) operably linked to a nucleic acid sequence encoding a C1 polypeptide (e.g., a temperature sensitive C1 polypeptide). Such isolated nucleic acids can be used to regulate the expression of the nucleic acid sequence of interest as described in the Examples. When a LacI-regulated promoter is used, a nucleic acid encoding a LacI polypeptide can be added to the nucleic acid molecule or the cell containing the nucleic acid molecule. The LacI polypeptide can be a temperature sensitive LacI polypeptide such as those described by Andrews et al. (Gene, 182:101-9 (1996)).

C1 polypeptides can have the following amino acid sequence: MINYVYGEQLYQEFVSFRDLFLKKAVARAQHVDAASDGRPVRPVVVLPFKETDSIQAEIDKWT LMARELEQYPDLNIPKTILYPVPNILRGVRKVTTYQTEAVNSVNMTAGRIIHLIDK DIIUQKSAGINEHSAKYIENLEATKELMKQYPEDEKFRMRVHGFSETMLRVHYISS SPNYNDGKSVSYHVLLCGVFICDETLRDGIIINGEFEKAKFSLYDSIEPIICDRWPQ AKIYRLADIENVKKQIAITREEKKVKSAASVTRSRKTKKGQPVNDNPESAQ (SEQ ID NO:6). In addition, a C1 polypeptide can contain an amino acid sequence at least about 60 percent (e.g., at least about 65, 70, 75, 80, 85, 90, 95, or 99 percent) identical to the sequence set forth in SEQ ID NO:6. Alternatively, a C1 polypeptide can be encoded by a nucleic acid sequence that is at least about 40 bases in length (e.g., at least about 50, 60, 75, 80, 100, 200, 300, or 500 bases in length) and hybridizes, under hybridization conditions, to the sense or antisense strand of a nucleic acid having the sequence set forth at GenBank® accession number X16005. The hybridization conditions can be moderately or highly stringent hybridization conditions.

The isolated nucleic acids of the invention can contain one or more nucleic acid sequences that encode Bof modulator polypeptides. Bof modulator polypeptides can have the following amino acid sequence: MKKRYYTVKHGTLRALQEFADKHNVEVRREGGSKALRMYRPDGKWRTVVDFKTNSVPQGVRDRAFEEW EQIIIDNALLLNAD (SEQ ID NO:7). In addition, a Bof modulator polypeptide can contain an amino acid sequence at least about 60 percent (e.g., at least about 65, 70, 75, 80, 85, 90, 95, or 99 percent) identical to the sequence set forth in SEQ ID NO:7. Alternatively, a Bof modulator polypeptide can be encoded by a nucleic acid sequence that is at least about 25 bases in length (e.g., at least about 50, 60, 75, 80, 100, 200, 300, or 500 bases in length) and hybridizes, under hybridization conditions, to the sense or antisense strand of a nucleic acid encoding the sequence set forth in SEQ ID NO:7. The hybridization conditions can be moderately or highly stringent hybridization conditions.

The isolated nucleic acids of the invention can contain one or more nucleic acid sequences that encode C1 inactivator polypeptide (e.g., a Coi polypeptide). Coi polypeptides can have the following amino acid sequence: MAFIPPTIDDVRHCSNALSVDPAETDAARAIAEHYSKISNQEYRITQDDLDDLTDTIEYLMATNQPDSQ (SEQ ID NO:8). In addition, a Coi polypeptide can contain an amino acid sequence at least about 60 percent (e.g., at least about 65, 70, 75, 80, 85, 90, 95, or 99 percent) identical to the sequence set forth in SEQ ID NO:8. Alternatively, a Coi polypeptide can be encoded by a nucleic acid sequence that is at least about 25 bases in length (e.g., at least about 50, 60, 75, 80, 100, 200, 300, or 500 bases in length) and hybridizes, under hybridization conditions, to the sense or antisense strand of a nucleic acid encoding the sequence set forth in SEQ ID NO:8. The hybridization conditions can be moderately or highly stringent hybridization conditions.

The isolated nucleic acids of the invention can contain one or more pac sites. Pac sites can have one of the following nucleic acid sequences:

(SEQ ID NO:9)

AGCATGATCATTGATCACTCTAATGATCAACATGCAGGTGATCACATTGC
GGCTGAAATAGCGGAAAAACAAAGAGTTAATGCCGTTGTCAGTGCCGCAG
TCGAGAATGCGAAGCGCCAAAATAAGCGCATAAATGATCGTTCAGATGAT
CATGACGTGATCACGCGCGCCCACCGGACCTTACGTGATCGCCTGGAACG
CGACACCCTGGATGATGATGGTGAACGCTTTGAATTC;

(SEQ ID NO:10)

CATGATCATTGATCACTCTAATGATCAACATGCAGGTGATCACATTGCGG
CTGAAATAGCGGAAAAACAAAGAGTTAATGCCGTTGTCAGTGCCGCAGTC
GAGAATGCGAAGCGCCAAAATAAGCGCATAAATGATGGTTCAGATGATCA
TGACGTGATCAC;

(SEQ ID NO:11)

CCACTAAAAAGCATGATCATTGATCACTCTAATGATCAACATGCAGGTGA
TCACATTGCGGCTGAAATAGCGGAAAAACAAAGAGTTAATGCCGTTGTCA
GTGCCGCAGTCGAGAATGCGAAGCGCCAAAATAAGCGCATAAATGATCGT
TCAGATGATCATGACGTGATCACCCGC.

In addition, a pac site can contain a nucleic acid sequence at least about 60 percent (e.g., at least about 65, 70, 75, 80, 85, 90, 95, or 99 percent) identical to the sequence set forth in SEQ ID NO:9, SEQ ID NO:10, or SEQ ID NO:11. Alternatively, a pac site can be a nucleic acid sequence that is at least about 10 bases in length (e.g., at least about 12, 15, 20, 25, 30, 35, 40, 50, 60, 75, 80, 100, 200, 300, or 500 bases in length) and hybridizes, under hybridization conditions, to the sense or antisense strand of a nucleic acid encoding the sequence set forth in SEQ ID NO:9, SEQ ID NO:10, or SEQ ID NO:11. The hybridization conditions can be moderately or highly stringent hybridization conditions.

The isolated nucleic acids of the invention can contain one or more transcription terminator sequences. Transcription terminator sequences can have one of the following nucleic acid sequences:

(SEQ ID NO:12)

CCTGGCGGATGAGAGAAGATTTTCAGCCTGATACAGATTAAATCAGAACG
CAGAAGCGGTCTGATAAAACAGAATTTGCCTGGCGGCAGTAGCGCGGTGG
TCCCACCTGACCCCATGCCGAACTCAGAAGTGAAAGGCCGTAGCGCCGAT
GGTAGTGTGGGGTCTGCCCATGCGAGAGTAGGGAACTGCCAGGCATCAAA
TAAAACGAAAGGCTCAGTCGAAAGACTGGGCGTTTCGTTTTATCTGTTGT
TTGTCGGTGAACGCTCTCCTGAGTAGGACAAATCCGCCGGGAGCGGATTT
GAACGTTGCGAAGCAACGGCGCGGAGGGTGGCGGGCAGGACGCCCGCCAT
AAACTGCCAGGCATCAAATTAAGCAGAAGGCCATCCTGACGGATGGCCTT
TTTGC;

(SEQ ID NO:13)

TAAAAAAACCCGCCCCGGCGGGTTTTTTTA;

In addition, a transcription terminator sequence can contain a nucleic acid sequence at least about 60 percent (e.g., at least about 65, 70, 75, 80, 85, 90, 95, or 99 percent) identical to the sequence set forth in SEQ ID NO:12 or SEQ ID NO:13. Alternatively, a transcription terminator sequence can be a nucleic acid sequence that is at least about 10 bases in length (e.g., at least about 12, 15, 20, 25, 30, 35, 40, 50, 60, 75, 80, 100, 200, 300, or 500 bases in length) and hybridizes, under hybridization conditions, to the sense or antisense strand of a nucleic acid encoding the sequence set forth in SEQ ID NO:12 or SEQ ID NO:13. The hybridization conditions can be moderately or highly stringent hybridization conditions.

The isolated nucleic acids described herein can be obtained using any method including, without limitation, common molecular cloning and chemical nucleic acid synthesis techniques. For example, PCR can be used to obtain an isolated nucleic acid containing a nucleic acid sequence sharing similarity to the sequences set forth in a sequence identifier. PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complimentary DNA strands.

The isolated nucleic acids described herein also can be obtained by mutagenesis. For example, an isolated nucleic acid containing a sequence encoding a C1 polypeptide can be mutated using common molecular cloning techniques (e.g., site-directed mutagenesis). Possible mutations include, without limitation, deletions, insertions, and substitutions, as well as combinations of deletions, insertions, and substitutions.

In addition, nucleic acid and amino acid databases (e.g., GenBank®) can be used to obtain an isolated nucleic acids described herein. For example, any nucleic acid sequence having some homology to a sequence set forth herein, or any amino acid sequence having some homology to a sequence set forth herein, can be used as a query to search GenBank®.

Further, nucleic acid hybridization techniques can be used to obtain an isolated nucleic acid described herein. Briefly, any nucleic acid having some homology to a sequence described herein can be used as a probe to identify a similar nucleic acid by hybridization under conditions of moderate to high stringency. Once identified, the nucleic acid then can be purified, sequenced, and analyzed.

Hybridization can be done by Southern or Northern analysis to identify a DNA or RNA sequence, respectively, that hybridizes to a probe. The probe can be labeled with a biotin, digoxygenin, an enzyme, or a radioisotope such as ³²P. The DNA or RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring harbor Laboratory, Plainview, N.Y. Typically, a probe is at least about 20 nucleotides in length. For example, a probe corresponding to a 20 nucleotide sequence set forth in a sequence identifier can be used to identify an identical or similar nucleic acid. In addition, probes longer or shorter than 20 nucleotides can be used.

The isolated nucleic acids of the invention can be vectors capable of transforming bacteria such as Gram-negative and Gram-positive bacteria. Examples in bacteria from the following families and genera: Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, Chromobacterium, Bacillaceae (e.g., species from the Bacillus genera such as B. anthracis, B. azotoformans, B. cereus, B. coagulans, B. israelensis, B. larvae, B. mycoides, B. polymyxa, B. pumilis, B. stearothormophillus, B. subtilis, B. thuringiensis, or B. validus), Sporolactobacillus, Sporocarcina, Filibacter, and Caryophanum, Peptococcus (e.g., P. niger), Peptostreptococcus (e.g, Ps. Anaerobius), Ruminococcus, Sarcina, Coprococcus, Mycobacteriaceae, Actinomyces, Bifidobacerium, Eubacterium, Propionibacerium, Staphylococci (e.g., coagulase positive Staphyloccus aureus, coagulase negative Staphylococcus aureus, Staphylococcus epidermidis), Streptococci (e.g., S. pyogenes from, for example, Lancefield group A, S. agalactiae including members of the Lancefield group B, members of Lancefield group D recently reclassified as the genus Enterococcus including members of the species faecalis and faceium, and members of the viridins group such as S. mutans and S. mitis), Lactococcus, Lactobacillus, Corynebacterium, Erysipelothrix, and Listeria. The vectors can be capable of directing replication or insertion into a host chromosome. In addition, the vectors can direct the expression of nucleic acid as described herein.

In one embodiment, the vector containing a nucleic acid sequence will include a prokaryotic replicon (e.g., a DNA sequence having the ability to direct autonomous replication and maintenance of the recombinant DNA molecule extra-chromosomally in a prokaryotic host cell, such as a bacterial host cell, transformed therewith). Such replicons are well known in the art. In addition, vectors that include a prokaryotic replicon can also include a gene whose expression confers a detectable marker such as a drug resistance. Typical bacterial drug resistance genes include, but are not limited to, those that confer resistance to ampicillin, kanamycin, or tetracycline.

Vectors that include a prokaryotic replicon can further include a prokaryotic or bacteriophage promoter (e.g., a C1-regulated promoter) capable of directing the expression of nucleic acid sequences in a bacterial host cell such as E. coli, or any other Gram-negative or Gram positive bacteria. Promoter sequences compatible with bacterial hosts are typically provided in plasmid and phagemid vectors containing convenient restriction sites for insertion of a DNA segment of the present invention. Typical of such vector plasmids are pBBR122 (Mobitec), pBluescript (Stratagene), pUC8, pUC9, pBR322, and pBR329 available from BioRad Laboratories, (Richmond, Calif.), pPL and pKK223 available from Pharmacia (Piscataway, N.J.).

Expression vectors compatible with eukaryotic cells, preferably those compatible with vertebrate cells such as kidney cells, can also be used to form recombinant DNA molecules that contain a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the recombinant DNA molecules of the invention may further include a selectable marker that is effective in a eukaryotic cell, preferably a drug resistance selection marker. A preferred drug resistance marker is the gene whose expression results in neomycin resistance, i.e., the neomycin phosphotransferase (neo) gene. (Southern et al., Journal of Molecular and Applied Genetics, Vol. 1, no. 4 (1982) pp. 327-341) Alternatively, the selectable marker can be present on a separate plasmid, and the two vectors are introduced by co-transfection of the host cell, and selected by culturing in the appropriate drug for the selectable marker.

2. Cells

The invention provides cells containing any of the nucleic acids described herein. Such cells can express a desired nucleic acid sequence in a regulated manner. Typically, the cells contain a nucleic acid having (1) a C1-regulated promoter sequence operably linked to one nucleic acid sequence and (2) a promoter sequence operably linked to another nucleic acid sequence. Each nucleic acid sequence can be heterologous with respect to the promoter sequence that controls its expression. The cells can contain one or more nucleic acid molecules. For example, a cell can contain one nucleic acid molecule having a C1-regulated promoter sequence operably linked to a nucleic acid sequence and another nucleic acid molecule having a promoter sequence operably linked to a nucleic acid sequence. It is noted the nucleic acid within a cell can contain any of the sequences described herein (e.g., nucleic acid encoding a Bof polypeptide, a C1 polypeptide, or a Coi polypeptide).

The cells can be either prokaryotic or eukaryotic. Eukaryotic cells include, but are not limited to, yeast, insect, mammalian cells, vertebrate cells such as those from a mouse, rat, monkey, or human cell line. Examples of eukaryotic cells that can be used include, without limitation, Chinese hamster ovary (CHO) cells available from the ATCC as CCL61, NIH Swiss mouse embryo cells (NIH3T3) available from the ATCC as CRL 1658, baby hamster kidney cells (BHK), COS and COS7 cells and like eukaryotic tissue culture cell lines.

Any prokaryotic cell can be used such as the following:

| | Gram-negative | Gram-negative |
|---|---|---|
| Gram-negative | | |
| Citrobacter freundii | Agrobacterium tumefaciens | Acetobacter xylinum |
| Escherichia coli | Alcaligenes faecalis | |
| | | Alcaligenes eutrophus |
| Klebsiella oxytoca | Enterobacter aerogenes | Bartonella bacilliformis |
| Klebsiella pnuemoniae | Enterobacter cloacae | Bordetella spp. |
| Pseudomonas aeruginosa | | Brucella spp. |
| | Enterobacter liquifaciens | |
| Shigella dysenteriae | Erwina amylovora | Burkholderia spp. |
| Shigella flexneri | Erwina carotovora | Caulobacter crescentus |
| | Flavobacterium spp. | |
| | | Flavobacterium heparium |
| | Klebsiella aerogenes | Paracoccus denitrificans |
| | Myxococcus xanthus | Pseudomonas fluorescens |
| | Proteus inconstans | Pseudomonas putida |
| | Proteus mirabilis | Rhizobium leguminosarum |
| | Proteus vulgaris | Rhizobium meliloti |
| Gram-positive | | |
| Enterococcus faecalis | Pseudomonas amyloderamosa | Rhodobacter sphaeroides |
| Staphylococcus aureus | Salmonella typhi | Salmonella typhimurium |
| | Salmonella typhimurium | Vibrio cholerae |
| | Serratia marcescens | Xanthomonas campestris |
| | Yersina pestis | |
| | Yersina pseudotuberculosis | |

3. Phage

The invention provides phage and phage capsids containing any of the nucleic acids described herein. Typically, the phage and phage capsids contain a nucleic acid having (1) a C1-regulated promoter sequence operably linked to a nucleic acid sequence and (2) a pac site. The nucleic acid sequence and C1-regulated promoter sequence can be heterologous. In addition, the phage and phage capsids can contain a nucleic acid with any of the sequences described herein (e.g., nucleic acid encoding a Bof polypeptide, a C1 polypeptide, or a Coi polypeptide).

Examples of phage include, but are not limited to, bacteriophage P1 and variants thereof, phiX174 and variants thereof, and bacteriophage that are specific for particular strains of bacteria, such as, for example, Pseudomonas aeruginosa. Contemplated bacteriophage include, but are not limited to, phage with genomes consisting of ssDNA, dsDNA, ssRNA, and dsRNA. The bacteriophage of the instant invention include, but are not limited to, tailed, filamentous, polyhedral, and pleomorphic phage. An extensive list of contemplated phage can be found on the World Wide Web at phage.org/names.htm.

For example, considering the phage from the family Tectiviridae, this family of bacteriophage produces an icosahedral capsid with inner lipoprotein vesicle and a linear dsDNA, “tail” produced for DNA injection. Susceptible hosts and the appropriate phages are listed in this website. The tectiviridae family of phage has characteristics that may be exploited with the invention described here. Specific phages where information is available are hyperlinked (http://www.res.bbsrc.ac.uk/mirror/auz/ICTVdB/68010001.htm) to that information making it a useful tool to skilled workers. Contemplated bacterial species and the corresponding phage include, but are not limited to, the following:

| Bacterial Species | Phage |
|---|---|
| Alicyclobacillus | A, fNS11. |
| Bacillus | AP50, AP50-04, AP50-11, AP50-23, |
| | AP50-26, AP50-27, Bam35 |
| Enterobacteria-Pseudomonas | L172, PRD1, PR3, PR4, PR5, PR772 |
| Thermus | P37-14 |

Filamentous phage encompasses a group of bacteriophages that are able to infect a variety of Gram-negative bacteria through interaction with the tip of the F pilus. Well known filamentous phages include M13, fl, and fd. The genomes of these phage are single-stranded DNA, but replicate through a double-stranded form. Phage particles are assembled in the bacteria and extruded into the media. Because the bacteria continue to grow and divide, albeit at a slower rate than uninfected cells, relatively high titers of phage are obtained. Moreover, replication and assembly appear to be unaffected by the size of the genome. As a consequence of their structure and life cycle, the filamentous phage have become a valuable addition in the arsenal of molecular biology tools.

Further development of filamentous phage systems have led to the development of cloning vectors, called phagemids, that combine features of plasmids and phages.

Phagemids contain an origin of replication and packaging signal of the filamentous phage, as well as a plasmid origin of replication. Other elements that are useful for cloning and/or expression of foreign nucleic acid molecules are generally also present. Such elements include, without limitation, selectable genes, multiple cloning site, primer sequences. The phagemids may be replicated as for other plasmids and may be packaged into phage particles upon rescue by a helper filamentous phage. As used herein, “filamentous phage particles” refers to particles containing either a phage genome or a phagemid genome. The particles may contain other molecules in addition to filamentous capsid proteins.

Filamentous phages have also been developed as a system for displaying proteins and peptides on the surface of the phage particle. By insertion of nucleic acid molecules into genes for phage capsid proteins, fusion proteins are produced that are assembled into the capsid (Smith, Science 228, 1315, 1985; U.S. Pat. No. 5,223,409). As a result, the foreign protein or peptide is displayed on the surface of the phage particle. Methods and techniques for phage display are well known in the art (see also, Kay et al., Phage Display of Peptides and Proteins: A Laboratory Manual, Academic Press, San Diego, 1996).

Filamentous phage vectors generally fall into two categories: phage genome and phagemids. Either type of vector may be used within the context of the invention. Many such commercial vectors are available. For example, the pEGFP vector series (Clontech; Palo Alto, Calif.), M13 mp vectors (Pharmacia Biotech, Sweden), pCANTAB SE (Phammacia Biotech), pBluescript series (Stratagene Cloning Systems, La Jolla, Calif.); pBBR122 (Mobitec); and others may be used.

Other vectors are available in the scientific community (see e.g., Smith, in Vectors: A Survey of Molecular Cloning Vectors and their Uses, Rodriquez and Denhardt, eds., Butterworth, Boston, pp 61-84, 1988) or may be constructed using standard methods (Sambrook et al., Molecular Biology: A Laboratory Approach, Cold Spring Harbor, N.Y., 1989; Ausubel et al., Current Protocols in Molecular Biology, Greene Publishing, N.Y., 1994) guided by the principles discussed below.

At a minimum, for use in the present invention, the vector must accept a cassette containing a promoter and a gene of interest in operative linkage. Any promoter that is active in the cells to be transfected can be used. The vector can have a phage origin of replication and a packaging signal for assembling the vector DNA with the capsid proteins.

Other elements may be incorporated into the construct. In some embodiments, the construct includes a transcription terminator sequence, including a polyadenylation sequence, splice donor, and acceptor sites, and an enhancer. Other elements useful for expression and maintenance of the construct in mammalian cells or other eukaryotic cells may also be incorporated (e.g., origin of replication). Because the constructs are conveniently produced in bacterial cells, elements that are necessary or enhance propagation in bacteria are incorporated. Such elements include an origin of replication, selectable marker and the like.

The promoter that controls expression of the gene of interest should be active or activatable in the targeted cell. Within the present invention, the targeted cell may be bacterial, fungal, mammalian, avian, plant, and the like. Applications of the invention include transfection or transformation of bacterial, fungal or mammalian cells, including human, canine, feline, equine, and the like. The choice of the promoter will depend in part upon the targeted cell type and the degree or type of control desired. Promoters that are suitable within the context of the invention include, without limitation, constitutive, inducible, tissue specific, cell type specific, temporal specific, or event-specific, such as temperature sensitive promoters, for example.

4. Transformation

Transformation of cells with a recombinant DNA molecule of the invention is accomplished by well known methods that typically depend on the type of vector used and host system employed. With regard to transformation of prokaryotic host cells, electroporation and salt treatment methods are typically employed, see, for example, Cohen et al. Proceedings of the National Academy of Science USA, Vol. 69, no. 8 (1972) pp. 2110-2114; and Maniatis et al. Molecular Cloning: A Laboratory Mammal. Cold Spring Harbor, N.Y. Cold Spring Harbor Laboratory Press, 1982). With regard to transformation of vertebrate cells with vectors containing recombinant DNAs, electroporation, cationic lipid or salt treatment methods are typically employed, see, for example, Graham et al. Virology, Vol. 52, no. 2 (1973) pp. 456-467; and Wigler et al. Proceedings of the National Academy of Science USA, Vol. 76 (1979) pp. 1373-1376.

Additional protocols for inducing artificial competence in prokaryotic hosts such as prolonged incubation with calcium chloride, treatment of bacteria with dimethyl sulfoxide, hexaminecobalt, and dithiothreitol in the presence of cations or addition of polyethylene glycol can be used. Additional techniques include phage transduction, conjugational mating, and mobilization of plasmids within biofilm.

Successfully transformed cells, i.e., cells that contain a recombinant DNA molecule of the invention, can be identified by well known techniques including the selection for a selectable marker. For example, cells resulting from the introduction of an recombinant DNA of the invention can be cloned to produce single colonies. Cells from those colonies can be harvested and lysed, and their DNA content examined for the presence of the recombinant DNA using a method such as that described by Southern, Journal of Molecular Biology, Vol. 98, no. 3 (1975) pp. 503-517; or Berent et al. Biotechnic and Histochemistry, Vol. 3 (1985) pp. 208; or the proteins produced from the cell assayed via an immunological method.

5. Bacteriophage Used as Delivery Vehicles

Several mechanisms of gene transfer have been identified in bacteria such as conjugation, transformation, vesicle-mediated uptake, and transduction. The mechanism by which DNA is encapsulated into phage particles to enable use the bacteriophage as a delivery vehicle. During the infection process, transducing phage are capable of delivering host genetic material including resident phage, transposable elements, plasmids, and chromosomal DNA by several distinct mechanisms. For example, plasmid DNA encapsulation into P1 phage particles occurs when nucleotide sequences resembling those used by the phage for packaging of its own DNA (the pac site) are recognized and used for encapsulation of phage-genome sized segments. A plasmid that contains a pac site and can attain a size that completely fills a P1 phage head can therefore be packaged by the bacteriophage P1. Other Gram-negative and Gram-positive phage, for example P22 and phi11, are also capable of transducing plasmids which contain a P22 or phi11 pac site (Novick, R. P., I. Edelman, and S. Lofdal. 1986. Small Staphylococcus aureus plasmids are transduced as linear multimers that are formed and resolved by replicative process. JMB 192:209-220; Schmidt, C., and H. Schmieger. 1984. Selective transduction of recombinant plasmids with cloned pac sites by Salmonella phage P22. Molecular and General Genetics 196:123-128). In addition, the phage delivery systems disclosed in PCT publications WO 98/24925, WO 99/67400, WO 00/61804, and WO 01/79524 can be used in connection with the invention.

6. Recombinant Expression

The invention provides methods for expressing a gene of interest using nucleic acids described herein. In general terms, the production of a recombinant form of a polypeptide typically involves the following steps. First, a nucleic acid molecule is obtained that encodes a polypeptide of interest. If the sequence is uninterrupted by introns, it is directly suitable for expression in any host. The nucleic acid molecule is then preferably placed in operable linkage with suitable control sequences, as described herein, to form an expression unit containing the open reading frame. The expression unit is used to transform a suitable host and the transformed host is cultured under conditions that allow the production of the recombinant protein. Optionally, the recombinant polypeptide is isolated from the medium or from the cells; recovery and purification of the polypeptide may not be necessary in instances where some impurities may be tolerated, particularly if the polypeptide of interest is a membrane bound receptor. Each of the foregoing steps can be done in a variety of ways. For example, the desired coding sequences can be obtained from genomic fragments and used directly in appropriate hosts. The construction of expression vectors that are operable in a variety of hosts is accomplished using appropriate replicons and control sequences, as set forth herein. The control sequences, expression vectors, and transformation methods are dependent on the type of host cell used to express the gene. Suitable restriction sites can, if not normally available, be added to the ends of the coding sequence so as to provide an excisable gene to insert into these vectors.

7. Kits

The invention provides nucleic acid constructs and vectors formulated as compositions for therapeutic, diagnostic, or research purposes. Such formulations can be in a kit or container, packaged with instructions pertaining to controlled expression of a desired nucleic acid(s) of interest or the transformation or transfection of a cell of interest.

Formulations or compositions of the invention can be packaged together with, or included in, a kit with instructions or a package insert referring to the nucleic acid constructs and/or bacteriophage of the invention. For instance, such instructions or package inserts may address recommended storage conditions, such as time, temperature, and light. Such instructions or package inserts may also address the particular advantages of the nucleic acid constructs and bacteriophage of the invention, such as the ease of storage for formulations that may require use in the field, outside of controlled hospital, clinic, laboratory, or office conditions.

8. Genetic Approaches

The methods and materials provided herein can be used for many genetic approaches including (1) the construction of strains, (2) the heterologous expression of genes and proteins, and (3) the analysis of endogenous gene expression. One important advantage of a phage delivery system is, in contrast to transformation, phage infection normally occurs at high frequency in hosts competent for that phage. Low transformation efficiency of many bacteria has prevented the introduction of a gene library into these bacteria for direct complementation. In addition to using this procedure for the generation of recombinant bacteria, it is also possible to construct libraries (e.g., genomic libraries) in the phagemid vector. After obtaining transformants in E. coli, the library can be pooled and infected en masse with P1 phage, generating an entire packaged library. This can be used to transfect any P1-sensitive host in vitro and in vivo.

Transduction by bacteriophage has been reported in marine and freshwater aquatic habitats and in soil (Miller, Scientific American 47:67-71 (1998) and Zeph et al., Appl. Environ. Microbiol., 54:1731-1737 (1988)). The P1 delivery system is helpful in addressing questions concerning the fate of genetically engineered organisms released into these environments, the transfer by transduction of DNA to indigenous organisms, and detection of pathogenic bacteria. In this regard, genetically modified bacteriophage have been developed for transduction of bioluminescence and identification tags to pathogenic bacteria (Daniell et al., J. Appl. Microbiol., 88:860-869 (2000); Favrin et al., Appl. Environ. Microbiol 67:217-224 (2001); and Waddell and Poppe, FEMS Microbiol. Lett., 182:285-289 (2000)).

Clinically important microorganisms that are rapidly developing resistance to available antimicrobials include Gram-negative bacteria that cause urinary tract infections (Gupta et al., JAMA, 281:736-738 (1999)), foodborne infections (Glynn et al., N. Engl. J. Med., 338:1333-1338 (1998)), bloodstream infections (Pittet and Wenzel, Arch. Intern. Med., 155:1177-1184 (1995)), and infections transmitted in health care settings (Richard et al., J. Infect. Dis., 170:377-383 (1994) and Wiener et al., JAMA, 281:517-523 (1999)). Besides being a valuable tool for delivering DNA in vitro, this technology provides the opportunity for targeting bacterial cells in vivo. This system (Phagemune™) can be used as a delivery vehicle for oral vaccines if the natural enteric flora of the gastrointestinal tract was targeted. In this approach, P1 phage can deliver phagemids engineered to express pathogen-specific immunogenic epitopes on the surface of the bacteria (Zuercher et al., Eur. J. Immunol., 30:128-135 (2000)). Alternatively, phage delivered vectors can direct oral bacteria to secrete salivary histatin or other antimicrobial peptides (Hancock and Capple, Antimicrob. Agents Chemother., 43:1317-1323 (1999). This approach can be useful in the management of mucosal candidiasis and development of antimicrobial therapies.

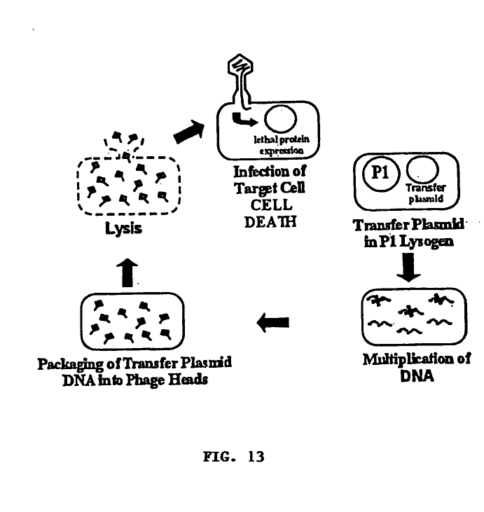

Another approach termed lethal agent delivery system, LADS™, also can utilize a bacteriophage based in vivo packaging system to create a targeted phage head, which acts as a molecular syringe, capable of delivering naturally occurring molecules with bactericidal activity to drug resistant bacteria (FIG. 13). LADS™ includes of a transfer plasmid carrying the genes encoding the antimicrobial agents, a plasmid origin of replication, the origin of replication of the bacteriophage, and a packaging site that will insure that the nucleic acid is loaded into the phage head. In one embodiment, the transfer plasmid can be maintained in a bacteriophage lysogen which is unable to package its own DNA. However, the defective lysogen can provide all the replication factors needed to activate the bacteriophage origin of replication on the transfer plasmid and all the structural components necessary to form mature virions containing the antimicrobial agent. The lysogen also can carry a temperature-sensitive repressor mutation so that LADS™. production is controlled by induction of the lysogen by a temperature shift, resulting in multiplication of DNA, packaging of the transfer plasmid into P1 phage heads, and lysis of the production strain. The virions or antimicrobial agents can be harvested and used to deliver the transfer plasmid to the pathogen. The phage head contains multiple copies of transfer vector DNA and can be targeted to pathogenic bacteria by natural receptor mediated mechanisms. Upon delivery, plasmid DNA recircularizes and expression of the lethal agent under the control of environmental, virulence-regulated, or species-specific promoters results in rapid cell death. Similar strategies can be directed against Gram-positive organisms. Lethal agents delivered by LADS™ can be naturally occurring lethal genes associated with plasmids, bacteriophage, or bacterial chromosomes such as doc, chpBK, and gef A multitude of these genes exists (see, e.g., PCT publications WO 98/24925, WO 99/67400, WO 00/61804, and WO 01/79524). The lethality of these methods and materials were demonstrated in E. coli. In fact, doc, derived from bacteriophage P1 was experimentally determined to be lethal in E. coli and is either lethal or bacteriostatic in P. aeruginosa, S. aureus and E. faecalis.

LADS™ offers many unprecedented advantages over conventional antimicrobial therapy including: (1) the preparation would bypass any de novo built in drug resistance, which sophisticated warfare agents will be expected to have; (2) it is not presently feasible to counteract the lethal agents delivered to a naive prokaryotic cell; (3) should the weaponized bacteria have resistance against one of the lethal agents, the LADS™ preparation could be engineered such that several lethal agents are be delivered simultaneously in order to address the issue; (4) custom design of the bacteriophage construct can be readily tailored to different families of organisms; (5) the phage is a non-replicating, artificial construct easy to assemble, and as such is less likely to engender questions relative to human use; (6) the preparation can be an inhalant that can be lyophilized and stable over long-term storage conditions; (7) use of an inhalant would reduce the immunogenicity of the bacteriophage preparations as opposed to its use parenterally; (8) animal test systems exist allowing a measured, incremental approach to determine efficacy in the field; and (9) mathematical and practical testing can be accomplished that provide for a formula for using any LADS agent in the patient setting. Therefore, with the combination of this delivery approach and an aggressive mechanism for quickly inactivating bacterial cells, the timely defeat of bio-threat agents within the body can be accomplished before they have an opportunity to cause disease. The pseudoviron can be suitable for delivery to any individual at risk through any number of mechanisms from injection to inhalation.

The invention will be further described in the following examples, which do not limit the scope of the invention described in the claims.

### EXAMPLES

### Example 1

### Vectors for Regulated Expression

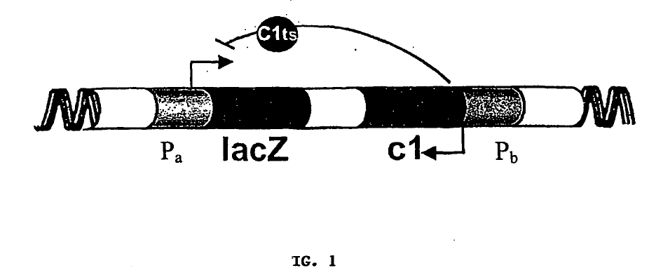

The following components were used to create vectors (FIGS. 1-3) for regulating expression of nucleic acid: an Op72 promoter, which is a C1-regulated promoter (Schaefer and Hays, J. Bacteriology, 173(20):6469-74 (1991)); nucleic acid encoding a temperature sensitive C1 repressor polypeptide, which can bind to Op72 and prevent transcription and which harbors a temperature sensitive mutation (Rosner, Virology, 49:679-689 (1972)); nucleic acid encoding a Bof modulator polypeptide, which can aid binding of a C1 repressor polypeptide to the Op72 promoter (Vellman et al., J. Biol. Chem., 265(30):18511-7 (1992) and Vellman et al., J. Biol. Chem., 267(17):12174-81 (1990)); nucleic acid encoding a Coi polypeptide, which is a C1 inactivator polypeptide (Baumstark et al., Virology 179:217-227 (1990); Heinzel et al., J. Biol. Chem., 265(29):17928-34 (1990); Heinzel et al., J. Biol. Chem., 267(6):4183-8 (1992)); nucleic acid encoding a LacI repressor polypeptide, which provides a two-component system and aids induced activity (Backman and Ptashne, Cell, 13:65-71 (1978) and Stark, Gene 51(2-3):255-67 (1987)); and transcriptional terminators TL₁₇, rrnBT1, and rrnBT2, which can stop transcriptional readthrough from cryptic promoters and can prevent runaway transcription (Brosius et al., Plasmid 6(1):112-8 (1981) and Wright et al., EMBO Journal 11(5):1957-64 (1992)). In addition, a pBBR122 vector, which is a broad host range Gram negative vector available from MoBiTec, was used.

DNA manipulations were performed as described by Sambrook et al., (Molecular Cloning: a Laboratory Manual, 2nd ed. Cold Spring Harbor, N.Y.: Cold Spring Harbor Laboratory (1989)) and the recommendations of the enzyme manufacturers. The vectors were created by modifying the pBBR122 (pBBR122 supplied by MoBiTec, originally purchased from Bio101 (see, World Wide Web at bio101.com; catalog number 5300-300); Antoine and Locht, Mol. Microbiol. 6(13):1785-99 (1992) and Kovach et al., Biotechniques 16(5):800-2 (1994)) vector in the following ways. To facilitate selection in P. aeruginosa, the β-lactamase gene including the upstream promoter region from pBluescript IISK+ (Stratagene) was amplified by PCR (5′ primer: 5′-CGCTTACAATTTAGGTGGCAC, SEQ ID NO:14; 3′ primer: 5′-AACTTGGTCTGACAGTTACC, SEQ ID NO:15) and subcloned into the ScaI site of pBBR122. To increase the number of restriction sites available for subcloning, the multiple cloning site (MCS) from pBluescript IISK+ was amplified by PCR using T3 and T7 primers and sublconed into the blunted EcoRI site of pBBR122. To stop read-through from cryptic promoters into the 5′ end of the expression cassette, ribosomal terminators rrnBT1 and rrnBT2 (Brosius et al., Plasmid 6(1):112-8 (1981)) and ribosomal terminators TL₁₇ (Wright et al., EMBO Journal 11(5):1957-64 (1992)) were cloned into the Sac11 and Sac1 sites, respectively, while the TL₁₇ terminator sequence was also subcloned into the 3′ end of the expression cassette (Kpn1 site) to stop runaway transcription. The lacZ gene was amplified by PCR using pMC1871 (Pharmacia) as template with a 5′ primer (5′-ATTATAGGATCCGGAGGTGTAGTATGGTCGTTTTACAACGTCGTGAC; SEQ BD NO:16) and a 3′ primer (5′-ATTTATGTCGACTCCCCCCTGCCCGGTTAT; SEQ ID NO:17), which contain BamH1 and Sal1 restriction sites, respectively, for cloning into the respective sites of pBBR122. The 5′ primer also contains a ribosomal binding site (RBS) to initiate translation. The C1-regulated promoters (Table 1) were obtained by annealing complementary oligos and cloned upstream of the LacZ gene into the blunted BamH1 site of pBBR122.

TABLE 1
Topography and sequence of C1-regulated promoters.

Op72 (SEQ ID NO:18):
TATATTGCTCTAATAAATTTATTAGTGTAATATCGCCTCAATG
ATATAACGAGATTATTTAAATAATCACATTATAGCGGAGTTAC
AP (SEQ ID NO:19):
AGCTTTGACAATTGCTCTAATAAATTTTATAATTGCCGCCCAT
TCGAAACTGTTAACGAGATTATTTAAAATATTAACGGCGGGTA

The Op72 promoter sequence from bacteriophage P1 contains two partially overlapping C1 operators (Op72a, top strand, 5′-ATTGCTCTAATAAATTT (SEQ ID NO:20); and Op72b, bottom strand, 5′-ATTACACTAATAAATTT (SEQ ID NO:21). The underlined sequences illustrate the C1-repressor polypeptide binding sites. Op72a matches the 17 bp consensus of 14 C1-controlled operators, while Op72b deviates from the consensus by two nucleotides (bolded-double underlined). The Op72 promoter exhibits a high level of expression even though it differs markedly from the E. coli consensus −10/−35 hexamers. The proposed −10 and −35 promoter elements are shown in bold. The artificial promoter (AP) contains a consensus C1-operator site (underlined) flanked by consensus −10/−35 hexamers (bold).

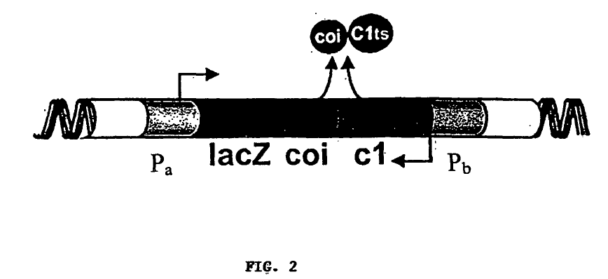

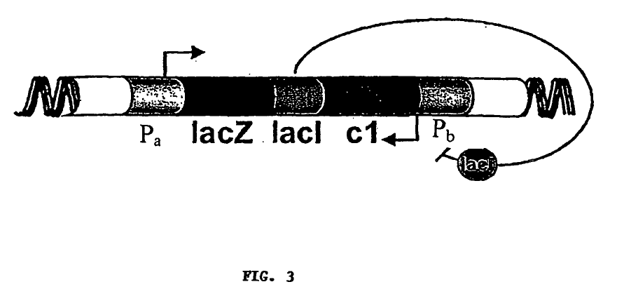

Nucleic acid encoding a Bof polypeptide was PCR amplified (5′ primer: 5′-GAATTCGCGACGCTCTACAGCC, SEQ ID NO:22; and 3′ primer: 5′-GAATTCTCGGTGAGCAAACAGCCAT, SEQ ID NO:23) from a thermosensitive mutant of P1 (Rosner, Virology, 49:679-689 (1972)) and cloned into the EcoR1 site of pACYC, while nucleic acid encoding C1 polypeptide was PCR amplified (5′ primer: 5′-GAATTCGGAGGAGGATCAATGATAAATTATG, SEQ ID NO:24; and 3′ primer: 5′-AAGCTTCTATTGCGCGCTTTCGGGGTTGTCG, SEQ ID NO:25) from the same template and cloned into the Sca1 site of pACYC. The c1.bof tandem was then PCR amplified (5′ primer: 5′-GAATTCGGAGGAGGATCAATGATAAATTATG, SEQ ID NO:26; and 3′ primer: 5′-GCATGCGGTGAGCAAACAGCCAT, SEQ ID NO:27) and cloned into a blunted Xho1 site of pBluescript IISK+. The LacI-regulated promoter (5′-AATTGACATGTGAGCGGATAACAATATAATGTGTGGAAGCT, SEQ ID NO:28) was cloned upstream of the c1 sequence in the blunted Kpn1 site thereby controlling C1 polypeptide expression. Where indicated, a nucleic acid sequence encoding a Coi polypeptide was PCR amplified (5′ primer: 5′-AGTCGAGTCGACGGAGGTGAATTATGGCTTTCATTCCACC, SEQ ID NO:29; and 3′ primer: 5′-AGTCGTGTCGACTTATTGTGAGTCTGGCTGG, SEQ ID NO:30) using P1 as template and cloned into the Sal1 sites of pBluescript IISK+ in the opposite orientation relative to the C1 polypeptide encoding sequence (FIG. 2). Similarly, the lacI gene was PCR amplified (5′ primer: 5′-CGAATTGGATCCGGAGGTGGAATGTGAAACCAGTAACG, SEQ D NO:30; and 3′ primer: 5′-TCGGCGGAATTCCTAATGAGTGAGCTAACT, SEQ D NO:31) from DH5a and cloned in the same sites and orientation as the coi sequence. The promoter-c1.bof fragment was then PCR amplified using T7 and the 3′ primer for bof, and cloned into the blunted Sal1 site of the pBBR122 expression vector in the opposite orientation relative to the lacZ sequence (FIG. 3).

This example describes the construction of broad host range vectors containing temperature sensitive C1-regulated promoters for controlling expression of genes in bacteria such as Gram-negative bacteria. As demonstrated herein, the constructs control expression in E. coli, P. aeruginosa, Klebsiella pneumoniae, and Shigella flexneri.

### Example 2

### Transformation Using Bacteriophage

The broad host range transducing bacteriophage P1 was used to deliver phagemids to a variety of clinically relevant Gram-negative species. All phagemids contain a P1 pac initiation site to package the vector, a P1 lytic replicon to generate concatemeric DNA, an origin of replication, and an antibiotic-resistance determinant to select bacterial clones containing the recircularized phagemid. P1 Phage available include Plkc (ATCC 25404-B1) and P1Cm c1ts100 (Rosner, Virology, 49:679-689 (1972)). Phagemid components included a Lytic replicon isolated from P1Cm c1ts100 (Hansen, J. Mol. Biol., 207(1):135-49 (1989); Heinrich et al., Nucleic Acids Research 23(9):1468-74 (1995); and Sternberg and Cohen, J. Mol. Biol., 207(1):111-33 (1989)) for rolling circle replication and a Pac site isolated from P1Cm clts100 (Stemberg and Coulby, 194(3):453-68 (1987)) for initiating packaging.

The following phagemids were constructed: P1pSK with an ampicillin antibiotic resistance marker and ColE1 plasmid origin in the parent vector pBluescript (Stratagene Ltd.); P1pBBR122 with a kanamycin resistance marker and broad host range plasmid origin in the parent vector pBBR122; P1pBBR122-T with a kanamycin resistance marker and broad host range plasmid origin in the parent vector P1pBBR122 with the addition of TL₁₇ terminators; P1pBBR122-bla with an ampicillin resistance marker, a kanamycin resistance marker, and a broad host range plasmid origin in the parent vector P1pBBR122.

DNA manipulations were performed as described by Sambrook et al., (Molecular Cloning: a Laboratory Manual, 2nd ed. Cold Spring Harbor, N.Y.: Cold Spring Harbor Laboratory (1989)) and the recommendations of the enzyme manufacturers. The following section describes construction of P1pBBR122-T.

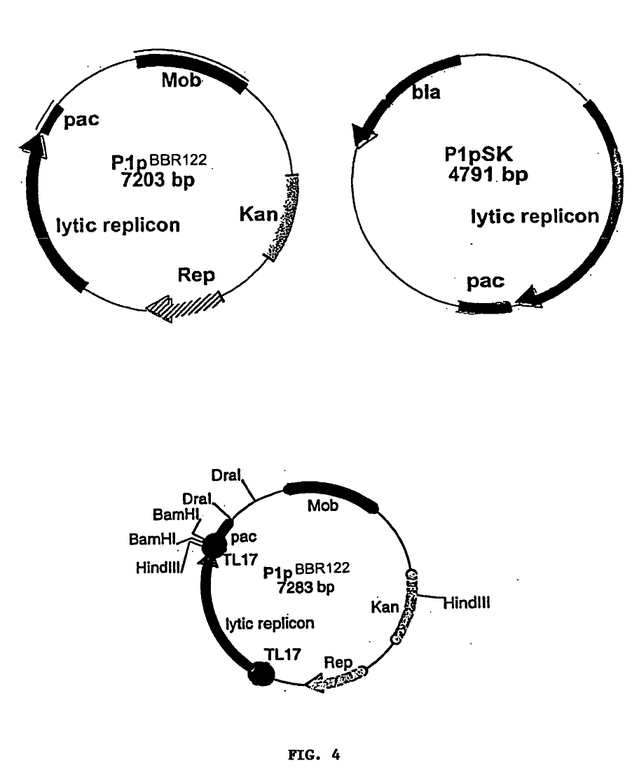

To construct a single vector capable of delivering DNA to a wide range of bacterial species, a phagemid was constructed containing all the essential signals for P1 packaging, a selectable marker for transfer detection, and a broad host range origin of replication (P1pBBR122-T, FIG. 4). The parent plasmid, pBBR122, is capable of replicating at medium copy number in at least 26 Gram-negative species and was stably maintained by selective pressure in all Gram-negative organisms tested so far (MoBiTec, LLC). The phagemid P1pBBR122-T was compatible with plasmids containing the ColE1 or p15A origins of replication and incompatibility tests demonstrated that the parent vector is not a member of the broad host range IncP, IncQ, or IncW groups (Antoine and Locht, Mol. Microbiol., 6(13):1785-99R (1992)). This is particularly relevant for transfer of the phagemid to clinical and environmental isolates since the majority of such strains may harbor native plasmids.

Nucleic acid encoding polypeptides involved in mobilization (mob), replication (rep), and kanamycin resistance (kan) were derived from the broad host range cloning vector pBBR122. The nucleic acid encoding the ampicillin resistance marker (bla) was derived from pBluescript II SK+. Sequences originating from the P1 bacteriophage included the packaging site (pac) and lytic replicon. The elements necessary for packaging into P1 phage capsids were inserted into pBBR122. These elements included the P1 lytic replicon and minimal pac site. The lytic replicon contains the C1 repressor-controlled P53 promoter, the promoter P53 antisense, the kilA genes, and the repL genes. The KiIA polypeptide is not essential for replicon function, but is lethal to the bacterial cell. Thus, the kilA gene was inactivated by an in-frame deletion resulting in a polypeptide 52 percent of the original size. During the late stages of the phage life cycle, the lytic replicon initiates a rolling circle mode of replication that generates concatemeric DNA, which is the substrate for packaging. Packaging is initiated when phage-encoded polypeptide recognize and cleave the unique pac site. The DNA is then brought into the empty P1 phage head, and packaging proceeds unidirectionally until the head is full. Since the P1 phage head can package ˜110 kb of DNA (Yarmolinsky and Stemberg, Bacteriophage P1, p. 291-438. In Calender, R. (ed), The bacteriophages. vol. 1. Plenum Publishing Corp, New York (1988)), fragments as large as 100 kb can be cloned and delivered by this system.

### Example 3

### Production of Phagemid-Containing Virions

The phagemid was maintained in a P1 lysogen that provided (1) all the replication factors needed to activate the lytic cycle and (2) all the structural components to form mature viral particles. The P1 lysogen also carried the c1.100 temperature-sensitive repressor mutation. This permitted rapid prophage induction by shifting the temperature of an exponentially growing lysogenic culture from 32° C. to 42° C. Induction of the lysogen by temperature shift resulted in multiplication of DNA, packaging of the phagemid into P1 phage heads, and lysis of the production strain. Lysates typically contained 80 percent wild type P1 and 20 percent phagemid particles, and were used to infect P1 sensitive strains.

To construct the P1 delivery vector, the signals necessary for packaging by the phage P1 were inserted into the cloning vector pBluescript II SK+. The P1 packaging site (pac) flanked by XbaI and BamHI restriction sites (shown in bold) was first produced by PCR using two primers (5′-GACAGCCTCTAGACAAATAAGCCAGTCAGGAAGCC, SEQ ID NO:32; and 5′-CGTACCGGGATCCAACGTTATCTATCAGGTAATCGCC, SEQ ID NO:33). The lytic replicon was generated by fusion of two PCR generated fragments resulting in a 52 percent in frame deletion of kilA. The kilA C-terminus and RepL gene was PCR amplified with flanking XhoI and HindIII sites using two primers (5′-ACCGTCCTCGAGACAAGCAATGGAAGCAGGATTTCTTTCACG, SEQ ID NO:34; and 5′-CGTCTCAAGCTTAGCCACTTATTGTTAGGTAGAATTGTCCG, SEQ ID NO:35). The DNA fragment containing the P53 promoter, P53 antisense promoter, and N-terminus of kilA was PCR amplified with XhoI containing primers (5′-GTCACACTCGAGCTGGCAGGTTTCTGAGCAGATCG, SEQ ID NO:36; and 5′-GTGGCACTCGAGGAACGAAACTATGCAATTCTGC, SEQ ID NO:37). The P1 elements were then PCR amplified as a cassette using the NcoI containing primers (5′-GTGACACCATGGCTGGCAGGTTTCTGAGCAGATCG, SEQ ID NO:38; and 5′-CGACACCCATGGTCTAGACAAATAAGCCAGTCAGGAAGC, SEQ ID NO:39) and inserted into the unique NcoI site of the broad host range vector pBBR122 (MoBiTec, LLC). In order to isolate the lytic replicon from transcriptional readthrough, the TL₁₇ terminator sequence was blunted into the unique BamHI and ScaI sites of P pBBR122 to generate P1pBBR122-T. To facilitate detection of phagemid transduction in P. aeruginosa, the ampicillin-resistance gene including its putative promoter was amplified (5′-CGCTTACAATTTAGGTGGCAC, SEQ ID NO:40; and 5′-AACTTGGTCTGACAGTTACC, SEQ ID NO:41) using PCR from pBluescript II SK⁺ and blunted into the DraI site of P1pBBR122-T.

### Example 4

### Thermal Induction of P1Cm c1ts100 Lysogens Harboring Plasmid P1pBBR122-T

The lysogen was grown at 30° C. in LC medium until OD₄₅₀ reached 1.0 at which time the culture was shifted to a 42° C. water bath and aerated until lysis occurred (about 1 hour). Chloroform (1% v/v), DNase (10 μg/mL), and RNase (1 μg/mL) were added, and incubation was continued for an additional 30 minutes at 37° C. The phage stock was clarified by centrifugation at 2,500 g for 15 minutes and passed through a 0.2 μm membrane filter.

### Example 5

### Phagemid Delivery and Analysis

An overnight culture of the host strain was diluted in LB and grown to mid-exponential phase (OD₆₀₀ of 0.4). The cells were centrifuged at 2,500 g for 10 minutes at 4° C. and concentrated to an OD₆₀₀ of 2.0 (10⁸ cfu/mL) with LC medium. Phage (100 mL) was added at various multiplicity of infections (moi) and allowed to adsorb to the cells (100 μL) for 15 minutes at 32° C. LC medium containing 10 mM sodium citrate was added (800 μL), and cells were incubated at 32° C. for 45 minutes or 90 minutes to allow expression of antibiotic-resistance genes (kanamycin and carbenicillin, respectively). The infection was centrifuged at 7,000 g for 5 minutes and resuspended in 100 μL LC medium containing 10 mM sodium citrate. Transductants were detected by spotting 7.5 μL of 10-fold serial dilutions of the infection onto LB agar plates containing the appropriate selection. Plates were scored following overnight incubation at 32° C. No transductants were observed when 10⁷ viable bacteria were assayed on selective media in the absence of phage lysate. P1pBBR122-T was recovered from transduced cells by the alkaline lysis method (QIAprep miniprep kit, Qiagen Inc.). Table 2 summarizes the bacteria, plasmids, and phage used.

TABLE 2
Designation, characteristics, and origins of bacteria, plasmids, and phage used.

| Bacteria, plasmid, or | | Source or |
|---|---|---|
| phage | Description or Genotype | Resource |
| Bacteria | | |
| E. coli | | |
| C600 | thi-1 thr-1 leuB6lacY1 tonA21 supE44 | Promega |
| JM101 | [F traD36 proAB lacI^{q}ZDM15] D(lac-proAB) | NEB |
| | glnv thi | |
| DH5″ | F-N80dlacZDM15 D(lacZYA-argF) U169 | Gibco BRL |
| | endA1 recA1 hsdR17 deoR thi-1phoA supE44 | |
| | 1-gyrA96 relA1 | |
| JM109 | [F traD36 proAB lacI^{q}ZDM15] D(lac-proAB) | NEB |
| | glnv44 e14 gyrA96 recA1 relA1 endA1 thi | |
| | hsdR17 | |
| EC-1 | Urine clinical isolate, Ampicillin resistant | MUSC |
| EC-2 | Urine clinical isolate, Ampicillin sensitive | MUSC |
| P. aeruginosa | | |
| PAO1 | Clinical isolate | PGSC |
| PA-1 | Clinical isolate | MUSC |
| S. flexneri | Serotype 2b | ATCC 12022 |
| C. freundii | Produces restriction endonuclease CfrA1 | ATCC 8090 |
| K. pneumoniae | Wild-type | ATCC 10031 |
| Plasmids | | |
| pBluescript II SK⁺ | Cloning vector, ColE1 origin | Stratagene |
| pBBR122 | Cloning vector, Broad host range origin | MoBiTec |
| Phage | | |
| P1Cm c1.100 | Thermoinducible P1 Cm | (10) |

Abbreviations:
NEB, New England Biolabs, Ltd;
MUSC, Medical University of South Carolina, Department of Pathology and Laboratory Medicine;
PGSC, Pseudomonas Genetic Stock Center, East Carolina University;
ATCC, American Type Culture Collection;
Cm, chloramphenicol marker.

### Example 6

### Controlled Expression in Klebsiella pneumoniae and Shigella flexneri Using a Bacteriophage P1-Derived C1-Regulated Promoter System

Many regulated promoter systems were described for use in Escherichia coli. These systems include promoters regulated by LacI (Backman and Ptashne, Cell 13:65-71 (1978)), AraC (Guzman et al., J. Bacteriol., 177:4121-4130 (1995)), and TetR (Lutz and Bujard, Nucleic Acids Res. 25:1203-1210 (1997)), or combinations that can provide both low basal and high induced expression. Each system has shown utility with varying success in other bacteria such as Pseudomonas aeruginosa (Brunschwig and Darzins, Gene, 111:35-41 (1992)), Corynebacterium glutamicum (Ben-Samoun et al., FEMS Microbiol. Lett., 174:125-130 (1999)), Agrobacterium tumefaciens (Newman and Fuqua, Gene, 227:197-203 (1999)), and Xanthomonas campestris (Sukchawalit et al., FEMS. Microbiol. Lett., 181:217-223 (1999)). However, little or no data exists for a regulated promoter system in the medically important species Klebsiella pneumoniae (Kleiner et al., J. Gen. Microbiol., 134:1779-1784 (1988)) and Shigella flexneri. Klebsiella species cause about 8 percent of nosocomial infections in the United States and are commonly found both in humans and the environment (Podschun and Ullmann, Clin. Micro. Rev., 11:589-603 (1998)). In contrast, Shigella species, found mainly in humans, results in shigellosis which is characterized by cramps, fever, and dysentery (Acheson and Keusch, In M. J. Blaser, P. D. Smith, J. I. Ravdin, H. B. Greenberg, and R. L. Guerrent, (ed.) Infections of the gastrointestinal tract, New York, N.Y.: Raven Press Ltd. (1995)).

The temperate bacteriophage P1 can infect and lysogenize several enterobacterial species, including K. pneumoniae and S. dysenteriae (Murooka and Harada, Appl. Environ. Micro., 38:754-757 (1979) and Yarmolinsky and Sternberg, Bacteriophage P1. p. 291-438. In Calender, R. (ed), The bacteriophages. vol. 1. Plenum Publishing Corp, New York (1988)). Stable lysogeny is maintained by the action of the components of the tripartite immunity system (Heinrich et al., FEMS Microbiol. Rev., 17:121-126 (1995)). The C1 repressor polypeptide acts as a central regulator by binding to and negatively regulating promoter elements for a variety of genes (Citron et al., J. Biol. Chem., 264:3611-3617 (1989); Eliason and Sternberg, J. Mol. Biol., 198:281-293 (1987); Heinzel et al., J. Mol. Biol., 205:127-135 (1989); Heinzel et al., J. Biol. Chem., 265(29):17928-34 (1990); Lehnherr et al., J. Bacteriol., 174:6138-6144 (1992); Lehnherr et al., J. Bacteriol. 183:4105-4109 (2001); Velleman et al., PNAS, 84:5570-5574 (1987)).

The C1 asymmetric operator sites (consensus sequence ATTGCTCTAATAAATTT; SEQ ID NO:42) are widely dispersed over the P1 genome and are numbered according to their position on the P1 genetic map.

In this example, a temperature sensitive C1-regulated promoter engineered into a broad host range plasmid is provided for controlling gene expression in both K. pneumoniae and S. flexneri.

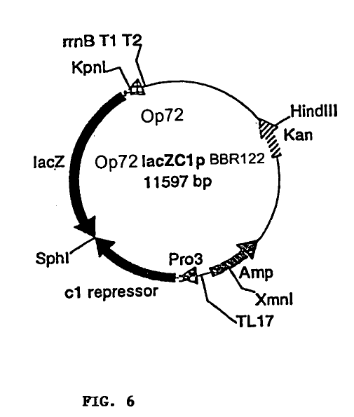

The lacZ reporter gene vectors were constructed in the broad host range Gram-negative plasmid pBBR122 (MoBiTec). The lacZ gene was placed under the transcriptional control of Op72 or AP (FIG. 5). The Op72 promoter is based on the promoter responsible for driving ban gene expression in bacteriophage P1 and is effectively repressed in E. coli in the presence of C1. It contains of two overlapping C1 operator sites, but lacks consensus E. coli −10 and −35 promoter elements. In contrast, the AP sequence contains a consensus C1 operator site flanked by consensus −10 and −35 promoter elements. To prevent read-through from cryptic promoters and ‘runaway’ transcription, the ribosomal terminators rrnBT1 and rrnBT2 were placed at the 5′end of the expression cassette, and the ribosomal terminator TL₁₇ was placed at the 3′ end (FIG. 6). To control gene expression, nucleic acid encoding a temperature sensitive C1 polypeptide from the thermoinducible bacteriophage P1Cm carrying the c1.100 mutation was PCR amplified and was placed under the transcriptional control of either (1) a promoter containing consensus E. coli −10 and −35 promoter elements (Pro3, FIG. 5) or (2) a promoter containing two mismatches from the consensus (Pro4, FIG. 5). These constructs were designed to provide differing amounts of the C1 repressor polypeptide. At the permissive temperature, C1 polypeptide binds to its operator site and prevents transcription from the gene of interest, while at the non-permissive temperature, C1 polypeptide is thermally unstable, thereby allowing transcription to proceed. Where indicated, the coi gene (Baumstark et al., Virology, 179:217-227 (1990)) from bacteriophage P1 was PCR amplified and placed 3′ of the lacZ gene to ensure full derepression from the promoters.

The following experiments were performed to determine whether the C1 polypeptide would be functional in Gram-negative bacteria such as K. pneumoniae and Shigella species. β-Gal expression under the control of either of the two C1-regulated promoters was examined at the permissive (31° C.) and non-permissive (42° C.) temperatures in S. flexneri ATCC 12022 (Table 3) which was transformed with the reporter plasmids as described previously (Lederberg and Cohen, J. Bacteriol., 119:1072-1074 (1974)). In the absence of C1 polypeptide, activity from both promoters was high with Op72 being stronger than AP. This suggested that promoter recognition elements, other than the consensus −10 and −35 hexamers were being efficiently recognized in S. flexneri. In the presence of C1 polypeptide and at the permissive temperature, β-Gal activity was significantly reduced from both promoters indicating that C1 polypeptide can efficiently repress expression. In particular, the basal expression of Op72 was extremely low as compared to AP (1 and 69 Miller units, respectively), which may be a reflection of the two overlapping C1 binding sites located within this promoter. The basal expression of Op72 was similar to the background activity levels displayed by the control strain carrying the plasmid containing the promoterless lacZ gene. This indicated that the promoter was completely repressed in the presence of C1 polypeptide. This level of repression is similar to the levels of repression observed in E. coli. Little difference was observed in the basal expression when C1 polypeptide was expressed from either a consensus promoter (Pro3) or a promoter with two mismatches in the conserved hexamers (Pro4).

TABLE 3
Basal and induced activities from lacZ fusions to
C1-regulated promoters in S. flexneri.

| | | Miller units | | |
|---|---|---|---|---|
| | C1 | Basal activity | Induced activity | Fold- |
| Construct | repressor | (31° C.) | (42° C.) | induction |
| Control | + | 2.9 | 3.1 | 1.1 |
| Op72lacZ | − | 924.9 | 932.9 | 1.0 |
| Op72lacZ | + | 0.9 | 90 | 100 |
| Op72lacZ* | + | 1.1 | 176.9 | 161 |
| APlacZ | − | 622.4 | 628.3 | 1.0 |
| APlacZ | + | 69.2 | 576.3 | 8.4 |
| Overnight cultures were diluted 1:100 and grown to about an OD₆₀₀ of 0.1 in LB medium at 31° C. The culture was then divided equally and incubated at 42° C. or 31° C. for 105 minutes prior to assaying for β-Gal activity (OD₆₀₀ of about 0.6). The control strain carried a plasmid containing c1 and a promoterless lacZ gene. β-Gal activity was | | | | |
| #measured according to Miller (Experiments in Molecular Genetics. Cold Spring Harbor Laboratory Press. Cold Spring Harbor, New York. (1972)) and samples (n = 3) assayed in triplicate (standard deviation < 5%). | | | | |

*denotes the Pro4 promoter driving C1 polypeptide expression.

To examine the levels of induction from both promoters, the cultures were incubated at the permissive temperature, divided equally, and shifted to the non-permissive temperature for 95 minutes to allow for expression of LacZ (Table 3). This resulted in a significant increase in P-Gal activity from both promoters, albeit for Op72 this still was below fully induced levels. Nevertheless, this represented up to 161-fold induction for Op72 depending on the expression signals for the promoter driving C1 polypeptide expression. The AP exhibited a much lower fold induction (8-fold) than Op72 primarily because of its leaky expression. However, the results indicated that a ts C1-regulated promoter can be effectively repressed to levels comparable to the control vectors yet give high levels of induced expression. This represents the first heterologous regulated promoter system for S. flexneri.

β-Gal expression under the control of either of the two C1-regulated promoters was examined at the permissive (31° C.) and non-permissive (42° C.) temperatures in K. pneumoniae ATCC 10031 (Table 4), which was transformed as described previously (Merrick et al., J. Gen. Microbiol., 133:2053-2057 (1987). As for S. flexneri, Op72 was stronger than AP and, in the presence of C1 polypeptide, exhibited extremely low levels of basal expression that were comparable to control vectors. These results indicate that the promoters are being efficiently recognized by the transcriptional machinery and that C1 polypeptide can effectively repress transcription. However, in contrast to S. flexneri, levels of induction were modest (4 to 27-fold). While still retaining low basal expression, highest levels of induction were achieved when the weaker promoter driving C1 polypeptide expression was utilized (5 and 58 Miller units, respectively). This suggests high induced expression cannot be achieved if the repressor molecule is overexpressed. To increase the levels of derepression at elevated temperatures, the level of available C1 polypeptide was controlled by cloning the coi gene 3′ of the lacZ sequences, thereby transcriptionally coupling its expression to LacZ expression. The coi gene encodes a C1 inactivator polypeptide (e.g., a Coi polypeptide) from bacteriophage P1 (Heinzel et al., J. Biol. Chem., 265(29):17928-34 (1990)), which exerts its antagonistic effect by forming a complex with the C1 repressor polypeptide. The addition of nucleic acid encoding a Coi polypeptide resulted in high levels of induced expression. However, while this resulted in 19-fold induction, the basal expression from this vector was also increased. Therefore, this construct may be more suitable when high levels of induced activity are desired. In summary, good regulation (27-fold) of β-Gal activity can be achieved in K. pneumoniae, and depending on the constructs utilized, can either yield low basal expression or fully induced activity.

TABLE 4
Basal and induced activities from lacZ fusions to
C1-regulated promoters in K. pneumoniae.

| | | Miller units | | |
|---|---|---|---|---|
| | | Basal activity | Induced activity | Fold- |
| Construct | C1 repressor | (31° C.) | (42° C.) | inducti [symbol] |
| Control | + | 3.2 | 4.1 | 1.3 |
| Op72lacZ | − | 409.9 | 536.0 | 1.3 |
| Op72lacZ | + | 1.4 | 5.2 | 3.7 |
| Op72lacZ* | + | 2.2 | 58.6 | 26.6 |
| Op72lacZcoi | + | 36.4 | 697.9 | 19 |
| APlacZ | − | 307.7 | 457.0 | 1.5 |
| APlacZ | + | 36.9 | 221.2 | 6.0 |

Overnight cultures were diluted 1:100 and grown to about an OD₆₀₀ of 0.1 in LB medium at 31° C. The culture was then divided equally and incubated at 42° C. or 31° C. for 75 minutes prior to assaying for β-Gal activity (OD₆₀₀ of about 0.6). The control strain carried a plasmid containing c1 and a promoterless lacZ gene. Values are averages of multiple cultures (n = 3) assayed in triplicate (standard deviation < 5%).
*denotes the Pro4 promoter driving C1 polypeptide expression.

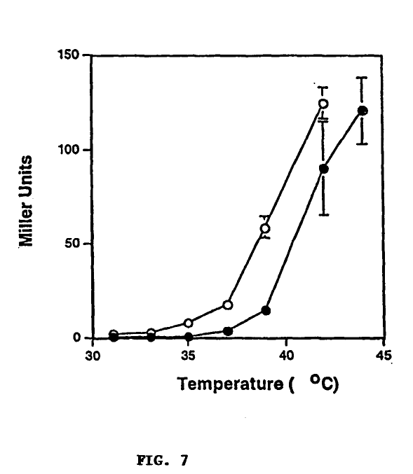

Another feature of a controlled expression construct is the ability to obtain different levels of expression by partial induction of the promoter. Therefore, to assess the ability to modulate expression using a temperature sensitive C1-regulated promoter, the extent of induction from Op72 at different temperatures was measured. The results indicated that it was possible to achieve partial induction of the promoter (FIG. 7). However, the ability to modulate activity was more pronounced in K. pneumoniae than in S. flexneri. For example, incubation at 37° C. and 39° C. for K. pneumoniae resulted in 15 percent and 50 percent of maximal induced activity, respectively. In contrast, this only represented 4 percent and 17 percent of maximal induced activity under the same conditions for S. flexneri. Maximal induction was achieved at 42° C. or higher, which is consistent with other temperature sensitive-regulated promoter systems (Remaut et al., Gene, 15:81-93 (1981).

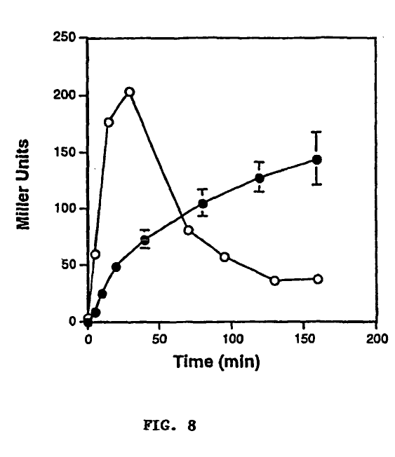

To examine the kinetics of induction from a temperature sensitive C1-regulated promoter, cultures were grown under repressing conditions and then induced at the elevated temperature (FIG. 8). At the indicated times, cultures were harvested and β-Gal activity was determined. For S. flexneri, activity ranged from 0.6 Miller units under repressed conditions to 144 units after 160 minutes under inducing conditions, which represented a 240-fold induction of β-Gal activity. In contrast, maximal induced activity was achieved after 30 minutes for K. pneumoniae, which corresponded to a 50-fold induction. This level of regulation is comparable to that achieved with the commonly used P_{tac} promoter in E. coli (Guzman et al., J. Bacteriol., 177:4121-4130 (1995)). In addition, incubation for longer time periods at the induced temperature resulted in a dramatic decrease in β-Gal activity, which may be due to instability of LacZ at elevated temperatures. Alternatively, the rapid decrease in activity may be a reflection of the detrimental effects of the elevated temperature to the cells physiology. However, as the cells were growing rapidly, this appears unlikely.

In summary, the temperature sensitive C₁-regulated promoter exhibited very low basal expression with the ratio of induction/repression up to 240-fold for S. flexneri and up to 50-fold for K. pneumoniae. These results indicate the usefulness of the expression system in S. flexneri and K. pneumoniae, which can provide new opportunities for controlled gene expression in enteric Gram-negative bacteria.

### Example 7

### Tight Regulation and Modulation via a C1-Regulated Promoter in Escherichia coli and Pseudomonas aeruginosa

Although the lactose repressor/isopropylthio-β-galactoside (IPTG) system employs many different promoters of varying strengths (P_{lac}, P_{tac}, Pₜᵣₚ), they are characterized as leaky (Stark, Gene, 51(2-3):255-67 (1987)) and are therefore not suitable when tight control is required such as when cloning toxic gene products. When tight control is required, the most frequently employed system is the arabinose PBAD promoter controlled by the AraC polypeptide (Guzman et al., J. Bacteriol., 177:4121-4130 (1995)). However, minimal media is required for optimal regulation, the promoter system is not suitable when overexpression of the polypeptide is required, and induction may reflect a population average of induced and uninduced cells (Siegele and Hu, PNAS, 94:8168-8172 (1997)). An alternative system utilizes the RNA polymerase promoter of phage T7 (Tabor and Richardson, PNAS, 82:1074-1078 (1985)). However, the production of lambda phage and infection of large scale cultures presents difficulties, while placement of the polymerase under the control of a lacI or araC promoter compromises the system (Wycuff and Matthews, Anal. Biochem., 277:67-73 (2000)). Fewer choices of regulated promoter systems with significantly less range exist for P. aeruginosa (Bagdasarian et al., Gene, 26:273-282 (1983) and Brunschwig and Darzins, Gene, 111:35-41 (1992)).

A temperature sensitive regulated promoter system in a broad-host range plasmid for use in E. coli and P. aeruginosa is provided herein. The repression, induction, and modulation of the temperature sensitive C1-regulated promoter driving expression of a gene of interest (e.g., lacZ) was examined using (1) a C1-regulated promoter derived from bacteriophage P1, Op72, and (2) an artificial promoter, AP.

The E. coli strains used for this experiment were DH5a (Gibco BRL), TB1, and ER1793 (New England Biolabs). Cultures were grown in LB supplemented as needed with the following antibiotics: ampicillin (100 μg/mL), kanamycin (50 μg/mL), tetracycline (50 μg/mL) for E. coli and carbenicillin (500 μg/mL) for P. aeruginosa. pBluescript IISK⁺ was obtained from Stratagene, pACYC184 from New England Biolabs, and the broad host-range vector pBBR122 was obtained from MoBiTec.

The pBBR122 vector was modified in the following ways. To facilitate selection in P. aeruginosa, the β-lactamase gene including the upstream promoter region from pBluescript IISK+ (Stratagene) was amplified by PCR as described in Example 1 and subcloned into the Sca1 site of pBBR122. To increase the number of restriction sites available for subcloning, the multiple cloning site (MCS) from pBluescript IISK+ was amplified by PCR using T3 and T7 primers and sublconed into the blunted EcoR1 site of pBBR122. To stop read-through from cryptic promoters into the 5′ end of the expression cassette, ribosomal terminators rrnBT1 and rrnBT2 (Brosius et al., Plasmid 6(1):112-8 (1981)) and ribosomal terminators TL₁₇ (Wright et al., EMBO Journal 11(5): 1957-64 (1992)) were cloned into the SacII and SacI sites, respectively, while the TL₁₇ terminator sequence was also subcloned into the 3′ end of the expression cassette (Kpn1 site) to stop runaway transcription. The lacZ gene was amplified by PCR using pMC1871 (Pharmacia) as template as described in Example 1 for cloning into pBBR122. The 5′ primer contained a RBS to initiate translation. The C1-regulated promoters, Op72 and AP, were obtained by annealing complementary oligos and cloned upstream of the LacZ gene into the blunted BamHI site of pBBR1221. Nucleic acid encoding a Bof polypeptide was PCR amplified (5′ primer: 5′-TCAGTAGAATTCGCGACGCTCTACAGCCA, SEQ ID NO:43; and 3′ primer: 5′-GCGGATGAATTCTCGGTGAGCAAACAGCCAT, SEQ ID NO:44) from a thermosensitive mutant of P1 (Rosner, Virology, 49:679-689 (1972)) and cloned into the EcoR1 site of pACYC184, while nucleic acid encoding C1 polypeptide was PCR amplified (5′ primer: 5′- CGCATGGAATTCGGAGGAGGATCAATGATAAATTATG, SEQ ID NO:45; and 3′ primer: 5′-GCAGCTAAGCTTCTATTGCGCGCTTTCGGGGTTGTCG, SEQ ID NO:46) from the same template and cloned into the Sca1 site of pACYC184. The c1.bof tandem was then PCR amplified as described in Example 1 and cloned into a blunted Xho1 site of pBluescript IISK+. The LacI-regulated promoter was cloned upstream of the c1 sequence in the blunted Kpn1 site as described in Example 1, thereby controlling C1 polypeptide expression.

Where indicated, a nucleic acid sequence encoding a LacI polypeptide was PCR amplified as described in Example 1 and cloned into the Sal1 sites of pbluescript IISK+ in the opposite orientation relative to the C1 polypeptide encoding sequence. The promoter-c1.bof.laci fragment was then PCR amplified using T7 and the 3′ primer for lacI, and cloned into the blunted Sal1 site of the pBBR122.

E. coli cells were transformed by standard procedures, while P. aeruginosa cells was transformed by the method of Olsen et al. (J. Bacteriol., 150:60-69 (1982)). P-Gal activity as described above.

The lacZ reporter fusions were constructed in the broad-host range vector pBBR122, which has been reported to replicate in a wide variety of Gram-negative species (MoBitec). To control gene expression, the temperature sensitive C1 repressor polypeptide from the thermoinducible mutant of bacteriophage P1 was used. The lacZ gene was transcriptionally fused to two promoters containing operator sites for C1: Op72 and AP. The nucleic acid encoding a temperature sensitive C1 polypeptide was placed under the transcriptional control of a LacI-regulated promoter, thereby providing regulation of C1 polypeptide expression in strains that express the lacI gene. To enhance binding of the C1 repressor polypeptide to its operator, the bof gene including its own promoter, was cloned 3′ of the c1 gene.

Expression of lacZ was examined in E. coli from two temperature sensitive C1-regulated promoters. In the absence of C1 polypeptide, the promoter strength of AP was similar to the Op72 promoter (Table 5), suggesting the high intrinsic strength of the Op72 promoter even though it does not contain consensus −10/−35 hexamers. When C1 polypeptide was expressed under repressed conditions from the LacI-regulated promoter, β-Gal activity was significantly decreased from both promoters indicating that C1 polypeptide can effectively repress transcription.

TABLE 5
Basal and induced activities from lacZ fusions to the
C1-regulated promoter in E. coli DH5a.

| | | | Miller units | | |
|---|---|---|---|---|---|
| | | | | Induced | |
| | C1 | | Basal activity | activity | Fold- |
| Construct | repressor | IPTG | (31° C.) | (42° C.) | induction |
| Control | + | − | 2.32 (0.4) | 5.34 (0.9) | 2.30 |
| Control | + | + | 2.07 (0.2) | 4.19 (0.3) | 2.02 |
| Op72lacZ | − | − | 930 (11.4) | 759 (1.8) | 0.82 |
| Op72lacZ | − | + | 1199 (64.5) | 890 (6.8) | 0.74 |
| Op72lacZ | + | − | 1.64 (0.3) | 582 (46) | 355 |
| Op72lacZ | + | + | 0.24 (0.1) | 380 (25) | 1583 |
| APlacZ | − | − | 1330 (11.2) | 916 (9.5) | 0.69 |
| APlacZ | − | + | 1339 (81) | 845 (11.2) | 0.63 |
| APlacZ | + | − | 112 (14.4) | 669 (195) | 6 |
| APlacZ | + | + | 25 (0.9) | 450 (6.3) | 18 |
| Overnight cultures were diluted 1:100 and grown to an OD₆₀₀ of 0.1 in LB at 31° C. in the presence or absence of 60 mM IPTG. Cells were collected at 2,500 x g for 10 minutes at room temperature and resuspended in fresh LB. The culture was then divided equally and incubated at 31° C. with additional 60 mM IPTG or at 42° C. for 2 hours prior to assaying | | | | | |
| # for β-Gal activity (OD₆₀₀ of about 0.6). The control vector is identical to the lacZ expression vectors but lacks the C1-regulated promoter. Values are averages of multiple cultures assayed in triplicate (±standard deviation). | | | | | |

The addition of IPTG, which prevents the chromosomally encoded LacI provided by the DH5a cells from binding to the promoter driving C1 polypeptide expression, further reduced basal activity from both promoters under repressed conditions. However, Op72 has a lower basal activity than AP producing about 0.24 as compared to 25 Miller units, respectively (Table 5). This probably reflects the increased ability of the C1 repressor polypeptide to inhibit transcription by binding to the two overlapping C1-operators located within Op72. This level of repression was not detectable above background levels indicating that the repression of Op72 was very efficient which is important when cloning toxic gene products.

To examine the levels of induction from the C1-regulated promoter, the cultures were grown under repressing conditions, divided equally, and shifted to inducing conditions for 2 hours in the absence of IPTG (Table 5). This resulted in induction/repression ratios of up to 1500-fold. Thus, the efficiency of repression can be from 2 to 3 orders of magnitude and is significantly better than the 300-fold induction resuts obtained using either the lambda p_{L}/cI857 thermal induction system (Remaut et al., Gene, 15:81-93 (1981)) or the P_{BAD} promoter in complex medium (Guzman et al., J. Bacteriol., 177:4121-4130 (1995)). The induction/repression ratios for the AP were much lower due to the higher basal activity of this promoter and ranged up to 18-fold.

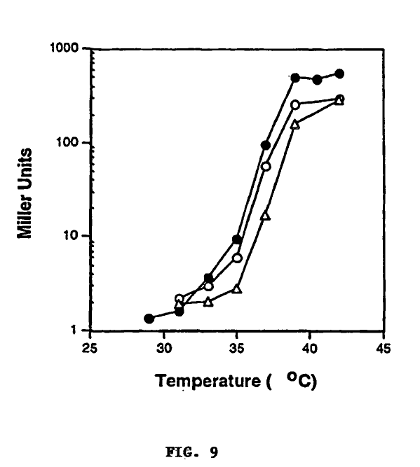

To assess the ability to modulate the temperature sensitive C1-regulated promoters, the extent of induction at different temperatures in three E. coli strains was measured. The results indicated that by varying the temperature, it was possible to modulate induction (FIG. 9). Further, for E. coli DH5a and ER1793 cells, maximal induction was achieved at 39° C., suggesting that it was not necessary to shift the temperature to 42° C. to achieve thermal instability of C1 polypeptides. This may reduce any pleiotropic effects seen at elevated temperatures and is in contrast to the lambda p_{L}/cI857 thermal induction system in which induction at 42° C. is required to inactivate the cI857 repressor. The kinetics of temperature sensitive C1-regulated promoter induction also argue that (1) the temperature sensitive C1-regulated promoters have a fast rate of induction and (2) incubation under inducing conditions need only be maintained for 60 minutes to achieve near maximal induction.

In contrast to the results obtained from E. coli, when c1 was expressed in cis, the basal activity of both promoters was similar and only 2- to 3-fold above background levels were observed (Table 6). This suggested that both promoters were being effectively repressed by C1 polypeptide in P. aeruginosa and that the dual C1 operator sites of Op72 was only marginally more effective than the single operator site of AP. Further, when the cultures were placed under inducing conditions, derepression from both promoters was modest (e.g., up to 4-fold). Levels of induction were not improved when a weaker promoter driving c1 was utilized. This is in stark contrast to E. coli and suggests factors specific to E. coli, but lacking in P. aeruginosa, are needed to facilitate C1 thermal instability.

TABLE 6
Basal and induced activities from lacZ fusions to the C1-regulated
promoter in P. aeruginosa.

| | | Miller units | | |
|---|---|---|---|---|
| | | Basal activity | Induced activity | Fold- |
| Construct | C1 repressor | (31° C.) | (42° C.) | induction |
| Control | + | 32.5 (2.6) | 25.6 (4.5) | 0.8 |
| Op72lacZ | − | 11348 (1410) | 13472 (1773) | 1.2 |
| Op72lacZ | + | 67 (5.9) | 82.1 (14.9) | 1.2 |
| APlacZ | − | 19791 (2782) | 17113 (720) | 0.9 |
| APlacZ | + | 84.6 (7.6) | 338.2 (68.1) | 4.0 |
| Overnight cultures carrying the reporter constructs were diluted 1:100 and grown to an OD₆₀₀ of 0.1 in LB at 31° C. Cells were collected at 2,500 x g for 10 minutes at room temperature and resuspended in fresh LB. The culture was then divided equally and incubated at 42° C. or 31° C. for 3 hours prior to assaying for β-Gal activity (OD₆₀₀ at time | | | | |
| #of harvesting was about 0.6). The control vector is identical to the lacZ expression vectors but lacks the C1-regulated promoter. Values are averages of multiple cultures assayed in triplicate (±standard deviation). | | | | |

To increase the levels of derepression at elevated temperatures, the amount of C1 polypeptide was modulated at the level of mRNA expression. The E. coli lacI gene was transcriptionally coupled to the lacZ gene so that expression of both genes were controlled from the C1-regulated promoter. As the promoter driving c1 expression contains a LacI operator site, the level of c1 expressed can be modulated by the addition of IPTG. At low temperature and in the absence of IPTG, this resulted in a dramatic increase in β-Gal expression from both promoters (Table 7) to levels obtained when the constructs lack c1 (Table 6). Thus, under these conditions, LacI is being expressed sufficiently to switch off C1 expression effectively, resulting in both LacZ and LacI expression. Exposure to IPTG, which binds LacI thereby preventing it from binding to the promoter driving c1, resulted in a 55-fold decrease in β-Gal activity to levels about 3-times the background activity. Therefore, at low temperature and in the presence of IPTG, it is possible to repress LacZ expression effectively using a combination of C1 and LacI polypeptides.

TABLE 7
Basal and induced activities from lacZ-lacI fusions
to the Op72 promoter in P. aeruginosa.

| | | Miller units | | |
|---|---|---|---|---|
| | IPTG | Basal activity | Induced activity | Fold- |
| Construct | (mM) | (31° C.) | (42° C.) | induction |
| Control | 2 | 95.9 (8.3) | 121.1 (6.9) | 1.3 |
| Op72lacZLacI | 0 | 17625 (1516) | 23191 (489) | 1.3 |
| Op72lacZLacI | 2 | 317.4 (45.4) | 403.4 (19.6) | 1.3 |
| Op72lacZLacI | 0.2 | 320.9 (40.2) | 16682 (1847) | 52 |
| Op72lacZLacI | 0.06 | 339.8 (26.1) | 20106 (666) | 59 |
| Op72lacZLacI | 0.02 | 1401 (212) | 22583 (2775) | 16 |
| Overnight cultures were diluted 1:100 and grown to an OD₆₀₀ of 0.1 in LB at 31° C. in the presence or absence of IPTG as indicated. Cells were collected at 2,500 x g for 10 minutes at room temperature and resuspended in fresh LB medium. The culture was then divided equally and incubated at 31° C. with additional IPTG or at 42° C. for 3 hours to | | | | |
| #titrate out the IPTG (OD₆₀₀ at time of harvesting was about 0.6) prior to assaying for β-Gal activity. The control vector is identical to the lacZ expression vector but lacks the Op72 promoter. Values reported are averages of multiple cultures assayed in triplicate (±standard deviation). | | | | |

To investigate levels of derepression, the cultures were incubated under repressed conditions in the presence of IPTG, divided equally in fresh medium lacking IPTG, and incubated at the elevated temperature for 3 hours. Depending on the concentration of IPTG, maximal derepression of the promoter can be achieved (Table 7). This level of derepression (59-fold induction) cannot be obtained after 3 hours by titration of the IPTG alone illustrating that both the temperature switch and titration of the IPTG was required.

The temperature sensitive C1-regulated promoter system provided herein displayed extremely tight repression, modulation of expression, and up to 1500-fold increase in β-Gal activity after 2 hours post induction in E. coli. Further, the high strength of Op72 suggests that it may also be suitable for the overexpression of genes. The temperature sensitive C1-regulated promoter system effectively repressed transcription in P. aeruginosa, but exhibited only modest induction. A two component regulatory system was developed combining C1 with LacI, which resulted in a 59-fold induction in gene expression. The promoters provided herein can be used to control gene expression in Gram-negative bacteria.

### Example 8

### A P1 Phagemid for Delivery to Gram-Negative Bacteria

Only a limited number of bacteria (e.g., Haemophilus influenzae, Streptococcus pneumoniae, and Bacillus subtilis) can be transformed by natural competence (Lorenz and Wackemagel, Microbiol. Rev., 58:563-602 (1994). A number of factors, however, such as prolonged incubation with CaCl₂, treatment of bacteria with dimethyl sulfoxide, hexaminecobalt, and dithiothreitol in the presence of cations, or addition of polyethylene glycol can induce artificial competence (Hanahan et al., Methods Enzymol., 204:63-113 (1991)). Genetic information, for example, can be delivered to E. coli K12 by transformation of chemically- or electro-competent cells, phage transduction, and conjugational mating (Benedik, Mol. Gen. Genet., 218:353-354 (1989); Dower et al., Nucleic Acids Res., 16:6127-6145 (1988); and Hanahan et al., Methods Enzymol., 204:63-113 (1991)). However, many bacterial species of clinical, environmental, and industrial importance cannot be made competent.

Recombinant DNA manipulations in bacteria typically involve initial cloning and molecular analyses in E. coli, followed by reintroduction of the cloned DNA into the original host genetic background for studies of virulence gene expression and reverse genetics. Some species are recalcitrant to standard transformation techniques. Therefore, genetic analysis of these species is largely impaired. In addition, most bacterial species possess restriction/modification systems that have evolved to protect the cell from foreign DNA (Bickle and Krüger, Microbiol Rev., 57:434-450 (1993)). Modification of DNA can differ between species and among strains of the same species, raising additional barriers to gene transfer. To facilitate the movement of DNA, some transformation protocols are limited to specific strains that are defective in one or more restriction systems (Novick, The staphylococcus as a molecular genetic system. In Molecular Biology of the Staphylococci, pp. 1-37. Edited by R. P. Novick. NY: VCH Publishers (1990) and Takagi and Kisumi, J. Bacteriol., 161:1-6 (1985)). Non-specific barriers such as high intra- or extra-cellular nuclease activity can also have profound effects on transformation efficiency (Omenn and Friedman, J. Bacteriol., 101:921-924 (1970); Shireen et al., Can. J. Microbiol., 36:348-351 (1990); and Wu et al., Appl. Environ. Microbiol., 67:82-88 (2001)). Genetic exchange between mutated laboratory strains and clinical or environmental isolates can be hampered by the lack of alternative methods for the delivery of genes.

The ability to electroporate protoplasts, spheroplasts, and intact cells has advanced microbiological studies in organisms where other transformation procedures have failed (Chassy et al., Tibtech 6:303-309 (1988)). However, the generation of cells lacking cell walls can be difficult. In addition, these methods normally require optimization of numerous strain-dependent parameters for efficient transformation and regeneration. Transformation efficiencies of intact cells can be highly variable depending on the growth media, growth phase, and final concentration of cells, composition of the electroporation medium, electric parameters, and conditions used to select for transformants.

In the following example, the construction of a phagemid vector, P1pBBR122-T, which can be used for cloning in E. coli or several Gram-negative hosts is provided. In addition, the development of a P1 phage delivery system that has great use for the movement of P1pBBR122-T between a variety of clinically relevant Gram-negative species is described.

The bacterial strains, plasmids, and phage used in this example are listed in Table 8. Bacterial cells were grown in Luria-Bertani medium (LB), LC medium (LB containing 10 mM MgSO₄ and 5 mM CaCl₂) or LB agar. Selection for plasmids was accomplished by the addition of kanamycin (Kan 50 μg mL⁻¹), ampicillin (Amp 100 μg mL⁻¹) or carbenicillin (500 μg mL⁻¹) as needed. DNA manipulations were carried out by standard methods.

TABLE 8
Characteristics, and origins of bacteria, plasmids and
phage used in this example

| Bacteria, plasmid or | | Source or |
|---|---|---|
| phage | Description or Genotype | Reference^{‡} |
| Bacteria | | |
| E. coli | | |
| P1 lysogen | C600 (P1Cm clts.100) | Rosner |
| C600 | recA+ | Promega |
| JM101 | recA+ | NEB |
| DH5a | recA− | Gibco BRL |
| JM109 | recA− | NEB |
| EC-1 | Urine clinical isolate, | MUSC |
| | Ampicillin resistant | |
| EC-2 | Urine clinical isolate, | MUSC |
| | Ampicillin sensitive | |
| P. aeruginosa | | |
| PAO1 | Clinical isolate | PGSC |
| PA-1 | Clinical isolate | MUSC |
| S. flexneri | Serotype 2b | ATCC 12022 |
| S. dysenteriae | 60R | Dr. Butterton^{†} |
| C. freundii | Produces restriction endonuclease | ATCC 8090 |
| | CfrA1 | |
| K. pneumoniae | Wild-type | ATCC 10031 |
| Plasmids | | |
| pBluescript II SK⁺ | Cloning vector, ColE1 origin | Stratagene |
| pBBR122 | Cloning vector, Broad host | MoBiTec |
| | range origin | |
| Phage | | |
| P1Cm clts.100 | Thermoinducible P1Cm | Rosner |

^{‡}Rosner, Virology 49: 679-689 (1972); NEB, New England Biolabs, Ltd; MUSC, Medical University of South Carolina, Department of Pathology and Laboratory Medicine; PGSC, Pseudomonas Genetic Stock Center, East Carolina University; ATCC, American Type Culture Collection; Cm, chloramphenicol marker.
^{†}Dr. Joan Butterton, Massachusetts General Hospital, Boston.

P1pBBR122-T was constructed as described in Example 2, and thermal induction of P1Cm c1ts100 lysogens harboring the plasmid P1pBBR122-T was performed as described in Example 4. In addition, the phagmid delivery and analysis were performed as set forth in Example 5.

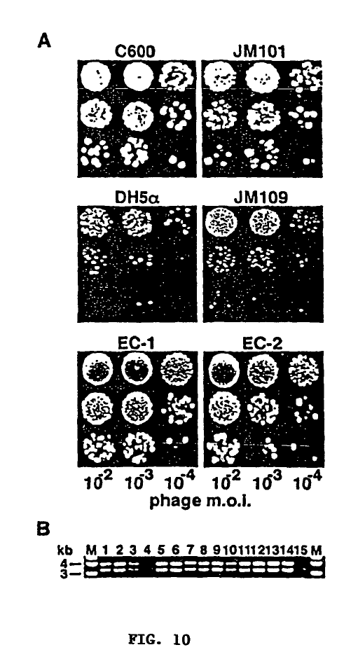

The ability to deliver the phagemid to multiple strains of bacteria was tested with laboratory strains and clinical isolates of E. coli. Since the wild-type RecA polypeptide is thought to be necessary for stable transduction (Sandri and Berger, Virology 106:14-29 (1980), recombination-competent (C600 and JM101) and recombination-deficient strains (DH5a and JM109) were tested. Increasing titers of phage were added to fixed numbers of bacterial cells and limited to a single round of infection by the addition of 10 mM sodium citrate. After infection, phagemid-containing transductants were selected by virtue of their ability to grow in the presence of antibiotics. The total number of transductants increased progressively as the moi increased (FIG. 10A). Antibiotic-resistant colonies were not recovered when the phage lysate or cells were tested alone.

Successful delivery of P1pBBR122-T was confirmed by extraction of this plasmid from representative isolates. Antibiotic-resistant transductants harbored plasmid DNA whose migration was identical to that originally seen in the parent strain (FIG. 10B). Restriction enzyme digestion demonstrated that gross deletions or genetic rearrangements in P1pBBR122-T did not occur as a consequence of packaging or recircularization. Acquisition of P1pBBR122-T did not result in displacement (incompatibility) of native plasmids in clinical isolates.

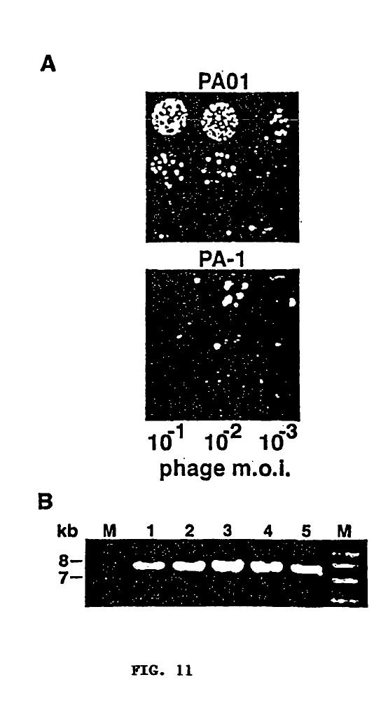

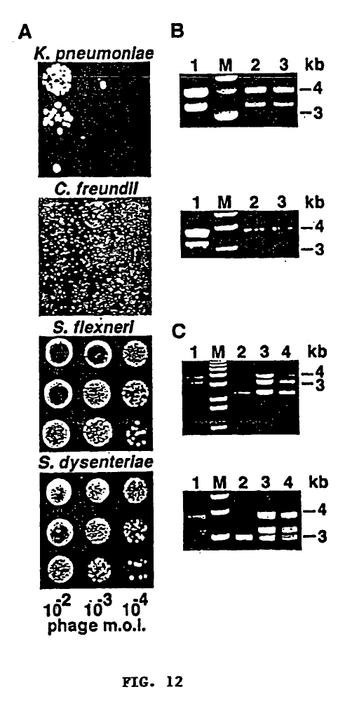

Transduction of the phagemid was tested in various Gram-negative bacteria including P. aeruginosa, K pneumoniae, C. freundii, S. flexneri, and S. dysenteriae. All bacteria were successfully transduced by the P1 delivery system (FIGS. 11A and 12A). The P. aeruginosa clinical isolate PA-I was transduced at a lower efficiency than the laboratory strain PAO1 (FIG. 11A). It is noteworthy that a similar effect has been reported for electroporation of P. aeruginosa isolates from lung sputum of cystic fibrosis patients and wild-type strains isolated from different sources for other Gram-negative species (Diver et al., Anal. Biochem., 189:75-79 (1990) and Wirth et al., Mol. Gen. Genet., 216:175-177 (1989)). Functionality of the pBBR122 origin of replication among the Gram-negative species was confirmed by extraction and analysis of P1pBBR122-T from representative transductants (FIGS. 11A, 12B, and 12C).

The majority of bacteria carry plasmids or lysogenized phage that protect their host by expressing potent activities that prevent infection by other phages (Dinsmore and Klaenhammer, Mol. Biotechnol., 4:297-314 (1995) and Synder, Mol. Microbiol., 15:415-420 (1995). This is particularly relevant for transduction of environmental P. aeruginosa strains since 40 percent of isolates recovered from natural ecosystems (lake water, sediment, soil, and sewage) contain DNA sequences homologous to phage genomes (Ogunseitan et al., Appl. Environ. Microbiol., 58:2046-2052 (1992)). The P1 delivery system, however, does not appear to be under the constraints of superinfection exclusion since P1pBBR122-T can be successfully delivered to a P1 lysogen. The phagemid was also introduced by infection into S. flexneri and S. dysenteriae strains harboring a natural resident plasmid (FIG. 12C).

Since the various Gram-negative bacteria accepted DNA packaged from another bacterial genus (E. coli), this suggested protection of the DNA by the P1 Dar proteins, lack of a restriction endonuclease recognition sequence in the transduced plasmid DNA, or the species tested did not possess an effective restriction/modification system. The results obtained with the different bacteria indicate that P1 phage can be used to transform many different Gram-negative bacteria.

In this example, phagemid DNA was readily introduced into a variety of Gram-negative bacteria including E. coli via P1 phage. Phagemid P1pBBR122-T is a relatively small plasmid (7.3 kb) containing one or two antibiotic-resistance determinants (Kan^{R} and/or Amp^{R}). Both are readily selectable and/or scoreable markers for Gram-negative bacteria. The ability to screen presumptive transductants for antibiotic-resistance was a reliable and simple means of phenotypically confirming transduction of the phagemid to E. coli and other Gram-negative bacteria. The ability of the pBBR122 origin of replication to function in various Gram-negative bacteria was demonstrated herein. Thus, these results demonstrate that the P1 phage delivery methods and materials provided herein can be used in various bacteria including Yersina pestis, Yersina pseudotuberculosis, and Salmonella typhimurium.

### Example 9

### Doc-Mediated Cell Killing in S. flexneri Using Vectors Containing a C1-Regulated Promoter

Shigella species are capable of causing acute, debilitating diarrheal disease in humans. While S. dysenteriae causes the most severe diarrheal illness reflected in high mortality rates, S. flexneri remains the leading cause of shigellosis in most of the developing world (Keusch et al., J. Pediatr. Infect. Dis., 8:713-719 (1989) and Navia et al., J. Clin. Microbiol., 37:3113-3117 (1999). Bacteriophage P1 lysogenizes E. coli in a stable fashion, in part, due to the plasmid addiction system that kills plasmid-free segregants via a toxin known as Doc (death on curing; Lehnherr et al., J. Mol. Biol. 233:414-428 (1993)). In E. coli, Doc-mediated post-segregational killing requires the antitoxin/toxin system, mazEF (Hazan et al., J. Bacteriol., 183:2046-2050 (2001)). As mazEF is chromosomally encoded and activated by starvation conditions, it has been suggested that this system may play a role in programmed cell death (Aizenman et al., PNAS, 93:6059-6063 (1996)). In silico analysis has identified orthologous systems in both Gram-negative and -positive species suggesting that mazEF may be conserved among prokaryotes (Engelberg-Kulka et al., ASM News, 67:617-624 (2001) and Mittenhuber, J. Mol. Microbiol. Biotechnol. 1:295-302 (1999)). In one embodiment, the development of a regulated promoter system that exhibits a similar range of regulation, and a high level of stringency irrespective of its use in either E. coli or S. flexneri is described.

To control gene expression, the lacZ reporter sequence was placed under the control of a promoter regulated by the temperature sensitive C1 repressor polypeptide from the broad-host-range bacteriophage P1. Nucleic acid encoding the temperature sensitive C1 repressor polypeptide was placed under the transcriptional control of LacI, thereby providing a dual means of regulation by varying both the temperature and concentration of IPTG. Using the C1/LacI regulated promoter system to control expression of the bacteriophage P1 post-segregational killer protein Doc, the bactericidal effect of Doc was demonstrated in S. flexneri.

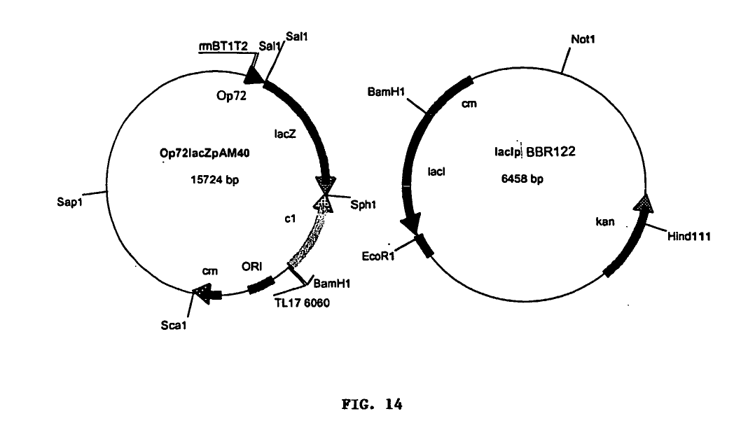

Reporter plasmids were constructed in the Gram-positive/Gram-negative shuttle vector, pAM401 (Wirth and Clewell, J. Bacteriol., 165:831-836 (1986); FIG. 14). The reporter system was placed under the transcriptional control of the C1-regulated promoter Op72. To control gene expression, the temperature sensitive C1 polypeptide from bacteriophage P1 was used. This promoter system functions well in E. coli but to a lesser extent in S. flexneri, primarily due to the inability to achieve derepression at elevated temperatures. To circumvent this, nucleic acid encoding the temperature sensitive C1 repressor polypeptide was placed under the transcriptional control of a LacI-regulated promoter, thereby providing a dual means of regulation in species that express LacI. As S. flexneri lacks a functional lacI homolog, a lacI expression plasmid was constructed (lacIpBBR122; FIG. 14) and where indicated, was co-transformed (Lederberg and Cohen, J. Bacteriol., 119:1072-1074 (1974)) with the lacZ reporter plasmid into S. flexneri. At low temperatures and in the presence of IPTG, C1 polypeptide is expressed and is thermally stable which in turn switches off the expression of the reporter, lacZ. At elevated temperatures and in the absence of IPTG, C1 polypeptide is switched off and is thermally unstable which results in LacZ expression.

To demonstrate the functionality of the dual promoter construct, the activity of the polypeptide produced by the lacZ gene (β-Gal activity) was measured in E. coli DH5α (lacI) and XL1-Blue (lacI^{q}), that express and over-expresses LacI, respectively. Since the promoter driving c1 contained consensus −35/−10 hexamers (TTGACA, SEQ ID NO:47; and TATAAT, SEQ ID NO:48), it was expected that the construct would produce an excess of C1 polypeptide resulting in the efficient repression of the C1-regulated promoter but might only result in the partial derepression at elevated temperatures. In support of this hypothesis basal expression in DH5α was below the limits of detection of the assay, and upon induction at elevated temperature, only a modest level of induction was observed (Table 9). In contrast, basal expression in XL1-Blue cells was extremely high suggesting that the expression of the chromosomally encoded and over-expressed LacI was effectively switching off c1 expression. Upon addition of IPTG, a dramatic decrease in β-Gal expression was observed at levels nearly undetectable by the assay. Furthermore, following exposure to IPTG at low temperature, high levels of induced expression were achieved after only 100 minutes of induction. Therefore, the results indicate that it was possible to achieve low levels of basal expression, and high-induced activity using a combination of C1 polypeptide to control lacZ expression, and LacI polypeptide to control levels of C1 polypeptide produced.

TABLE 9
Basal and induced activities of lacZ fusions to the C1-regulated
promoter in E. coli strains DH5α and XL1-Blue.

| | | | Activity (Miller units) | |
|---|---|---|---|---|
| | Strain | | Basal | Induced |
| Construct | (lacI status) | IPTG (mM) | (31° C.) | (42° C.) |
| Control | DH5α (lacI) | 0 | <0.5 | <0.5 |
| Op72lacZ | DH5α (lacI) | 0 | <0.5 | 11(0.3) |
| Control | XL1-Blue (lacI^{q}) | 0 | <0.5 | <0.5 |
| Op72lacZ | XL1-Blue (lacI^{q}) | 0 | 471(24) | 1578(26) |
| Op72lacZ | XL1-Blue (lacI^{q}) | 2 | <0.5 | 0.5(0.1) |
| Op72lacZ | XL1-Blue (lacI^{q}) | 0.2 | <0.5 | 84(17) |
| Op72lacZ | XL1-Blue (lacI^{q}) | 0.06 | <0.5 | 617(47) |
| Overnight cultures grown at 31° C. at the stated concentration of IPTG were diluted 1:100 and grown to an OD₆₀₀ of about 0.15 in LB under the same conditions. Cells were collected at 2,500 x g for 10 minutes at room temperature and resuspended in fresh LB. Cultures were divided equally and incubated at 31° C. with IPTG at the same concentration | | | | |
| #or at 42° C. without IPTG for 100 minutes (OD₆₀₀ about 0.6). The control strain carried a plasmid containing a promoterless lacZ gene. Miller units are averages of results for multiple cultures (n = 3) followed by the standard deviation in parentheses. | | | | |

<0.5 indicates below the limits of detection for the assay.

The functionality of the dual expression system was tested in S. flexneri ATCC 12022. As S. flexneri does not contain a functional homolog of LacI, it was supplied in trans from a lacI expression plasmid (lacIpBBR122; FIG. 14). Since an insufficient intracellular concentration of LacI would have little effect on C1 polypeptide expression, and an intracellular excess of LacI might generate leakiness from the C1-regulated promoter, a number of different lacI expression plasmids were constructed and evaluated in order to find the optimal concentration of LacI for control of the desired transcriptional elements. In the absence of LacI at low temperatures, β-Gal activity in S. flexneri was below the limits of detection with only modest induction observed at the elevated inducing temperature (Table 10). Co-transformation of both the lacZ and lacI expression plasmids, however, resulted in a dramatic increase in basal expression that could be regulated to concentrations below detectable limits by the addition of IPTG. Furthermore, high levels of induced expression were achieved by the elevation of temperature and the titration of IPTG (Table 10). This level of induced expression was significantly higher using the dual C1/LacI regulated promoter construct as compared to the system regulated by C1 alone. Because the basal expression levels was below the limit of detection of the standard colorimetric assay for β-Gal, the activity of the enzyme was also measured using a chemiluminescent substrate in order to determine the level of expression from the regulated genetic elements (Table 10). The activity observed ranged from 2.1×10⁴ units during basal conditions to 8.1×10⁷ units under induced conditions. This represented an approximate 3700-fold range of regulation. These results are similar to, if not better than, the results obtained using regulated promoter systems described for E. coli (Guzman et al., J. Bacteriol. 177:4121-4130 (1995) and Lutz and Bujard, Nucleic Acids Res., 25:1203-1210 (1997)).

TABLE 10
Basal and induced activities of lacZ fusions to the C1-regulated promoter in S. flexneri.

| | | | Activity | | | |
|---|---|---|---|---|---|---|
| | LacI | | Basal (31° C.) | | Induced (42° C.) | |
| Construct | repressor | IPTG (mM) | Miller units | R.L.U. | Miller units | R.L.U. |
| Control | − | 0 | <0.5 | nd | <0.5 | nd |
| Op72lacZ | − | 0 | <0.5 | nd | 18(0.3) | nd |
| Op72lacZ^{a} | + | 0 | 324(30) | 7.7 × 10⁷ | 392(44) | 8.7 × 10⁷ |
| Op72lacZ^{a} | + | 1 | <0.5 | 9.8 × 10^{3b} | 283(4) | 7.9 × 10⁷ |
| Op72lacZ^{a} | + | 0.2 | <0.5 | 2.1 × 10⁴ | 317(24) | 8.1 × 10⁷ |
| Op72lacZ^{a} | + | 0.06 | 0.8(0.3) | 3.1 × 10⁵ | 303(6) | 7.4 × 10⁷ |
| Overnight cultures grown at 31° C. at the stated concentration of IPTG were diluted 1:100 and grown to an OD₆₀₀ of about 0.1 in LB under the same conditions. Cells were collected at 2,500 x g for 10 minutes at room temperature and resuspended in fresh LB. Cultures were then divided equally and incubated at 31° C. with IPTG at the same concentration or at 42° C. without IPTG for 80 minutes (OD₆₀₀ about 0.6). | | | | | | |
| # The control strain carried a plasmid containing a promoterless lacZ gene. Miller units (10) are averages of results for multiple cultures (n = 3) followed by the standard deviation in parentheses. Where indicated, lysates were also measured using the galacto-star chemiluminescent reporter gene assay (Applied Biosystems) and are presented as relative light units (R.L.U)/OD₆₀₀ of culture. | | | | | | |

<0.5 indicates below the limits of detection for the assay.
^{a}denotes S. flexneri co-transformed with the lacI expression plasmid.
^{b}denotes below the linear range of the luminometer.
nd, not determined.

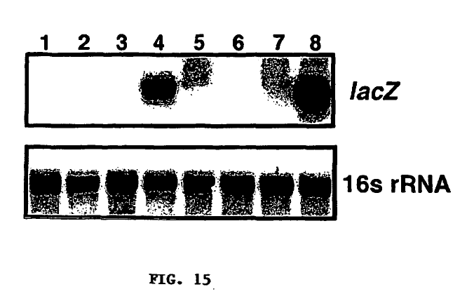

To analyze the regulation of lacZ expression at the transcriptional level, northern blot analysis was performed. RNA was prepared from cultures carrying promoterless lacZ constructs and from cultures carrying the reporter plasmids under repressed and derepressed conditions. Transcripts were not detected from control cultures or from cultures prepared under repressed conditions using lacZ (SalI/SphI generated fragment) as a probe for either S. flexneri or E. coli (FIG. 15, lanes 1, 2, 3, 5, 6 and 7). In contrast, under induced conditions, transcripts were detected from both S. flexneri and E. coli harboring the reporter constructs (FIG. 15, lanes 4 and 8). Thus, northern analysis confirmed that the regulation of lacZ expression occurs primarily at the transcriptional level and suggests that the promoter system is tightly repressed.

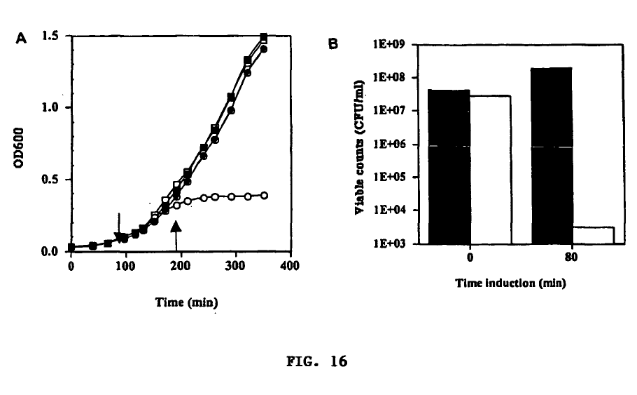

To assess the controlled killing of bacteria via Doc, nucleic acid encoding a Doc polypeptide was placed under the control of the C1-regulated promoter. No difference in the growth of the cultures harboring the doc expression plasmid was observed upon induction using temperature shift alone. However, when the same cultures carrying the doc expression plasmid were co-transformed with the lad expression plasmid, induction using a temperature shift in the absence of IPTG resulted in growth arrest (FIG. 16A). This indicated that LacI was required to switch off c1 expression in order to achieve sufficient levels of Doc. Interestingly, expression of the E. coli toxic protein Gef (Poulsen et al., Mol. Microbiol., 3:1463-1472 (1989)) did not mediate growth inhibition under the same conditions.

To investigate whether Doc exerts a bacteristatic or bactericidal effect in S. flexneri, cultures where plated out immediately prior to induction and after 80 minutes induction, and were allowed to recover overnight under repressed conditions (31° C., 1 mM IPTG). This resulted in a 10⁴ reduction in the number of colony forming units (FIG. 16B). A reduction in colony forming units was not observed for the control cultures. These results suggest that Doc exerts a bactericidal effect in S. flexneri. Although the target of Doc is unknown, as P1 can lysogenize a wide variety of Gram-negative species, it is not unreasonable to speculate that the target of Doc may be conserved. In silico analysis has identified mazEF orthologs in both Gram-negative and -positive bacteria (Mittenhuber, J. Mol. Microbiol. Biotechnol., 1:295-302G (1999)) leading to the possibility that Doc-mediated cell death by mazEF may also occur in species other than E. coli.

### Example 10

### Thermally Regulated Broad-Spectrum Promoter System for Use in Gram-Positive Species

In this exmple, the ability of promoters regulated by temperature sensitive C1 polypeptides to function in Enterococcus faecium, Enterococcus faecalis, and Staphylococcus aureus was evaluated. Breifly, using the lacZ gene to monitor gene expression, the strength, basal expression, and induced expression of synthetic promoters carrying C1 operator sites were examined. The promoters exhibited extremely low basal expression and, under inducing conditions, gave high levels of expression (100 to 1000-fold induction). The promoter system was modulated by temperature and showed rapid induction. In addidion, the mechanism of regulation occurred at the level of transcription. Controlled expression with the same constructs was also demonstrated in the Gram-negative bacterium Escherichia coli. However, low basal expression and the ability to achieve derepression was dependent on both the number of mismatches in the C1 operator sites and the promoter driving C1 polypeptide expression. Since the promoters were designed to contain conserved Gram-positive promoter elements and were constructed in a broad-host-range plasmid, this system provides a new opportunity for controlled gene expression in a variety of Gram-positive bacteria.

E. coli DH5α (Gibco-BRL), S. aureus RN4220 (kindly provided by Jean Lee, Channing Laboratory, Boston), E. faecalis ATCC 47077, and E. faecium ATCC 12952 were used. The growth media used for each bacterial strain were as follows: Luria Bertani broth for E. coli; tryptic soy broth for S. aureus; brain heart infusion broth for E. faecalis, and Todd Hewitt broth for E. faecium.

The reporter plasmids were constructed in the Gram-negative/Gram-positive shuttle vector pAM401 (Wirth et al., J. Bacteriol., 165:831-836 (1986)). The lacZ gene was amplified by PCR using pBBR122lacZ as template with the upstream primer 5′-AGGACGGTCGACTAAGGAGGTGAAAAGTATGGTCGTTTTACAAGCTCG (SEQ ID NO:49) and downstream primer 5′-TCCTCCGCATGCTCCCCCCTGCCCGGTTAT (SEQ ID NO:50), which contained SalI and SphI restriction sites (underlined) for cloning into the SalI and SphI sites of pAM401. The upstream primer also contained a RBS (5′-TAAGGAGG, SEQ ID NO:51) positioned 8 bp upstream of a start codon (bold) to initiate translation.

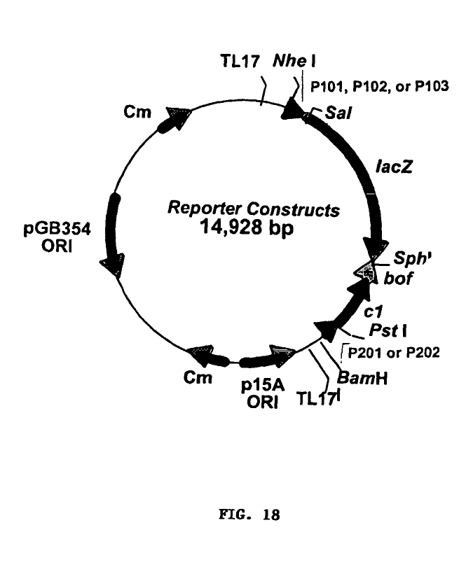

The C1-regulated promoters (FIG. 17) were obtained by annealing complementary oligonucleotides that contained partial and full SalI overhangs (5′ and 3′ ends, respectively). The promoters were cloned (in the same orientation as lacZ) into the SalI site of pAM401, thereby recreating the 3′ SalI site only. To increase the number of cloning sites, the oligonucleotides also contained a SpeI site at the 5′ end. To stop readthrough from cryptic promoters into the 5′ end of the expression cassette, the transcriptional terminators TL₁₇ were cloned into the SpeI site. To prevent ‘runaway’ transcription, the terminators were also cloned at the 3′ end of the expression cassette (EcoRV site). To control gene expression, the coding sequences for the C1 polypeptide and Bof modulator polypeptide were inserted initially into the cloning vector pBluescript II SK⁺ (Stratagene). The forward PCR primers used to amplify, c1 and bof sequences contained both an RBS and restriction endonuclease site. To incorporate both of these features, c1 and bof sequences were amplified by PCR using a semi-nested PCR strategy. c1 was amplified using the thermosensitive mutant of P1 as template with the forward primer 5′-TAAGGAGGTGAAAAGTATGATAAATTATGTCTACGGC (SEQ ID NO:52) and reverse primer 5′-CTAGCTGAATTCCTATTGCGCGCTTTCGGGGTTG (SEQ ID NO:53). After 10 amplification cycles, an aliquot (1 μL) was then reamplified with the forward nested primer 5′-CGCAGTGAATTCTAAGGAGGTGAAAAGTATG (SEQ ID NO:54) and the same reverse primer. The forward primers contained an RBS upstream of the start codon (bold), and both primers contained EcoRI restriction sites (underlined sequence) for cloning into the corresponding sites of pBluescript II SK⁺. Similarly, the forward primer 5′-TAAGGAGGTGAAAAGTATGAAAAAGCGATACTACACAG (SEQ ID NO:55), reverse primer 5′-GTAGTAGCATGCGGTGAGCAAACAGCCAT (SEQ ID NO:56), and nested forward primer 5′-GCTAGGAAGCTTTAAGGAGGTGAAAAGTATG (SEQ ID NO:57) were used to amplify bof sequences using bacteriophage P1 DNA as template. The bof primers contained HindIII and SphI sites (underlined). However, bof was cloned 3′ of c1 into the HindIII and HindII sites of pBluescript II SK⁺. To drive expression of c1 and bof, complementary oligonucleotides containing promoter elements (FIG. 17) were cloned upstream of c1/bof into the BamHI/PstI sites of pBluescript II SK⁺. The ‘promoter-c1.bof fragments’ with BamHI/SphI overhangs were then cloned into the corresponding sites of pAM401 lacZ to create the final reporter constructs (FIG. 18).

E. coli was transformed according to standard procedures. E. faecalis and E. faecium were electroporated according to Friesenegger et al. (FEMS Microbiol. Lett., 79:323-328 (1991)) except cells were resuspended at one-hundredth of their original culture volume. S. aureus was electroporated by the method described by Lee (1995 Electroporation protocols for Microorganisms, p. 209-215. In J. A. Nickoloff (ed.), Methods in Molecular Biology, vol. 47. Humana Press Inc., Totowa, N.J.). Chloramphenicol was used to select for plasmids at the following concentrations: 25 μg/mL, E. coli; 20 μg/mL, E. faecalis; 5 μg/mL, E. faecium; and 15 μg/mL, S. aureus.

RNA was extracted from E. faecium, E. faecalis, and S. aureus using Qiagens RNeasy kit according to the manufacturers' instructions with the following modification. To break open the bacterial cells, the samples were vortexed continuously for 10 minutes in the presence of acid washed glass beads (212-300 μM). RNA (up to 10 μg) was vacuum blotted onto Duralon UV membrane (Stratagene) using a slot blot apparatus. Two identical RNA blots were prepared for each species. Both membranes were probed with a ³⁵S-tailed (Roche) oligonucleotide complementary to either lacZ (5′-CGCTCAGGTCAAATTCAGACGGCAAACGA, SEQ ID NO:58) or a conserved region of 16s rRNA (5′-CCAACATCTCACGACACGAGCTGACGACAA, SEQ ID NO:59). Hybridization was performed in 1× Denhardts' solution, 4×SSC, 50 μg/mL poly(A), 500 μg/mL salmon sperm, 10% dextran sulphate, and 45% formamide at 37° C. Washing was performed at 37° C. at a final stringency of 0.5×SSC and 0.1% SDS. The membranes were visualized using a phosphorimager.

β-Gal activity was assayed according to Miller (Experiments in Molecular Genetics. Cold Spring Harbor Laboratory Press, Cold Spring Harbor, N.Y. (1972)) except that the cells were permeabolized with four drops of chloroform and two drops of 0.1% SDS.

Compilation analysis of Gram-positive promoters (Graves and Rabinowitz, J. Biol. Chem., 261:11409-11415 (1986) and Helman, Nucleic Acids Res. 23:2351-2360 (1995)) was performed, and three promoters (P101, P102, and P103; FIG. 17) were designed containing conserved Gram-positive elements. The conserved elements consisted of the −35/−10 hexamers, an ‘A’ tract, a single ‘T’ 5′ of the −35 hexamer, a ‘TG’ dinucleotide 5′ of the −10 hexamer, and two ‘A’ nucleotides 3′ of the −10 hexamer (FIG. 17). The three promoters differed by a single nucleotide within the −10 hexamer (P101 to P102) or by the addition of ‘TG’ nucleotides (P102 to P103; FIG. 17). The promoters also were designed to contain two, partially overlapping C1 operator sites. Placement of the C1 operators downstream of the −10 hexamer resulted in only partial repression in the presence of C1 polypeptide in E. coli. Consequently, the operator on the top strand was placed between the −35/−10 hexamers, while the operator on the bottom strand completely covered the −10 hexamer. In the presence of C1 polypeptide, this placement was expected to more effectively prevent transcription by occlusion of the RNA polymerase and/or masking of the promoter elements. However, as a result of having optimized promoter elements, P103 carried five mismatches, and P102 carried one mismatch to the consensus C1 operator sequence. Nevertheless, these operator sites were expected to be effective since functional C1 binding sites containing mismatches to the consensus sequence have been identified throughout the P1 plasmid (Citron et al., J. Biol. Chem., 264:3611-3617 (1989)).

The promoters were transcriptionally fused to the lacZ reporter gene to monitor gene expression. To control expression, the temperature sensitive C1 repressor polypeptide was used. The amount of C1 polypeptide produced is related to the effectiveness of a promoter system. Low amounts of C1 polypeptide can result in partial repression, while too much C1 polypeptide can result in the inability to achieve derepression. Thus, the c1 gene was placed under the transcriptional control of one of two designed promoters (P201 or P202; FIG. 17), each of which has consensus −35/−10 hexamers, but differ in their spacer sequence. Variations in the spacer sequence can alter promoter strength by up to 400-fold (Jensen and Hammer, Appl. Environ. Microbiol., 64:82-87 (1998)). The sequence of spacers between the consensus sequences modulates the strength of prokaryotic promoters.

To enhance binding of the C1 repressor polypeptide to its operator, the bof gene was cloned 3′ of the c1 gene. To ensure efficient translation, the primers amplifying lacZ incorporated a contrived Gram-positive RBS (TAAGGAGG(N)₈ATG; SEQ ID NO:60). This resulted in a 200-fold increase in β-Gal activity in E. faecalis, compared to the lacZ RBS (GGAGG(N)₆ATG; SEQ ID NO:61) used above. consequently, the Gram-positive RBS was also incorporated into the forward primers amplifying c1 and bof.

The reporter plasmids were constructed in pAM401, which contains a p15A replicon derived from pACYC184 and a pGB354 replicon derived from the broad-host-range Gram-positive plasmid pIP501 (Wirth et al., J. Bacteriol., 165:831-836 (1986)). Consequently, the plasmid can be used for studies in enteric Gram-negative bacteria, Streptococcus species, Enterococcus species, Streptococcus gordonii, L. lactis, Lactobacillus casei, and Pediococcus species.

To demonstrate the functionality of the promoter system, β-Gal activity was measured in E. coli. β-Gal activity was measured using three C1-regulated promoters driving lacZ at the permissive (31° C.) and non-permissive temperatures (42° C.). In the absence of C1 polypeptide, the activities of all three promoters were high with P102 and P103 producing about 5- to 10-fold more Miller units than P101 (Table 11). This was most likely due to the one nucleotide change from ‘G’ to the consensus ‘T’ within the −10 hexamer in P102 and P103 (FIG. 17). P102 and P103 exhibited similar activities indicating that the ‘TG’ dinucleotide had little effect on promoter strength in E. coli. In the presence of C1 polypeptide and at low temperature, P-Gal activity was significantly reduced indicating that C1 polypeptide can efficiently repress transcription from these promoters. In particular, the basal expression of P102 was much lower than P103, which was probably a reflection on the number of mismatches in the C1 operator sites (one and five mismatches, respectively), and hence the ability to more effectively repress transcription. Interestingly, the basal expression of P102 also was lower than the control vector, which contained a promoterless lacZ gene. This may be explained by the observation that in E. coli, repressor bound operators can prevent the formation of active complexes between RNA polymerase and promoters, and also terminate ongoing transcription (Deuschle et al., PNAS, 83:4134-4137 (1986)). Little difference was observed in the basal levels of expression when C1 polypeptide was expressed from P201 or P202, suggesting that adequate amounts of C1 polypeptide were produced from both constructs to repress transcription effectively. At the non-permissive temperature, β-Gal activity significantly increased from the C1-regulated promoters, although still below fully induced levels (Table 11). Nevertheless, the range of regulation was similar to the bacteriophage P1-derived C1-regulated promoter system described above in E. coli. Thus, controlled expression was achieved in E. coli using Gram-positive transcriptional and translational preferred elements and a synthetic C1-regulated promoter.

TABLE 11
Basal and induced activities from lacZ fusions to C1-regulated
promoters in E. coli DH5α cells.

| | Presence of | Activity (Miller units) | |
|---|---|---|---|
| Construct | C1 repressor | Basal (31° C.) | Induced (42° C.) |
| Control | — | 4.2(0.5) | 6.0(0.7) |
| P101lacZ | — | 1117.5(223.4) | 2197.1(77.9) |
| P102lacZ | — | 15478.9(675.7) | 10899.2(531.1) |
| P102lacZ | P201 | <0.25 | 15.9(1.2) |
| P102lacZ | P202 | <0.25 | 5.7(0.1) |
| P103lacZ | — | 9119.0(272.4) | 9575.1(666.2) |
| P103lacZ | P201 | 2.8(0.1) | 2386.1(504.8) |
| P103lacZ | P202 | 2.0(0.3) | 213.1(11.1) |

Overnight cultures were diluted 1:100 and grown to an OD₆₀₀ of about 0.1 at 31° C. The cultures were then divided equally and incubated at 31° C. or 42° C. for 95 minutes prior to being assayed for β-Gal activity (OD₆₀₀ about 0.6). The control strain carried a plasmid containing a promoterless lacZ gene. Values are averages (± standard deviation) for multiple cultures (n = 3) assayed in triplicate.
<0.25 indicates below the limits of detection for the assay.

Many of the E. coli regulated promoter systems fail to function in Gram-positive species primarily due to (1) more stringent promoter requirements and (2) the requirement that the inducer be actively transported into the cell. Utilizing temperature as the trigger for induction circumvents this limitation. The C1-regulated promoters were analyzed in E. faecium, E. faecalis, and S. aureus (Table 12). In the absence of C1 polypeptide, the activity of P101 was low to undetectable. However, expression from P102 was high indicating that the one nucleotide difference between P101 and P102, in contrast to E. coli, was needed for activity in these species. The addition of the ‘TG’ dinucleotide (P103) further increased the strength of the promoter. In the presence of C1 polypeptide at the permissive temperature, the basal activity of P102 was reduced to the background level displayed by the control strain carrying the promoterless lacZ construct. This indicated that the P102 promoter was completely repressed in the presence of C1 polypeptide, a result similar to the results demonstrated above in Gram-negative bacteria. Tight control is an important feature for regulated promoter systems, since it enables cloning of genes encoding toxic products and the isolation and study of null mutations in essential genes.

TABLE 12
Basal and induced activities from lacZ fusions to C1-regulated
promoters in E. faecium, E. faecalis, and S. aureus.

| Species and | Presence of | Activity (Miller units) | |
|---|---|---|---|
| construct | C1 repressor | Basal (31° C.) | Induced (42° C.) |
| E. faecium | | | |
| Control | — | 1.3(0.1) | 0.6(0.03) |
| P101lacZ | — | 1.7(0.4) | 1.4(0.35) |
| P102lacZ | — | 1769.9(89.6) | 3849.2(131.6) |
| P102lacZ | P201 | 1.8(0.3) | 1.6(0.1) |
| P102lacZ* | P202 | 3.4(0.2) | 640.3(14.5) |
| P103lacZ | — | 2344.6(165.1) | 2564.3(387.7) |
| P103lacZ | P201 | 227.6(10.8) | 699.0(57.2) |
| P103lacZ | P202 | 825.1(16.3) | 1528.1(65.4) |
| E. faecalis | | | |
| Control | — | <0.25 | <0.25 |
| P101lacZ | — | <0.25 | <0.25 |
| P102lacZ | — | 1139.1(23.6) | 3068.3(119.7) |
| P102lacZ | P201 | <0.25 | <0.25 |
| P102lacZ | P202 | <0.25 | 269.0(49.5) |
| P103lacZ | — | 2332.4(54.6) | 4860.7(149.8) |
| P103lacZ* | P201 | 2.7(1.2) | 758.7(366.1) |
| P103lacZ | P202 | 884.0(145.4) | 1120.5(29.1) |
| S. aureus | | | |
| Control | — | <0.25 | <0.25 |
| P101lacZ | — | <0.25 | <0.25 |
| P102lacZ | — | 76.1(7.9) | 183.4(35.5) |
| P102lacZ | P201 | <0.25 | <0.25 |
| P102lacZ | P202 | <0.25 | 4.6(0.74) |
| P103lacZ | — | 129.5(16.8) | 257.8(55.1) |
| P103lacZ* | P201 | <0.25 | 26.4(5.8) |
| P103lacZ | P202 | 54.6(3.8) | 138.4(9.1) |
| Overnight cultures were diluted 1:100 and grown to an OD₆₀₀ of about 0.1 at 31° C. The cultures were then divided equally and incubated at 31° C. or 42° C. for 120 minutes (E. faecium), 95 minutes (E. faecalis), or 75 minutes (S. aureus) prior to being assayed for β-Gal activity (OD₆₀₀ about 0.6). The control strain carried a plasmid containing a | | | |
| # promoterless lacZ gene. Values are averages (±standard deviation) for multiple cultures (n = 3) assayed in triplicate. | | | |

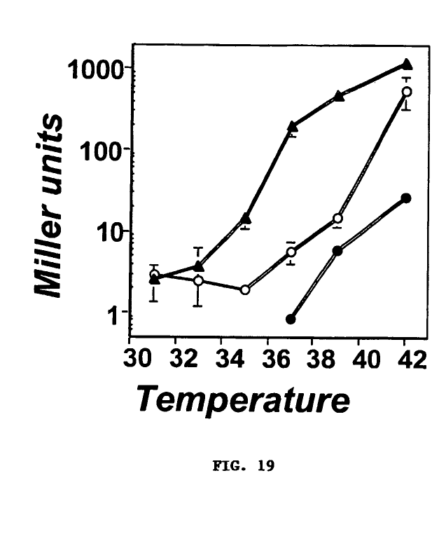

*denotes the reporter constructs used in FIGS. 19-21.

In E. faecalis and S. aureus, the basal level of expression was below the limits of detection when C1 polypeptide was expressed from either P201 or P202. In E. faecium, however, basal activity was slightly higher when P202, as compared to P201, was used to drive C1 polypeptide expression suggesting the ability to repress transcription was dependent on the levels of C1 polypeptide expressed.

In contrast to the low basal expression exhibited by P102, P103 generally resulted in higher basal expression and was more dependent on the promoter driving C1 polypeptide expression and presumably, concentration of repressor present. The higher basal expression may be a reflection of the increased number of mismatches in the C1-operator sites as compared to P102 (five compared to one) leading to less efficient binding of the C1 polypeptide repressor. Moreover, since this promoter was generally stronger, it may also reflect the increased ability of RNA polymerase to compete with the repressor for binding to the unoccupied promoters. Nevertheless, low basal expression was still observed in S. aureus and E. faecalis when C1 polypeptide was expressed from the P201 promoter.

Under inducing conditions from the P102 promoter, a striking difference in the levels of induced expression was achieved depending on whether P201 or P202 was used to drive C1 polypeptide expression (Table 12). Induction was not observed when the P201 promoter was used in combination with the P102 promoter. In contrast, high induced activity was obtained using P202 to drive C1 polypeptide expression, albeit still below fully derepressed levels. This suggested differences in C1 polypeptide expression correlated with the ability to achieve derepression. P201 might be expected to be more active than P202 resulting in higher levels of C1 polypeptide expressed since it contains more conserved nucleotides. However, low levels of basal activity and elevated induced expression were obtained in S. aureus and E. faecalis using P103 promoter irrespective of the promoter used to drive C1 polypeptide expression. This suggests that induced expression depends on both the interaction between the repressor and operator site as well as the amount of repressor present. C1 polypeptide has also been shown to be more thermally stable once tightly bound to DNA as compared to its unbound form which can only be dissociated by further temperature increases (Heinrich et al., Nucleic Acids Res., 17:7681-7692 (1989).

It should be noted that induced expression was achieved in E. coli with these constructs irrespective of the promoters utilized (Table 11). Nevertheless, these results demonstrated that a temperature sensitive C1-regulated promoter can be effectively repressed to levels comparable to the control vectors yet yield high levels of induced expression. Induction/repression ratios for E. faecium, E faecalis, and S. aureus were about 200-fold, 1000-fold, and 100-fold, respectively. Consequently, these results represent the first heterologous regulated promoter system to be described for E. faecium and provides a range of regulation in E. faecalis, which is similar to the promoter systems described for E. coli (Guzman et al., J. Bacteriol., 177:4121-4130 (1995)). The level of regulation achieved for S. aureus is comparable to, if not better than, previously described promoter systems (Ji et al., J. Bacteriol., 181:6585-6590 (1999) and Zhang et al., Gene, 225:297-305 (2000)). In addition, since different combination of promoters were evaluated, constructs can be selected depending on whether tight basal or highly induced expression is preferred.

To analyze the regulation of lacZ expression at the transcriptional level, slot blot analysis was performed (Leonhardt and Alonso, J. Gen. Microbiol., 134:605-609 (1988)). Since promoters were located in both orientations in the plasmid, slot blot analysis was performed using a lacZ complementary oligonucleotide as a probe. RNA was prepared from cultures carrying (1) promoterless lacZ control constructs, (2) reporter constructs lacking c1 repressor, and (3) reporter constructs under repressed and derepressed conditions. The blots were also hybridized with a complementary oligonucleotide homologous to a conserved region of 16s rRNA to verify equal loading of the RNA. LacZ expression from the promoterless lacZ control constructs and the constructs lacking c1 were low and high as expected. Furthermore, the level of lacZ transcripts produced from the control vectors and reporter constructs under repressed conditions were similar indicating C1 polypeptide can efficiently repress transcription. In contrast, at elevated temperatures, lacZ expression from the reporter constructs was significantly increased. The results are therefore in agreement with enzymatic assays and confirmed that the regulation of lacZ expression occurred primarily at the level of transcription.

The ability to obtain different levels of expression by partial induction of the promoter is an important feature of a controlled expression system. Therefore, to assess the ability to modulate expression driven by the temperature sensitive C1-regulated promoter in E. faecium, E. faecalis, and S. aureus, P-Gal activity was measured at different temperatures. The results indicated that by varying temperature, it was possible to modulate expression (FIG. 19). However, the degree to which the promoter could be modulated varied with each host. For example, in E. faecalis, there was a steady increase in β-Gal activity as the temperature increased. In contrast, the level of β-Gal expressed in E. faecium remained relatively unchanged until 39° C. For all three species, maximal induction was achieved at the highest temperature tested (42° C.), which is in agreement with results indicating C1 instability at 42° C. and above. Since Enterococci can tolerate temperatures of 45° C. (Huycke et al., Emerg. Infect. Dis., 4:239-249 (1998)), higher induced activities may be observed by a further temperature increase.

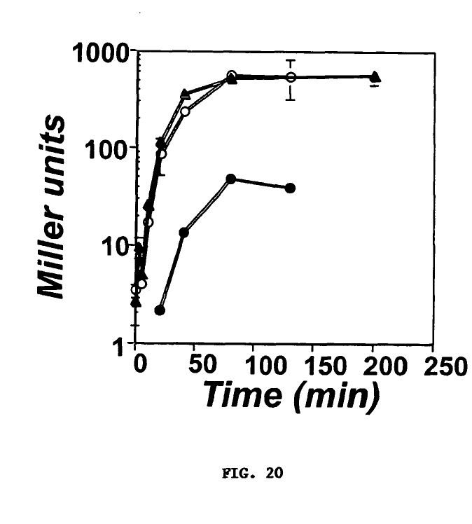

To examine the kinetics of induction, the cultures were grown at low temperature and then induced at the elevated temperatures. At the indicated times, cultures were harvested and β-Gal activity was measured. The kinetics of induction for E. faecium, E. faecalis, and S. aureus were similar and indicated that the temperature sensitive C1-regulated promoter has a fast rate of induction. In addition, the results indicated that incubation under inducing conditions need only be maintained for 80 minutes to achieve maximal induction (FIG. 20).

In summary, the Gram-negative bacteriophage P1 temperature sensitive C1 repressor polypeptide can be used to control gene expression in clinically relevant Gram-positive bacteria. For all three species tested, the promoters were shown to be tightly repressed, an essential characteristic of a promoter system. In E. faecalis, the level of regulation was 1000-fold, bringing a level of efficiency comparable to promoter systems currently used in Gram-negative bacteria. Furthermore, significant regulation was obtained in E. faecium, a species in which no heterologous regulated promoter systems have been described.

The C1-regulated promoters and promoters driving C1 expression were designed based upon conserved Gram-positive promoter elements and thus should be active in a wide variety of bacteria. The vectors also were constructed in a broad-host-range vector capable of replication in Gram-positive species as well as enteric Gram-negative species. Tight basal expression and controlled induction using the same reporter plasmid was demonstrated in both E. coli and Gram-positive species, a feature that may have many applications. Furthermore, as temperature is the inducer, the promoter system is not dependent on exogenously supplied inducers. For these reasons, the temperature sensitive regulated promoter system can be used for genetic studies in both pathogenic Gram-negative and Gram-positive species.

### Example 11

### Construction of Bacteriophage P1 Mutants that are Able to Package Transfer Plasmids But are Unable to Package P1 DNA

A P1 lysogen lacking an initiation site for packaging unable to package its own DNA but capable of producing phage particles containing transfer plasmid DNA is constructed. The transfer plasmid is packaged preferentially within the pool of viral and bacterial DNA since it is the only DNA to contain a pac site.

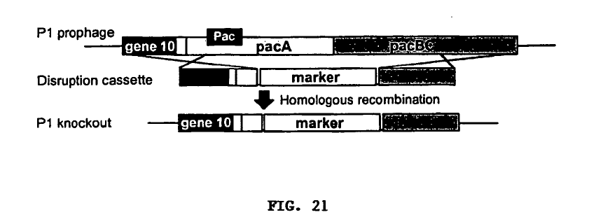

A P1 lysogen in which the phage pac site has been deleted is produced. Gene replacement is performed using a technique that relies on homologous recombination between the wild-type P1 prophage and an in vitro-altered DNA fragment (FIG. 21). The minimal P1 pac site is 161 base pairs and lies within the coding sequence of the pacA gene. Pac A is part of a cotranscribed cluster of three genes that encode the subunits of the pacase enzyme. PacA is located upstream of pacB, and pacC is encoded within the C-terminal end of the pacB gene. Disruption of the pac site will automatically disrupt pacAB and can affect downstream expression of pacC (FIG. 21). To compensate for these possible polar effects, one can complement in trans the P1 pac site mutants with pacABC from a multicopy plasmid.

The disruption vector contains a nutritional or antibiotic marker, such as the TRP1 gene from Saccharomyces cerevisiae, flanked by sequences homologous to the P1 prophage. At least 240 base pairs of homology is used to achieve the second crossover event. P1 DNA segments are cloned from P1 phage lysates by PCR. The disruption cassette is PCR amplified using phosphorothioate-linked P1-specific primers. Phosphorothioate groups are incorporated into the first, second, and third positions from the 5′ end of the linear DNA fragment and render the ends more nuclease-resistant. Since the linear disruption cassette is protected from exonucleases, it is not necessary to perform transformations in a recBC sbcB or recD deficient strain. The only requirement for the host strain is that it is recombination proficient.

To obtain the P1 pac site knockout, P1 lysogens (recA+) are electroporated with the phosphorothioate protected disruption cassette. A double crossover event between the in vitro-altered sequence and the P1 prophage results in deletion of the pac site and acquisition of a nutritional or antibiotic marker. P1 lysogens carrying a pac site deletion are screened initially for the ability of the antibiotic marker to confer antibiotic resistance or complementation of an E. coli auxotrophic strain. Replacement of the pac site is verified by PCR and Southern blot analysis. Gene replacements and deletions are generated in E. coli using standard methods.

The desired mutants can represent a small fraction of the transformants, and a phenotypic screen for the mutant may be needed. In this situation, P1Cm c1ts100 transformants are plated at 32° C. Replica plated colonies are induced into vegetative growth and transferred onto a lawn of Tet-resistant target cells. Lysogens capable of packaging their DNA would infect the target cell and produce a Tet-resistant Cm-resistant colony. P1 disruptants are detected by their functional inability to form such a colony.

Thermoinducible P1 Cm lysogens deleted for pac are tested for their inability to package their own DNA. The chloramphenicol marker carried by the P1 prophage is used as a marker for transfer of P1 DNA. P1 lysates are prepared and assayed for lysogen formation by transfer of the chloramphenicol marker to recipient cells and for the ability to form plaques. Electron microscopy is used to determine the phenotypes of P1 mutants and test for the absence of any defects in particle morphogenesis.

P1 pac deletion mutants can be free of defects in late protein synthesis. Heat induction of mutant lysogens results in cell lysis at the normal lysis time for P1. Phage particles produced from P1 pac deletion mutants should be unable to transfer the chloramphenicol marker associated with the P1 genome or form plaques. Result demonstrated that a P1 pac deletion mutant was incapable of forming chloramphenicol resistant lysogens. Electron microscopic analysis is performed to confirm that morphologically intact phage particles lack DNA.

In order to enable the P1 pac site mutants to package the transfer plasmid, the pacABC genes are expressed in trans from a multicopy plasmid. P1 pacABC nucleic acid is expressed from an early P1 promoter Pr94. Two phage encoded polypeptides, the C1 repressor and Bof modulator, are used to regulate transcription from the Pr94 promoter. The C1 repressor polypeptide can have the c1ts100 mutation such that it is temperature sensitive. The complementing plasmid is transformed into the P1Cm c1ts100 pac deleted lysogens harboring the transfer plasmid, and lysis is induced by heat shock treatment. This switch can lead to derepression of Pr94, expression of pacABC in trans, and cleavage of the pac site on the transfer plasmid. The transfer plasmid is packaged into the empty phage heads, and particle formation is completed. P1 viral DNA deleted for pac lacks a recognition site for the pacase enzymes and is therefore not packaged.

Vector construction is completed sequentially to ensure complete repression of the Pr94 promoter. Induction of the P1 pac deletion mutants harboring the trans complement pacABC plasmid and transfer plasmid can result in normal cell lysis and production of morphologically intact phage particles. Infection of a target cell with phage containing transfer plasmid DNA can produce colonies which contain the transfer plasmid but lack P1 viral DNA. If P1 pac mutants package their own DNA at a low frequency, low-frequency P1 transducing mutants can be used.

Simultaneous expression of PacABC polypeptides can cause the plasmid from which they are being expressed to be cleaved, thereby preventing further expression of the pacABC genes. Self cleavage is prevented or engineered to be inefficient by modifying the DNA sequence of the pac site without altering the PacA encoding sequence. The pac site contains seven hexanucleotide elements that are necessary for efficient cleavage by the P1 pacase enyzme. Removal of just one of those elements from either side of the minimal site reduces cleavage by about 10-fold. Moreover, removal of all three elements from the right side of pac reduces cleavage 1000-fold.

### Example 12

### LADS™

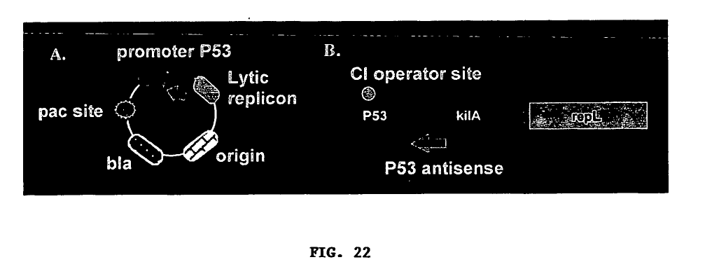

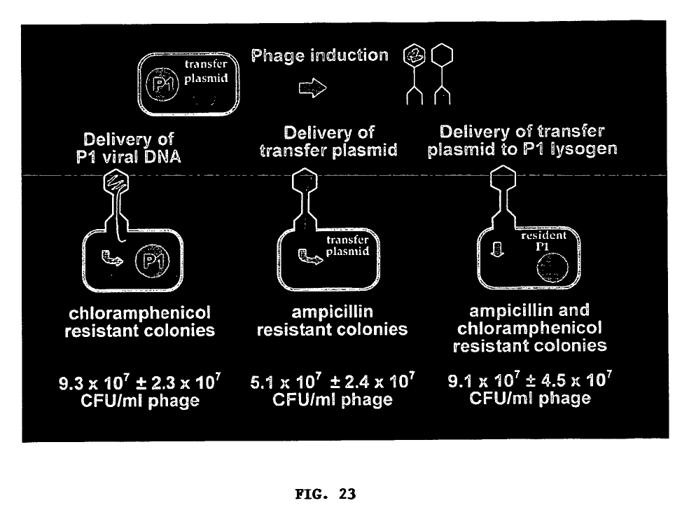

A bacteriophage P1 system (FIG. 22) was used to package and deliver transfer plasmids to E. coli and P. aeruginosa. For example, two transfer plasmids capable of being efficiently packaged in P1 virions for delivery to pathogenic Gram-negative bacteria were developed. The delivery system was not under the constraints of superinfection exclusion (FIG. 23). The phage-based system was not blocked by resident phage such as P1 and lambda, or by compatible plasmids. This is relevant because analyses of environmental samples suggests that up to 40 percent of P. aeruginosa strains in the natural ecosystems (lake water, sediment, soil, and sewage) contain DNA sequences homologous to phage genomes. In addition, the feasibility of using this bacteriophage based system to transfer genetic information in vivo by delivery of a transfer plasmid expressing an antibiotic marker to E. coli and P. aeruginosa in a mouse peritonitis model of infection was demonstrated. Plasmid transfer was confirmed by restriction analysis and sequencing of the plasmid DNA re-isolated from bacteria recovered from the intraperitoneal space.

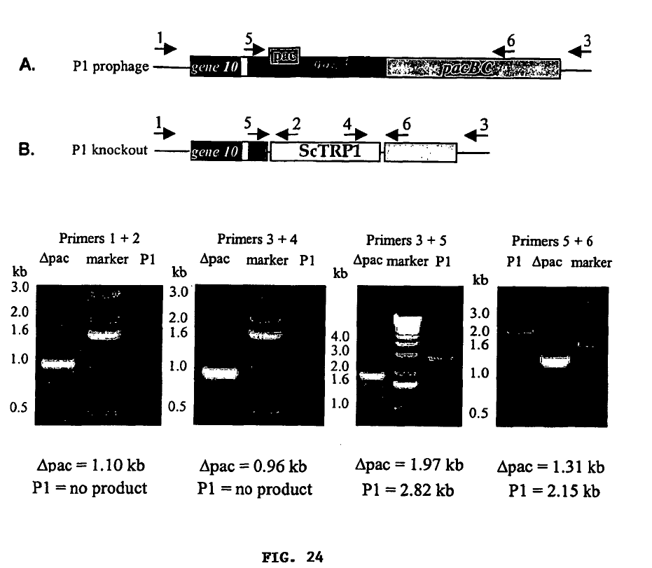

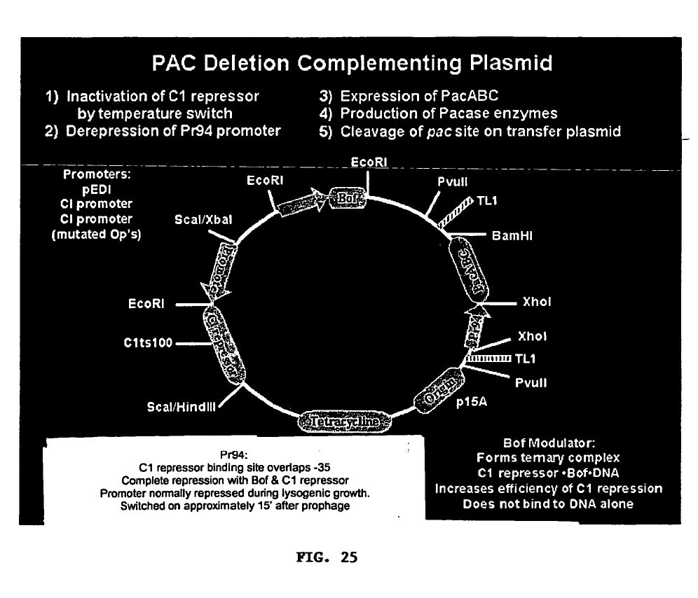

Bacteriophage P1 knockouts able to package transfer plasmid DNA but unable to incorporate P1 DNA were developed. One limitation of using unmodified phage as a delivery vehicle is the potential risk of lysogenic conversion. The P1 knockouts provided herein prevent horizontal transfer of undesirable products to non-pathogenic resident microflora. Phage-mediated transfer of undesirable products to non-pathogenic indigenous microflora is avoided by the inability of the phage to transfer its DNA to the host. The P1 packaging system only packages the transfer plasmid that carries genetic elements for expression of, for example, bactericidal polypetides, into P1 virions for delivery to target pathogenic bacterium. Generation of apac site knockout was constructed and tested (FIGS. 21 and 24). Specifically, the engineered phage were unable to transfer the chloramphenicol marker associated with its genome, suggesting that phage particles produced from the pac mutants lack phage DNA. As a consequence of the pac site lying within the pacABC operon, the modified phage were complemented in trans with the pacase enzyme via a pacABC complementing plasmid (FIG. 25).

Complementation with the pacase enzymes allowed the P1 pac mutants to package the transfer plasmid. A portion of the phage particles produced from the pac mutants, however, contained P1 viral DNA. Analysis of the chloramphenicol resistant transductants indicated that the majority were unable to produce a second round of multiplication, suggesting that they were defective lysogens. The pac mutants appeared to have acquired a pac site, by recombination with the complementing plasmid, thereby enabling the mutants to package and deliver its own viral DNA.

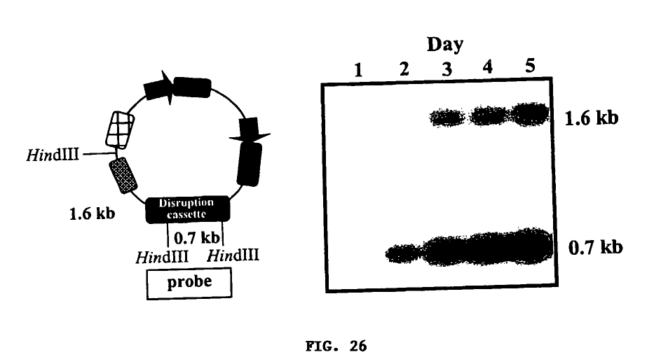

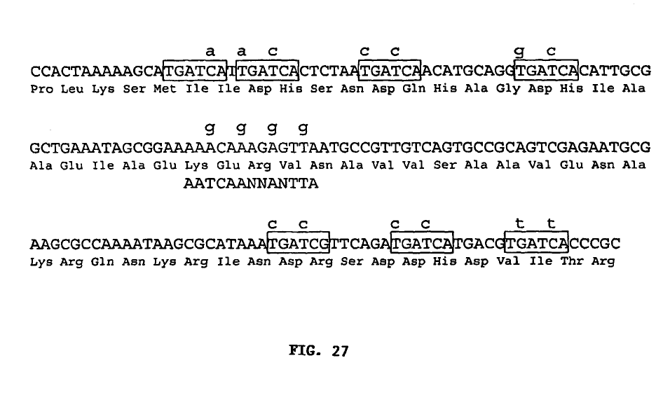

Southern blot analysis verified that the pacABC genes on the complementing plasmid had been replaced with the ScTRP1 disrupted copy (FIG. 26). Silent mutations were introduced into the complementing plasmid pac site so that if any recombination occurs, a defective pac site is introduced into the P1 pac knockout (FIG. 27).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated nucleic acid comprising a C1-regulated promoter sequence operably linked to a nucleic acid sequence, and a promoter sequence operably linked to a second nucleic acid sequence, wherein said C1-regulated promoter sequence and said nucleic acid sequence are heterologous, and wherein said promoter sequence and said second nucleic acid sequence are heterologous.

2. The isolated nucleic acid of claim 1, wherein a cell containing said isolated nucleic acid expresses at least about 10 times less of said nucleic acid sequence when said cell expresses a C1 polypeptide than when said cell does not express said C1 polypeptide.

3. The isolated nucleic acid of claim 2, wherein said cell is a gram-negative bacterial cell.

4. The isolated nucleic acid of claim 3, wherein said gram-negative bacterial cell is a member of a family selected from the group consisting of Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, and Chromobacterium.

5. The isolated nucleic acid of claim 2, wherein said cell is a gram-positive bacterial cell.

6. The isolated nucleic acid of claim 5, wherein said gram-positive bacterial cell is a member of a family or genus selected from the group consisting of Bacillaceae, Sporolactobacillus, Sporocarcina, Filibacter, Caryophanum, Peptococcus, Peptostreptococcus, Ruminococcus, Sarcina, Coprococcus, Mycobacteriaceae, Actinomyces, Bifidobacerium, Eubacterium, Propionibacerium, Staphylococci, Streptococci, Lactococcus, Lactobacillus, Corynebacterium, Erysipelothrix, and Listeria.

7. The isolated nucleic acid of claim 1, wherein a cell containing said isolated nucleic acid expresses at least about 100 times less of said nucleic acid sequence when said cell expresses a C1 polypeptide than when said cell does not express said C1 polypeptide.

8. The isolated nucleic acid of claim 1, wherein a cell containing said isolated nucleic acid expresses at least about 1000 times less of said nucleic acid sequence when said cell expresses a C1 polypeptide than when said cell does not express said C1 polypeptide.

9. (canceled)

10. (canceled)

11. The isolated nucleic acid of claim 1, wherein said C1-regulated promoter sequence comprises a sequence at least about 85 percent identical to the sequence set forth in SEQ ID NO:2, SEQ ID NO:3, SEQ ID NO:4, SEQ ID NO:5, SEQ ID NO:18, or SEQ ID NO:19.

12. The isolated nucleic acid of claim 1, wherein said C1-regulated promoter sequence comprises a sequence at least about 95 percent identical to the sequence set forth in SEQ ID NO:2, SEQ ID NO:3, SEQ ID NO:4, SEQ ID NO:5, SEQ ID NO:18, or SEQ ID NO:19.

13. The isolated nucleic acid of claim 1, wherein said nucleic acid sequence encodes a polypeptide.

14. The isolated nucleic acid of claim 13, wherein said polypeptide is a bacterial polypeptide.

15. The isolated nucleic acid of claim 13, wherein expression of said polypeptide in a bacterial cell kills said bacterial cell.

16. The isolated nucleic acid of claim 13, wherein said polypeptide is a Doc polypeptide.

17. (canceled)

18. The isolated nucleic acid of claim 1, wherein said promoter sequence is an inducible promoter sequence.

19. The isolated nucleic acid of claim 18, wherein said inducible promoter sequence is an AraBAD promoter sequence, a T7 promoter sequence, a LacR/O promoter sequence, a TetR/O promoter sequence, or an AraC/IL-12 promoter sequence.

20. The isolated nucleic acid of claim 18, wherein said inducible promoter sequence is a LacI-regulated promoter sequence.

21. (canceled)

22. (canceled)

23. The isolated nucleic acid of claim 20, wherein said LacI-regulated promoter sequence comprises a sequence at least about 85 percent identical to the E. coli LacI promoter.

24. The isolated nucleic acid of claim 20, wherein said LacI-regulated promoter sequence comprises a sequence at least about 95 percent identical to the E. coli LacI promoter.

25. The isolated nucleic acid of claim 1, wherein said second nucleic acid sequence encodes a polypeptide.

26. The isolated nucleic acid of claim 25, wherein said polypeptide is a C1 polypeptide.

27. The isolated nucleic acid of claim 25, wherein said polypeptide is a temperature sensitive C1 polypeptide.

28. The isolated nucleic acid of claim 27, wherein binding of said temperature sensitive C1 polypeptide to said C1-regulated promoter sequence is inhibited when the temperature is greater than 37° C. as compared to the binding that occurs at 31° C.

29. The isolated nucleic acid of claim 27, wherein binding of said temperature sensitive C1 polypeptide to said C1-regulated promoter sequence is inhibited when the temperature is greater than 40° C. as compared to the binding that occurs at 31° C.

30. The isolated nucleic acid of claim 27, wherein said promoter sequence is a LacI-regulated promoter sequence.

31. The isolated nucleic acid of claim 30, wherein a cell containing said isolated nucleic acid expresses at least about 10 times more of said nucleic acid sequence when said cell is exposed to 42° C. and 0 mM IPTG as compared to when said cell is exposed to 31° C. and 10 mM IPTG.

32. The isolated nucleic acid of claim 31, wherein said cell is a gram-negative bacterial cell.

33. The isolated nucleic acid of claim 32, wherein said gram-negative bacterial cell is a member of a family selected from the group consisting of Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, and Chromobacterium.

34. The isolated nucleic acid of claim 31, wherein said cell is a gram-positive bacterial cell.

35. The isolated nucleic acid of claim 34, wherein said gram-positive bacterial cell is a member of a family or genus selected from the group consisting of Bacillaceae, Sporolactobacillus, Sporocarcina, Filibacter, Caryophanum, Peptococcus, Peptostreptococcus, Ruminococcus, Sarcina, Coprococcus, Mycobacteriaceae, Actinomyces, Bifidobacerium, Eubacterium, Propionibacerium, Staphylococci, Streptococci, Lactococcus, Lactobacillus, Corynebacterium, Erysipelothrix, and Listeria.

36. The isolated nucleic acid of claim 30, wherein a cell containing said isolated nucleic acid expresses at least about 100 times more of said nucleic acid sequence when said cell is exposed to 42° C. and 0 mM IPTG as compared to when said cell is exposed to 31° C. and 10 mM IPTG.

37. The isolated nucleic acid of claim 30, wherein a cell containing said isolated nucleic acid expresses at least about 1000 times more of said nucleic acid sequence when said cell is exposed to 42° C. and 0 mM IPTG as compared to when said cell is exposed to 31° C. and 10 mM IPTG.

38. The isolated nucleic acid of claim 30, wherein said isolated nucleic acid comprises a sequence encoding a LacI polypeptide.

39. The isolated nucleic acid of claim 38, wherein said LacI polypeptide is a temperature sensitive LacI polypeptide.

40. The isolated nucleic acid of claim 39, wherein binding of said temperature sensitive LacI polypeptide to said LacI-regulated promoter sequence is inhibited when the temperature is greater than 37° C. as compared to the binding that occurs at 31° C.

41. The isolated nucleic acid of claim 39, wherein binding of said temperature sensitive LacI polypeptide to said LacI-regulated promoter sequence is inhibited when the temperature is greater than 40° C. as compared to the binding that occurs at 31° C.

42. The isolated nucleic acid of claim 39, wherein said nucleic acid sequence encodes a second polypeptide.

43. The isolated nucleic acid of claim 42, wherein a cell containing said isolated nucleic acid expresses at least about 10 times more of said second polypeptide when said cell is exposed to 42° C. as compared to when said cell is exposed to 31° C.

44. The isolated nucleic acid of claim 42, wherein a cell containing said isolated nucleic acid expresses at least about 100 times more of said second polypeptide when said cell is exposed to 42° C. as compared to when said cell is exposed to 31° C.

45. The isolated nucleic acid of claim 42, wherein a cell containing said isolated nucleic acid expresses at least about 1000 times more of said second polypeptide when said cell is exposed to 42° C. as compared to when said cell is exposed to 31° C.

46-85. (canceled)
